(19) United States
(12) Patent Application Publication
(10) Pub. No.: US 2026/0128122 A1
(43) Pub. Date: May 7, 2026

(54) ACCURATE ALLELE-SPECIFIC SOMATIC COPY NUMBER CALLING FROM PICOGRAM QUANTITIES OF DNA

(71) Applicant: Oxford University Innovation Limited, Oxford (GB)

(72) Inventors: Joel NULSEN, Oxford (Oxfordshire) (GB); Ahmed AHMED, Oxford (Oxfordshire) (GB)

(21) Appl. No.: 19/117,678

(22) PCT Filed: Sep. 29, 2023
(86) PCT No.: PCT/GB2023/052521

§ 371 Date: Apr. 2, 2025

(30) Foreign Application Priority Data
Oct. 3, 2022 (GB) 2214524.7
Mar. 16, 2023 (GB) 2303893.8

(51) Int. Cl. G16B 20/20; C12N 15/10; G16B 35/10; G16B 40/20

(52) U.S. Cl. CPC G16B 20/20; C12N 15/1065; G16B 35/10; G16B 40/20

(57) ABSTRACT

The invention relates to a method of determining somatic allele-specific copy number alterations (CNAs) in the genomes of cells in a test-sample from a subject, the method comprising: i) providing an indexed-DNA library of DNA fragments resulting from whole-genome amplification of genomic DNA from cells of the test-sample, ii) providing whole genome sequencing data of reference non-cancer cells from a reference-sample from the subject; and iii) determining somatic allele-specific copy number alterations in the genome(s) of the cells of the test-sample; and associated methods and uses in cancer therapy.

This invention relates to a method of determining somatic allele-specific copy number alterations (CNAs) in the genomes of cells in a test-sample from a subject.

Cancer is a genomic disease. The driving force behind each cancer is a repertoire of genomic changes known as somatic alterations {Bailey, 2018 #6: Consortium, 2020 #7}. Understanding how these alterations drive cancer is one of the central aims of cancer genomics {Vogelstein, 2013 #13}, and efforts in this field have brought about clinical benefits including improved patient stratification, new prognostic biomarkers and an arsenal of new therapies {Berger, 2018 #30: Malone, 2020 #29}.

Copy number alterations (CNAs) are an important class of somatic alterations in cancer in which large segments of the genome are either amplified or deleted. They have been found to drive the cancer phenotype {Sondka, 2018 #9: Wang, 2020 #10}, play a prominent role in cancer evolution {Gerstung, 2020 #8}, and hold substantial prognostic value {Hieronymus, 2018 #12: Smith, 2018 #11}. They are particularly important in genomically unstable cancer types such as ovarian {Penner-Goeke, 2017 #31}, oesophageal {Paulson, 2009 #32} and gastric cancers {Maleki, 2017 #33}. Due to this importance, researchers have developed numerous algorithms to call somatic CNAs from sequencing data, including ASCAT {Van Loo, 2010 #1}, ABSOLUTE {Carter, 2012 #26}, Control-FREEC {Boeva, 2012 #27}, OncoSNP-SEQ {Yau, 2013 #28}, and TITAN {Ha, 2014 #2}, among many others. In addition to determining the total number of copies at each locus in the genome, many of these algorithms also determine the relative numbers of the two parental alleles. Such methods are said to call allele-specific CNAs. This added allele-specific information allows researchers to identify subtle CNAs such as copy number neutral loss of heterozygosity (LOH) {Van Loo, 2010 #1}, which can have important clinical implications in cancer {Ryland, 2015 #23}.

While early efforts tended to analyse bulk tumour samples, recent research has become more directed towards microscopic samples. For example, studies have investigated the therapeutic opportunities associated with circulating tumour cells {Lin, 2021 #39}, minimal residual disease {Artibani, 2021 #42; Luskin, 2018 #40} and cancer sub-populations such as tumour initiating cells {Qureshi-Baig, 2017 #41}. One approach for characterising the genomes of such samples is single cell sequencing. Several CNA-calling algorithms have been developed that combine shallow whole genome sequencing (WGS) data across hundreds of cells to call CNAs at single-cell resolution. Established algorithms for calling total copy number include Ginkgo {Garvin, 2015 #14} and AneuFinder {Bakker, 2016 #3}. More recently, researchers developed allele-specific single cell algorithms, including CHISEL {Zaccaria, 2021 #44} and Alleloscope {Wu, 2021 #43}. However, while they may provide accurate CNA calls, single cell methods are still limited in their accuracy to genotype single nucleotide variants (SNVs) {Dong, 2017 #45}, which are complementary to CNAs and can be highly consequential for therapeutics {Morotti, 2021 #46}. An ideal platform for sequencing microscopic tumour samples would generate both accurate SNVs and CNAs for a complete genomic characterisation. Moreover, many single cell methods rely on aggregating data across hundreds or even thousands of cells, and are therefore unsuitable when total sample size is limited to tens of cells.

To investigate the genomes of tumour samples comprising tens of cells (e.g. obtained by laser capture microdissection), we recently developed a linked-read sequencing platform called DigiPico, which is described in International Patent Application Publication No: WO2021116677A1, and which is incorporated herein by reference. DigiPico allows accurate identification of clonal SNVs and short insertions and deletions (indels) from picogram quantities of DNA {KaramiNejadRanjbar, 2020 #4}. In the DigiPico protocol, we first distribute large fragments of genomic DNA, on the order of 100 kb in length, across one or more 384-well plates. Within each well separately, we then amplify, fragment and barcode the material. Performing the amplification step independently in each well allows us to computationally identify and remove artefactual mutations arising from oxidation and spontaneous deamination. Intuitively, real mutations have support from a larger proportion of wells than artefactual mutations, which tend to appear in only a single well due to the random nature of the DNA damage. We developed a machine learning algorithm, MutLX, to exploit this effect to reliably identify the real mutations in a DigiPico sample {KaramiNejadRanjbar, 2020 #4}.

Given the biological and prognostic importance of somatic CNAs, we sought to develop a method to accurately call somatic allele-specific CNAs from DigiPico sequencing data. We noted that existing CNA calling methods for linked-reads only produced total copy number, and were not allele-specific {Marks, 2019 #47}.

An aim of the invention is to improve methods of determining somatic allele-specific copy number alterations in the genome of cells.

According to a first aspect of the present invention, there is a method of determining somatic allele-specific copy number alterations (CNAs) in the genomes of cells in a test-sample from a subject, the method comprising:
i) providing an indexed-DNA library of DNA fragments resulting from whole-genome amplification of genomic DNA from cells of the test-sample, wherein fragments of DNA in the indexed-DNA library are distributed among, and are indexed to, individual wells, and the fragments are at least 40 kb in length;
ii) providing whole genome sequencing data of reference non-cancer cells from a reference-sample from the subject; and
iii) determining somatic allele-specific copy number alterations in the genome(s) of the cells of the test-sample by:
   a) determining a read depth ratio (RDR), which is the ratio of read counts of an allele in the test-sample relative to the reference-sample, or a log ratio (Log R) thereof, thereby providing the total copy number of the allele, wherein the RDR or Log R value is derived from counts of the allele in reconstructed fragments of DNA that have been reconstructed in silico, and wherein the counts are within a bin of between 100 kb and 5 Mb, or between 80 kb and 120 kb, on the genome; and
   b) determining a B-allele frequency (BAF) value, which is the allelic ratio of parental alleles determined by the ratio of the wells supporting a reference allele versus a non-reference allele and averaged over phased groups of heterozygous single nucleotide polymorphisms (SNPs); and
   c) using the determined RDR or log R, and the BAF value to call allele-specific somatic CNAs via an allele specific CNA calling algorithm, such as a mean-squared error (MSE)-minimisation algorithm.

The method of the invention is herein termed “PicoCNV”. PicoCNV advantageously provides the ability to call allele-specific copy number alterations from picogram quantities of DNA, corresponding to samples of just tens of cells. PicoCNV has been extensively benchmarked and employed to inform the clinical management of a patient with minimal residual disease. Combined with other recently-developed methods, PicoCNV allows for accurate and comprehensive genomic characterisations of cancers in settings where only microscopic samples are available. It is recognised that in DigiPico, short reads are derived from long DNA fragments, distributed into multiple wells. Advantageously, by counting the wells/long DNA fragments instead of short reads, we remove the noise generated by DNA amplification. Averaging BAF values over phased groups of SNPs also reduces noise by aggregating data from multiple individually noisy sources.

### The Allele-Specific CNA-Calling Algorithm

The skilled person will be familiar with allele-specific CNA-calling algorithms that are available for use with the present invention. The allele-specific CNA-calling algorithm may be selected from ASCAT, ABSOLUTE, Control-FREEC, OncoSNP-SEQ, and TITAN. In one embodiment, the allele-specific CNA-calling algorithm is a mean-squared error (MSE)-minimisation algorithm

In another embodiment, the allele-specific CNA-calling algorithm comprises or consists of the TITAN algorithm. The TITAN algorithm is described by Ha, G., et al. (2014. TITAN: Inference of copy number architectures in clonal cell populations from tumour whole genome sequence data. Genome Research, 24: 1881-1893. PMID: 25060187), which is incorporated herein by reference.

In a preferred embodiment, the allele-specific CNA-calling algorithm has a tuneable segment length parameter. In one embodiment, the default segment length parameter of the allele-specific CNA-calling algorithm is increased. In particular, the segment length parameter of the allele-specific CNA-calling algorithm may be larger than the default value of the segment length parameter. The segment length parameter may be increased until copy number segments have a desired length distribution. In one embodiment, the segment length parameter of the allele-specific CNA calling algorithm, such as for TITAN, is set to 10²¹, but it may be set within a range of 10²⁰ to 10²³.

### Determining RDR or LogR

In a preferred embodiment, RDR is determined and used to call allele-specific somatic CNAs. In another embodiment, LogR is determined and used to call allele-specific somatic CNAs. Preferably, the counts are within a bin of between 100 kb and 5 Mb on the genome, for example when using RDR. Alternatively, the counts may be within a bin of between 80 kb and 120 kb on the genome, for example where LogR is used.

### Determining a B-Allele Frequency (BAF)

Step b) (i.e. determining a B-allele frequency (BAF)) comprises averaging the BAF values over phased groups of the heterozygous SNPs. The skilled person will recognise that BAF values can first be derived at each SNP from counts of wells supporting either the reference or alternative allele, for example using a variant calling software such as Platypus to genotype the SNPs in per-well BAM files generated from DigiPico sequencing data. The method of phasing SNPs is to take advantage of the monoallelic well-based nature of DigiPico sequencing data. Specifically, groups of nearby SNPs that share the same reference/alternative status across a set of wells are likely to be in phase with one another. Conversely, a pair of nearby SNPs that have opposite reference/alternative status across a set of wells are likely to be in anti-phase with one another.

### The in Silico Reconstructed Fragments

Two or more sequences of DNA from the same well may be determined to be a read pair from the same in silico reconstructed fragment if there is a gap between them shorter than 100 kb in length. In another embodiment, two or more sequences of DNA from the same well may be determined to be a read pair from the same in silico reconstructed fragment if there is a gap between them shorter than 10 kb in length. In another embodiment, two or more sequences of DNA from the same well may be determined to be a read pair from the same in silico reconstructed fragment if there is a gap between them shorter than 200 kb in length. In another embodiment, two or more sequences of DNA from the same well may be determined to be a read pair from the same in silico reconstructed fragment if there is a gap between them shorter than 20 kb in length.

### Providing an Indexed-DNA Library

Providing an indexed-DNA library of DNA fragments resulting from whole-genome amplification of genomic DNA from cells of the test-sample may comprise carrying out, or obtaining results from, a method of whole genome sequencing of a group of cells for identification of single nucleotide variants in accordance with International Patent Publication No: WO2021116677A1, which is incorporated herein by reference.

In one embodiment, providing an indexed-DNA library resulting from whole-genome amplification of genomic DNA from cells in the test-sample is by carrying out, or obtaining results from, a method of whole genome sequencing of the cells comprising the steps of:
i) providing a multi-well array plate comprising rows and columns of reaction wells;
ii) providing genomic DNA of cells of a test-sample, wherein the genomic DNA is distributed into a plurality of reaction wells on the multi-well array plate, such that there is no more than one single-stranded genomic DNA molecule of any given locus per reaction well,
iii) carrying out whole genome amplification (WGA) of each genomic DNA molecule to provide multiple copies of the genomic DNA molecule in each reaction well;
iv) fragmenting the DNA molecules of each reaction well and ligating a pair of looped adapters at each end or tagmenting using transposase-delivered adapters to form adapted-DNA fragments, wherein the looped adapters or transposase-delivered adapters comprise either a Column Index (Ci) sequence or a Row Index (Ri) sequence, wherein the Ci sequence is common to each looped adapter or transposase-delivered adapter of every reaction well in a column of the multi-well array plate, or wherein each Ri sequence is common to each looped adapter or transposase-delivered adapter of every reaction well in a row of the multi-well array plate;
vi) providing the indexed DNA library by performing indexing PCR on the adapted-DNA fragments, wherein the adapted-DNA fragments are amplified to form indexed PCR products using forward and reverse indexing primers, wherein either a Row Index (Ri) sequence or Column Index (Ci) sequence is introduced by each forward and reverse indexing primers onto each end of the adapted-DNA fragments, such that the resulting indexed PCR products comprise both a pair of flanking Column Index (Ci) sequences that are common to each well of a column and a pair of flanking Row Index (Ri) sequences that are common to each well of a row, and
vii) sequencing the indexed DNA library.

The sequenced DNA library can provide data for determining any single nucleotide variants, determining chromosome structural variations, or determining phasing information in the genome of the cells.

The fragments of the indexed-DNA library of DNA may be between 40 kb and 500 kb in length. In another embodiment, the fragments of the indexed-DNA library of DNA may be between 40 kb and 300 kb in length. In another embodiment, the fragments of the indexed-DNA library of DNA may be between 40 kb and 200 kb in length. In a preferred embodiment, the fragments of the indexed-DNA library of DNA may be between 40 kb and 100 kb in length.

### Cells

The cells in the test-sample may comprise eukaryote cells, such as mammalian cells. In one embodiment, the cells are human. In one embodiment, the cells are at least diploid cells. The cells may be cancerous cells, or pre-cancerous cells. The cells may comprise tumour islets. In one embodiment, the cell may be derived from a tissue biopsy from a subject. The cells may be from a patient having minimal residual disease (MRD).

The cells, such as tumour islets, may be laser-captured micro-dissected cells.

Where DNA from a plurality of cells is being sequenced, the cells may be spatially—related cells. The cells may be co-located in a tumour or a region of a tumour. In another embodiment, the cells may or may not be immediate neighbours.

In one embodiment, the cells in the test-sample may be circulating tumour cells.

The number of cells in a sample, may be between 1 and 100. In another embodiment the number of cells in the sample is between 1 and 80. In one embodiment the number of cells in the sample is between 1 and 50. In one embodiment the number of cells in the sample is between 2 and 50. In one embodiment the number of cells in the sample is between 5 and 50. In another embodiment the number of cells in the sample is between 10 and 50. In another embodiment the number of cells in the sample is between 10 and 30. In another embodiment the number of cells in the sample is between 20 and 30. In another embodiment the number of cells in the sample is between 20 and 40. In another embodiment the number of cells in the sample is between 10 and 40.

Providing Whole Genome Sequencing Data of Reference Non-Cancer Cells from a Reference-Sample from the Subject

The cells from a reference-sample from the subject may be cells from any non-tumour-containing tissue or blood from the subject. In one embodiment, cells from a reference-sample from the subject may be cells from a blood sample. The reference sample cells may not comprise tumour cells.

The sequencing of cells from the reference sample may be dye-sequencing (e.g. Illumina dye-sequencing), nanopore sequencing, or ion torrent sequencing. The skilled person will be familiar with a number of different sequencing technologies/methods that may be used, and the required sequencing adapters therefor

At least about 10⁵ cells may be provided in the reference-sample. Preferably at least about 10⁶ cells may be provided in the reference-sample. The amount of cells in the reference-sample may be sufficient to provide at least 100 mg of DNA.

### Providing Nucleic Acid Molecules and Well Distribution

The nucleic acid may be purified nucleic acid or partial purified nucleic acid. In another embodiment, the nucleic acid may be provided in a cell lysate. The genomic DNA may be provided as purified DNA. In another embodiment, the genomic DNA may be provided from resuspended nuclei, or whole cells, such as laser-captured micro-dissected cells.

The genomic DNA may comprise DNA of a single cell, or a group of cells (cell-group), such as spatially related cells. The genomic DNA may comprise DNA of between about 1 and 30 cells. The genomic DNA may comprise DNA of between about 1 and 100 cells. In another embodiment, the genomic DNA may comprise DNA of between about 1 and 80 cells. In another embodiment, the genomic DNA may comprise DNA of between about 1 and 50 cells. In another embodiment, the genomic DNA may comprise DNA of between about 1 and 40 cells. In another embodiment, the genomic DNA may comprise DNA of between about 10 and 30 cells. In another embodiment, the genomic DNA may comprise DNA of between about 20 and 30 cells. In another embodiment, the genomic DNA may comprise DNA of between about 20 and 40 cells. In another embodiment, the genomic DNA may comprise DNA of between about 10 and 40 cells.

Where the nucleic acid, such as DNA, is double stranded, the nucleic acid may be denatured prior to distribution into the wells. The denaturing may be achieved by heat and/or denaturing buffer. In one embodiment, the nucleic acid, such as genomic DNA, or nuclei or cells containing genomic DNA may be denatured using a denaturing buffer, such as the D2 buffer from Repli-g single cell kit (Qiagen).

The nucleic acid, such as DNA, may be distributed into the wells such that such that there is no more than one single-stranded genomic DNA molecule of any given locus per reaction well. Distribution of the nucleic acid may be facilitated by dilution of the nucleic acid. Therefore, in one embodiment, the nucleic acid solution may be diluted. The skilled person will readily determine the level of dilution and the volume of the solution necessary for achieving no more than one single-stranded genomic DNA molecule of any given locus per reaction well. The skilled person will recognise that the level of dilution necessary may be determined mathematically such that there is a statistically high probability of there being no more than one single-stranded genomic DNA molecule of any given locus per reaction well. For example, Poisson distribution may be used for the calculation when the number of cells is known.

In one embodiment, the DNA content of a single cell may be distributed amongst wells of a single row or column. Therefore, a multi-well array plate may be used to analyse multiple different single cells, such as one per row or column. At least one of the wells may be used for adding the cell and extracting the DNA content. In another embodiment the DNA content of a cell or cell group is distributed amongst wells of both rows and columns of a single multi-well array plate.

The skilled person will recognise that any standard multi-well array plate may be used in the method of the invention. Preferably the multi-well array plate is compatible with any PCR and/or sequencing instruments that may be used. The multi-well array plate may comprise a 384 well plate, such as a 24×16 well plate. In another embodiment, the multi-well array plate may comprise a 1536 well plate. The skilled person will appreciate that a larger number of Ri and/or Ci sequences may be required for indexing larger array plates.

The use of a 384 well multi-well array plate can advantageously provide enough wells for distributing diluted genomic DNA strands of about 20-30 cells, such that wells can be provided with a single DNA molecule.

### Amplification

In an embodiment wherein the nucleic acid is genomic DNA, the amplification of the genomic DNA molecules may comprise Whole Genome Amplification (WGA). The WGA may comprise the step of adding amplification reagent for DNA amplification to the genomic DNA. The amplification reagent for DNA amplification may otherwise be termed “amplification mix”. The skilled person will understand that an amplification mix may comprise all the reagents necessary for amplification of the DNA (i.e. creating multiple copies of the DNA). Such components may comprise reaction buffer, polymerase, and dNTPs. A DNA polymerisation reporter molecule, such as a DNA-binding dye (e.g. Evagreen™) may be provided in the amplification mix, for example to allow monitoring of the amplification reaction using a real-time PCR.). The DNA-binding dye may be constructed of two monomeric DNA-binding dyes linked by a flexible spacer. In the absence of DNA, the dimeric dye can assume a looped conformation that is inactive in DNA binding. When DNA is available, the looped conformation can shift via an equilibrium to a random conformation that is capable of binding to DNA to emit fluorescence.

The amplification reagents may be provided in each well prior to or after the addition of the DNA to the wells.

The skilled person will be able to provide suitable conditions for the amplification reaction to occur, including suitable temperature and incubation times. For example, the plate may be incubated at about 30° C. for at least about 1 hour followed by heat inactivation, for example at about 65° C. for at least 5 minutes.

### Fragmenting and Ligation of Looped Adapters or Transposase-Delivered Adapters

In one embodiment, looped adapters are provided, such that the method comprises a step of fragmenting the DNA molecules of each reaction well and a subsequent ligation reaction to ligate looped adapters to the fragmented DNA. The fragmented DNA may be end repaired prior to the ligation. In an alternative embodiment, transposase-delivered adapters may be provided such that the method comprises fragmenting the DNA molecules by the process of tagmentation. The tagmentation may comprise the provision of a transposase, such as Tn5, carrying oligonucleotides, which are herein termed transposase-delivered adapters. The skilled person will be familiar with the routine technique and reagents for carrying out tagmentation to form the adapted-DNA molecules.

Fragmenting the DNA molecules of each reaction well into multiple dsDNA fragments may comprise direct fragmentation, such as enzymatic or mechanical fragmentation. In one embodiment, the fragmentation of the DNA comprises enzymatic fragmentation.

Fragmenting or tagmentation of the DNA may be provided by the addition of a fragmenting or tagmentation reagent to the DNA in each well. The fragmenting or tagmentation reagent may be concurrently added to each well, for example by the use of a multi-well dispenser, such as an I-DOT (Dispendix, Germany) dispenser or similar. The fragmenting or tagmentation reaction may be timed to provide fragments of the desired size. The skilled person will understand that the timing of the fragmenting or tagmentation reaction can be dependent on the method used, such as the type and level of enzymes provided for the reaction. Therefore, the skilled person may follow standard protocol timings for a given reaction, such as those of a reaction kit.

The fragmenting reagent may comprise restrictions enzymes or nicking enzymes, such as DNase I. Where a nicking enzyme is provided, a single-strand-specific enzyme may be provided that recognizes nicked sites then cleaves the second strand. In one embodiment, a library preparation kit may be used, such as the Lotus DNA library preparation kit (IDT, USA).

Following fragmenting of the DNA to form dsDNA fragments, the dsDNA fragments may be end-repaired and dA-tailed, such that they can be ligated to other DNA molecules, such as the looped adapters. Enzymes for end-repaired and/or dA-tailing may comprise a DNA polymerase, such as T4 DNA polymerase, and a polynucleotide kinase (PNK), such as a T4 polynucleotide kinase. T4 DNA polymerase (in the presence of dNTPs) can fill-in 5′ overhangs and trim 3′ overhangs down to the dsDNA interface to generate the blunt ends. The T4 PNK can then phosphorylate the 5′ terminal nucleotide. A DNA polymerase, such as Taq DNA polymerase, that has terminal transferase activity and leaves a 3′ terminal adenine may be provided for A-tailing.

In one embodiment, fragmentation, end-repair, and dA-tailing of dsDNA are all performed in a single reaction.

In one embodiment, the looped adapters may be introduced onto the fragmented DNA by ligation. Ligation of the looped adapters to the dsDNA fragments may comprise the addition of looped adapters and a ligase, such as T4 DNA ligase.

The looped adapters may comprise an oligonucleotide, such as DNA, having a secondary stem-loop structure. The stem-loop structure may be provided by a single oligonucleotide molecule comprising a pair of complementary sequence regions flanking a loop region, wherein the pair of complementary sequences are arranged to hybridise with each other to form the stem-loop structure of the looped adapter. The looped adapters further encode a Column Index (Ci) sequence or Row Index (Ri) sequence in the stem region.

The Column Index (Ci) sequence or Row Index (Ri) sequence may comprise a pre-determined sequence that is capable of labelling the DNA as from a row or column respectively. The Column Index (Ci) sequence or Row Index (Ri) sequence may be at least three nucleotides in length.

In one embodiment, the ends of the adapted-DNA fragments may be symmetrical. In particular, the looped adapters or transposase-delivered adapters ligated to each end of the dsDNA fragment are identical, such that each dsDNA fragment receives a pair of identical flanking looped adapters or transposase-delivered adapters. The pair of Ci sequences on the same adapted-DNA fragment may be the same. Alternatively, if Ri sequences are provided, the pair of Ri sequences on the same adapted-DNA fragment may be the same.

Advantageously, the provision of two identical Ci or Ri sequences on the adapted-DNA fragments provides a marker to avoid analysis of indexed DNA library sequences that are a result of cross-contamination between different columns, or different rows respectively. In particular, any indexed DNA library sequence that does not have matching Ci sequences at each end can be discarded from the data analysis. Alternatively, if Ri sequences are provided, any indexed DNA library sequence that does not have matching Ri sequences at each end can be discarded from analysis. This can provide a first level of redundancy to eliminate index cross-contamination from the subsequent data, which is a significant problem in the preparation of indexed libraries and their subsequent analysis.

The looped adapters may provide a 3′ or 5′ overhang to aid ligation to the dsDNA fragments. The 3′ or 5′ overhang may be provided when the stem region of looped adapter is hybridised together (i.e. the looped adapter is in a secondary/stem-loop structure). The 3′ or 5′ overhang may correspond to a complementary overhang on the dsDNA fragments that have been end repaired and prepared for ligation. The overhang may comprise a single thymine.

The looped adapter sequence may comprise the sequence of SEQ ID NO: 1, or a functional variant thereof.

Following ligation of the looped adapters, the single-stranded region of looped DNA may be cleaved. The single-stranded region of looped DNA may be enzymatically cleaved, for example by USER (Uracil-Specific Excision Reagent) enzyme, which generates a single nucleotide gap at the location of a uracil present in the loop. Therefore, in one embodiment, the looped adapters may comprise a uracil in the loop region.

### Pooling of a Row of Wells

If Ci sequences are provided in the adapted-DNA fragments, the method may additionally comprise the step of pooling the adapted-DNA fragments of each reaction well in a row prior to the indexing PCR. Alternatively, if Ri sequences are provided in the adapted-DNA fragments, the method may additionally comprise the step of pooling the adapted-DNA fragments of each reaction well in a column prior to the indexing

PCR. The pooled adapted-DNA fragments may then be used for indexing PCR in a single pooled reaction for each row, or each column, depending on which is pooled. In an alternative embodiment, the columns or rows may not be pooled prior to carrying out index PCR.

Advantageously, the pooling of the rows or columns prior to indexing PCR greatly improves the efficiency of the library preparation. For example, for a 16×24 (384) well plate only 16 separate indexing PCR reactions are required if the 16 rows are pooled between the introduction of the looped adapters or transposase-delivered adapters and the indexing PCR steps, instead of 384 indexing PCR reactions if they are not pooled.

### Size Selection and Indexing PCR

Prior to the indexing PCR, the adapted-DNA fragments may be selected for size, for example to also remove the self-ligated adapters from the reaction when applicable. An example of a desired size may be about 300-400 bp in length. The size selection may be provided by isolating or purifying the adapted-DNA fragments of the desired length, for example using a gel, or beads. SPRI beads (Solid Phase Reversible Immobilisation beads) may be used for size selection. SPRI beads can comprise magnetic particles coated with carboxyl groups (in the form of succinic acid) that can bind DNA non-specifically and reversibly.

The indexing PCR may comprise the step of mixing the adapted-DNA fragments with a set of forward and reverse indexing PCR primers and reagents for PCR. The forward and reverse indexing PCR primers may comprise a sequence that is arranged to hybridise to a sequence of the adapted-DNA fragments for priming the polymerisation. The sequences that are arranged to hybridise to sequences of the adapted-DNA fragments for priming the polymerisation may be complementary sequences. The sequences for priming the polymerisation from the forward and reverse indexing PCR primers may be provided by the looped-adapters or transposase-delivered adapters. The sequences for priming the polymerisation from the forward and reverse indexing PCR primers may be flanking the Ci or Ri sequences of the adapted-DNA fragments, such that the Ci or Ri sequences become incorporated in the indexed PCR product.

The complementary sequences for hybridisation, which are provided by the forward and reverse primers, may each be between about 15 and 30 nucleotides in length, such as about 26 nucleotides in length.

In an embodiment wherein the adapted-DNA fragments comprise Ci sequences, the forward and reverse indexing PCR primers may each comprise Ri sequences, for providing a pair of Ri sequences in the indexed PCR product. Where the rows are pooled, the Ri sequences will be added to each adapted-DNA fragment in the pool (from all the wells of a row). Alternatively, where the rows are not pooled, the same Ri sequence may be provided for each well in a row.

In an alternative embodiment wherein the adapted-DNA fragments comprise Ri sequences, the forward and reverse indexing PCR primers may each comprise Ci sequences, for providing a pair of Ci sequences in the indexed PCR product. Where the columns are pooled, the Ci sequences will be added to each adapted-DNA fragment in the pool (from all the wells of a column). Alternatively, where the columns are not pooled, the same Ci sequence may be provided for each well in a column.

The Row Index (Ri) sequences, which may be provided by the forward and reverse primers, may be the same for each adapted-DNA fragment of, or from, a row. Alternatively, the Column Index (Ci) sequences, which may be provided by the forward and reverse primers, may be the same for each adapted-DNA fragment of, or from, a column.

The Row Index (Ri) sequences or Column Index (Ci) sequences provided by the forward and reverse primers may each be at least three nucleotides in length, such as about 8 nucleotides in length.

The resulting ends of the indexed PCR products may be symmetrical. For example, the sequences flanking the original DNA fragment sequence may be symmetrical. The indexed PCR products may comprise the DNA fragments sequence flanked by a pair of identical Ci sequences (i.e. inner flank), and further flanked by a pair of identical Ri sequences (i.e. outer flank). In an alternative embodiment, the indexed PCR products may comprise the DNA fragments sequence flanked by a pair of identical Ri sequences (i.e. inner flank), and further flanked by a pair of identical Ci sequences (i.e. outer flank).

Advantageously, the provision of two identical pairs of Ci or Ri sequences on the indexed DNA fragments in addition to the previously provided Ri or Ci sequences (provided by the looped adapters or transposase-delivered adapters) respectively, provides a further marker to avoid analysis of indexed DNA library sequences that are a result of cross-contamination between different columns, or different rows respectively. In particular, any indexed DNA library sequence that does not have matching Ci sequences at each end can be discarded from the data analysis. Alternatively, if Ri sequences are provided, any indexed DNA library sequence that does not have matching Ri sequences at each end can be discarded from analysis. Providing both pairs of matching Ci and Ri sequences on an indexed DNA fragment can provide a first and second level of redundancy to eliminate index cross-contamination from the subsequent data, which is a significant problem in the preparation of indexed libraries and their subsequent analysis.

The forward and reverse indexing PCR primers may further comprise sequencing adapter sequences, such that sequencing adapters are incorporated into the indexed PCR product. The sequencing adapter sequences on the primers may be 5′.

The sequencing adapters may be terminal on the indexed PCR product. Where sequencing adapter sequences are provided, the resulting ends of the indexed PCR products may not be symmetrical. For example, one end of the indexed PCR product may be adapted with a sequencing adapter that is different to the sequencing adapter of the other end. The skilled person will be aware of sequencing adapters that may be required for a given sequencing technology. For example, in the case of dye sequencing (e.g. Illumina dye sequencing), the sequencing adapters may be P5 and P7 sequencing adapters (i.e. P5 at one end of the indexed PCR product, and P7 at the other). The indexing primer providing the P5 sequence may comprise the sequence of SEQ ID NO: 2. The indexing primer providing the P7 sequence may comprise the sequence of SEQ ID NO: 3.

Once formed, an indexed PCR product may be termed an “indexed DNA library sequence” or “indexed DNA fragment”. The pooled indexed PCR products, indexed DNA sequences, or indexed DNA fragments, may be termed an “indexed DNA library”.

### The Indexed DNA Library

The indexed DNA fragment sizes of the indexed DNA library may be filtered, such that only indexed DNA fragments of a desired or suitable length are available for sequencing. After the indexing PCR the indexed PCR fragments may be purified/isolated, for example by beads (e.g. SPRI beads). The purification may remove undesirable short fragments, primer-dimers or other PCR artefacts or reagents.

The indexed library may be checked for appropriate size distribution, We usually check the library size distribution, for example on tapestation or bioanalyzer instruments (Agilent), or similar. The size of the indexed DNA library may be adjusted by dilution after preparation for sequencing, for example to about 4 nM.

The indexed DNA library may be stored for later use, such as sequencing. For example, the DNA library may be stored frozen or chilled.

### Sequencing of the Indexed DNA Library

The indexed DNA library may be sequenced, or adapted to be sequenced. The sequencing may be next generation sequencing (NGS). The sequencing may be dye-sequencing (e.g. Illumina dye-sequencing), nanopore sequencing, or ion torrent sequencing. The skilled person will be familiar with a number of different sequencing technologies/methods that may be used, and the required sequencing adapters therefor.

The sequencing may be multiplexed sequencing, where multiple indexed DNA libraries are sequenced simultaneously.

### OTHER ASPECTS

Described herein is a method of preparing an indexed DNA library for sequencing of nucleic acid molecules the method comprising:
i) providing a multi-well array plate comprising rows and columns of reaction wells;
ii) providing nucleic acid molecules, wherein the nucleic acid molecules are distributed into a plurality of reaction wells on the multi-well array plate, such that there is no more than one single-stranded nucleic acid molecule from any given locus per reaction well,
iii) carrying out amplification of the nucleic acid molecule to provide multiple DNA copies of the nucleic acid molecule in each reaction well;
iv) fragmenting the DNA molecules of each reaction well and ligating a pair of looped adapters at each end or tagmenting using transposase-delivered adapters to form adapted-DNA fragments, wherein the looped adapters or transposase-delivered adapters comprise either a Column Index (Ci) sequence or a Row Index (Ri) sequence, wherein the Ci sequence is common to each looped adapter or transposase-delivered adapter of every reaction well in a column of the multi-well array plate, or wherein each Ri sequence is common to each looped adapter or transposase-delivered adapter of every reaction well in a row of the multi-well array plate;
vi) providing the indexed DNA library by performing indexing PCR on the adapted-DNA fragments, wherein the adapted-DNA fragments are amplified to form indexed PCR products using forward and reverse indexing primers, wherein either a Row Index (Ri) sequence or Column Index (Ci) sequence is introduced by each forward and reverse indexing primers onto each end of the adapted-DNA fragments, such that the resulting indexed PCR products comprise both a pair of flanking Column Index (Ci) sequences that are common to each well of a column and a pair of flanking Row Index (Ri) sequences that are common to each well of a row, and
optionally wherein the forward and reverse indexing primers further provide respective 5′ and 3′ sequencing adapters onto the indexed PCR products that are suitable for use in a sequencing reaction.

The nucleic acid may be DNA or RNA. In one embodiment, the nucleic acid is genomic DNA. In another embodiment, the nucleic acid may be mRNA.

Described is a method of preparing an indexed DNA library for whole genome sequencing of cells in a sample for the identification of single nucleotide variants, determining chromosome structural variations, or determining phasing information in the genome of the cells, the method comprising:
i) providing a multi-well array plate comprising rows and columns of reaction wells;
ii) providing genomic DNA of cells in a sample, wherein the genomic DNA is distributed into a plurality of reaction wells on the multi-well array plate, such that there is no more than one single-stranded genomic DNA molecule of any given locus per reaction well,
iii) carrying out whole genome amplification (WGA) of each genomic DNA molecule to provide multiple copies of the genomic DNA molecule in each reaction well;
iv) fragmenting the DNA molecules of each reaction well and ligating a pair of looped adapters at each end or tagmenting using transposase-delivered adapters to form adapted-DNA fragments, wherein the looped adapters or transposase-delivered adapters comprise either a Column Index (Ci) sequence or a Row Index (Ri) sequence, wherein the Ci sequence is common to each looped adapter or transposase-delivered adapter of every reaction well in a column of the multi-well array plate, or wherein each Ri sequence is common to each looped adapter or transposase-delivered adapter of every reaction well in a row of the multi-well array plate;
vi) providing the indexed DNA library by performing indexing PCR on the adapted-DNA fragments, wherein the adapted-DNA fragments are amplified to form indexed PCR products using forward and reverse indexing primers, wherein either a Row Index (Ri) sequence or Column Index (Ci) sequence is introduced by each forward and reverse indexing primers onto each end of the adapted-DNA fragments, such that the resulting indexed PCR products comprise both a pair of flanking Column Index (Ci) sequences that are common to each well of a column and a pair of flanking Row Index (Ri) sequences that are common to each well of a row, and
optionally wherein the forward and reverse indexing primers further provide respective 5′ and 3′ sequencing adapters onto the indexed PCR products that are suitable for use in a sequencing reaction.

The indexed nucleic acid may be sequenced, for example as described herein.

Therefore, also described is a method of whole genome sequencing of cells in a sample to provide data for the identification of single nucleotide variants (SNVs) in the genome of the cells, the method comprising:
i) preparing an indexed DNA library by carrying out the method according to the invention herein, or providing an indexed DNA library prepared in accordance with the method of the invention herein;
ii) sequencing the indexed DNA library to provide data for determining any single nucleotide variants (SNVs) in the genome of cells.

The sequencing data may be used to determine SNVs, for example as described herein. Additionally or alternatively, the sequencing data may be used to determine genetic changes relating to chromosome structural variations. The chromosome aberration may comprise a numerical and/or structural aberration.

Additionally or alternatively, the sequencing data may be used to determine phasing information in the cells.

The described methods may be provided in combination with determining somatic allele-specific copy number alterations (CNAs) according to the invention herein.

Advantageously, SNVs may be accurately determined in a sample of cells having high levels of CNAs.

The methods described herein may be used to monitor for, or detect, minimal residual disease (MRD) or cancer relapse. The sample may be from a subject having had previous cancer therapy, such as chemotherapy. The subject may be in a state of remission.

The invention may also include one or more features, either singularly or in combination, as disclosed in the description and/or in the drawings.

### Definitions

The term “spatially related cells” is understood to mean cells that are immediate neighbours to each other.

The term “false positive (FP) mutation” or “false positive (FP) SNV” is understood to mean a variant nucleotide that was not present in the genome prior to DNA extraction from intact cells, for example, a false mutation may be a damage-induced error or a replication error.

The terms “real mutation/SNV” or “true positive mutation/SNV” may be used interchangeably, and are understood to mean a variant nucleotide that is present in genomic DNA of living cells prior to DNA extraction.

The term “single nucleotide variant” (SNV) may include single nucleotide polymorphisms (SNPs), or any other variation in sequence, such as a mutation. Mutations or variations may include nucleotide substitutions, additions or deletions in a given sequence.

A “chromosomal aberration” is understood to be a missing, extra, or irregular portion of chromosomal DNA. It can be from a typical number of chromosomes or a structural abnormality in one or more chromosomes. They include a variety of aberrations such as deletions, duplications, and insertions. Balanced aberrations such as inversions and inter-chromosomal and intra-chromosomal translocations can occur. In addition, mobile element insertion, segmental duplications, multi-allelic chromosome numeric aberrations can occur. Final multiple combinations of the above can produce complex rearrangements.

“Phasing” is understood to be the task or process of assigning alleles (the As, Cs, Ts and Gs) to the paternal and maternal chromosomes. Phasing can help to determine whether matches are on the paternal side or the maternal side, on both sides or on neither side. Phasing can also help with the process of chromosome mapping—assigning segments to specific ancestors.

DNA that is “indexed to a well” means that the indexing of the DNA identifies the DNA from the well.

The skilled person will understand that optional features of one embodiment or aspect of the invention may be applicable, where appropriate, to other embodiments or aspects of the invention.

Embodiments of the invention will now be described in more detail, by way of example only, with reference to the accompanying drawings.

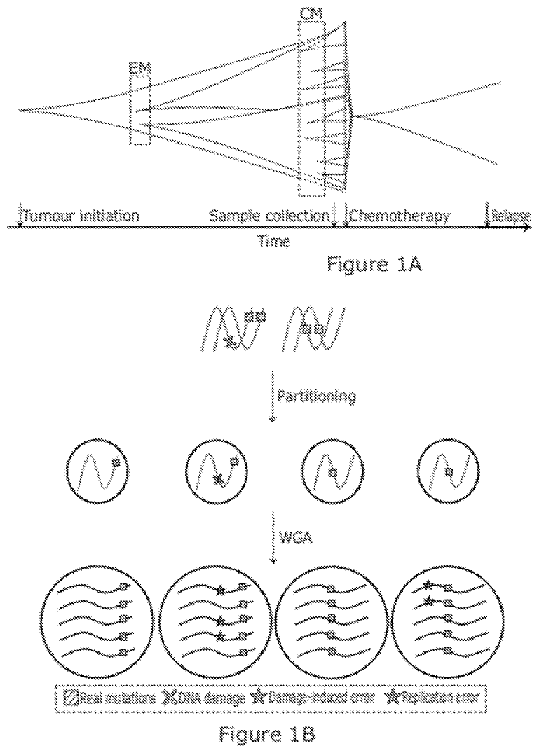

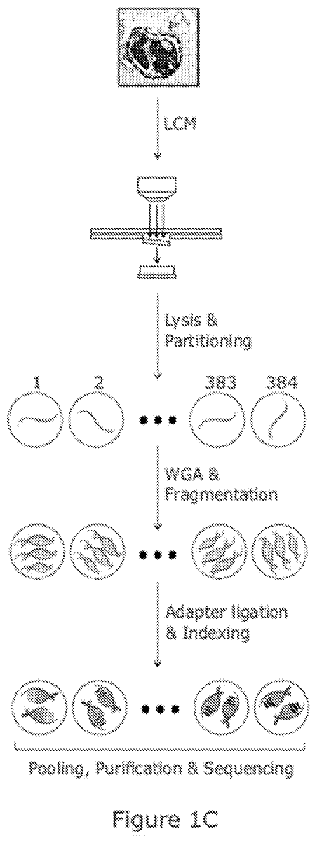

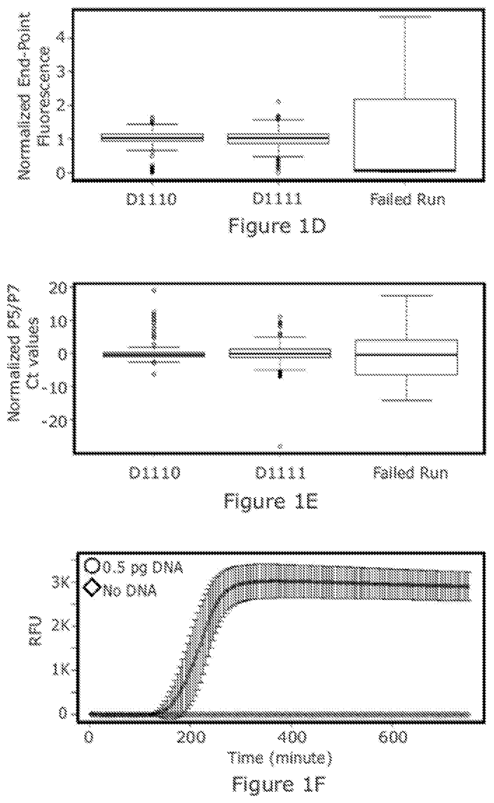

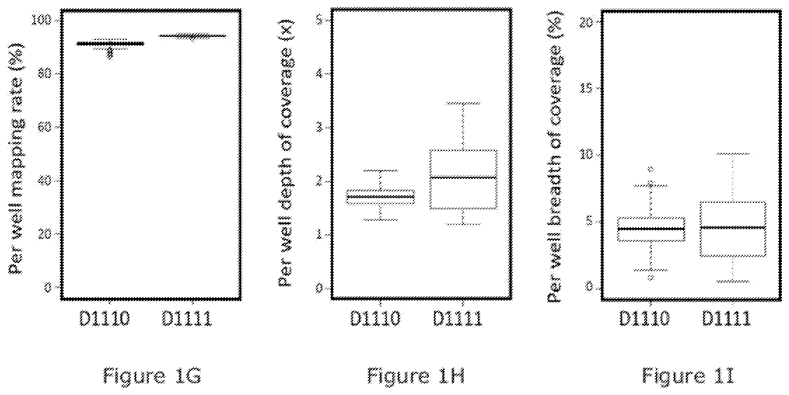

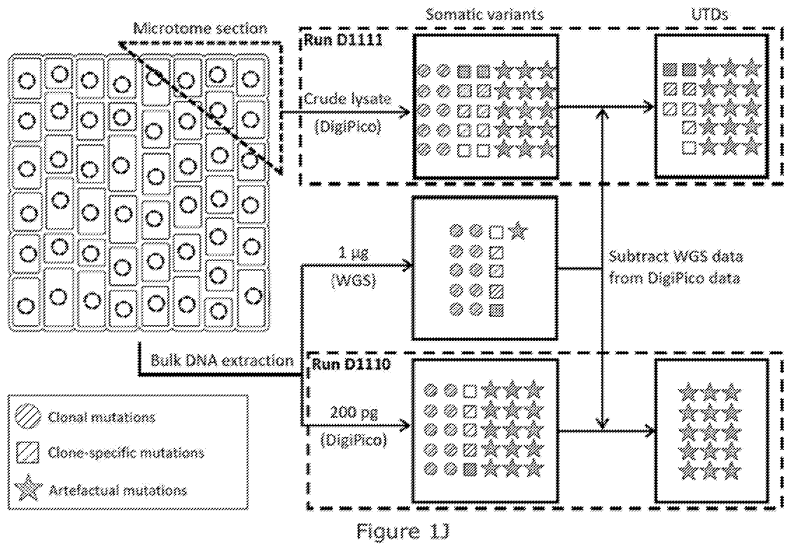

FIG. 1. DigiPico sequencing rationale, workflow, and performance. (A) WGS approaches can only identify early mutational processes (EM) in dominant expanded clones in a tumor. Currently active mutational processes (CM) result in a diverse set of sub-clones with different clone-specific mutations. This diversity determines the evolutionary trajectory of the tumor. (B) Template partitioning prior to WGA so that each compartment receives no more than one DNA molecule from each locus allows for the identification of artificial mutations. Since damage-induced and replication errors (stars) occur stochastically during replication, artefactual mutations result in dual-allelic compartments. Note that real mutations (squares) are always present in all product DNA strands within the same compartment. (C) DigiPico sequencing workflow. LCM: Laser-capture micro-dissection. (D) End-point relative fluorescent unit (RFU) from EvaGreen-labeled DNA was used to ensure homogeneous distribution of template and WGA process across the plate. RFU values were normalized to achieve a median of 1 in each run. (E) Per well qPCR using Illumina adapter primers (P5 and P7) measures the relative quantity of adapter-ligated products in each well. Ct values were normalized to achieve a median of 0 in each run. (F) Streamlining the DigiPico library preparation process required miniaturized a WGA that can specifically and sensitively amplify sub-picogram quantities of DNA in every well. Values represent the mean RFU values across 9 replicates. Error bars represent SD. (G, H, and I) preliminary analysis of DigiPico sequencing data from individual wells in each run confirms sequencing high-quality and homogeneity of mapping rate, depth of coverage and breadth of coverage as indicated. (J) Definition of unique to DigiPico (UTD) variants. Subtracting the SNVs that are identifiable in standard WGS data from corresponding DigiPico data results in UTD variants. These will mainly be consisted of artefactual mutations as well as some clone-specific mutations. Since the template in run D1110 is practically a subset of the template used for the standard WGS, all true variants in the DigiPico run D1110 are expected to also be present in the standard WGS data. In contrast, clone-specific variants in run D1111 are likely to be absent in the standard WGS data because of depth limitation, even though the DNA molecules supporting such variants might have been present in the bulk DNA sample at very low frequencies. In all boxplots the horizontal line represents the median. Boxes represent interquartile range (between the 25th and the 75th percentile). Whiskers represent the range excluding outliers. Outliers are defined as data points above or below 1.5 times the interquartile range.

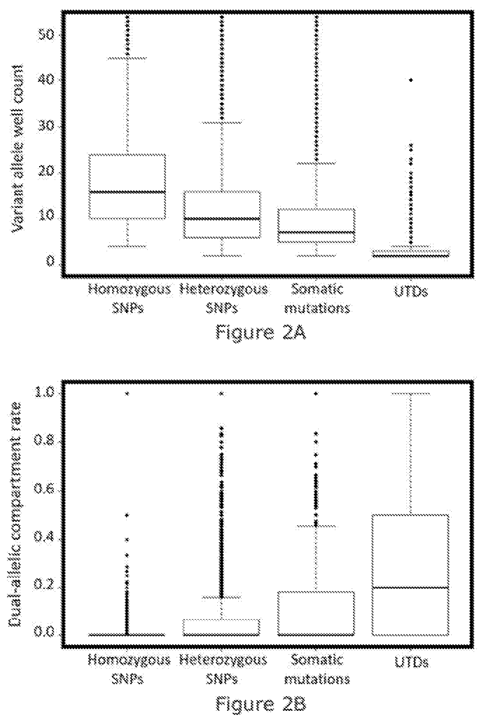

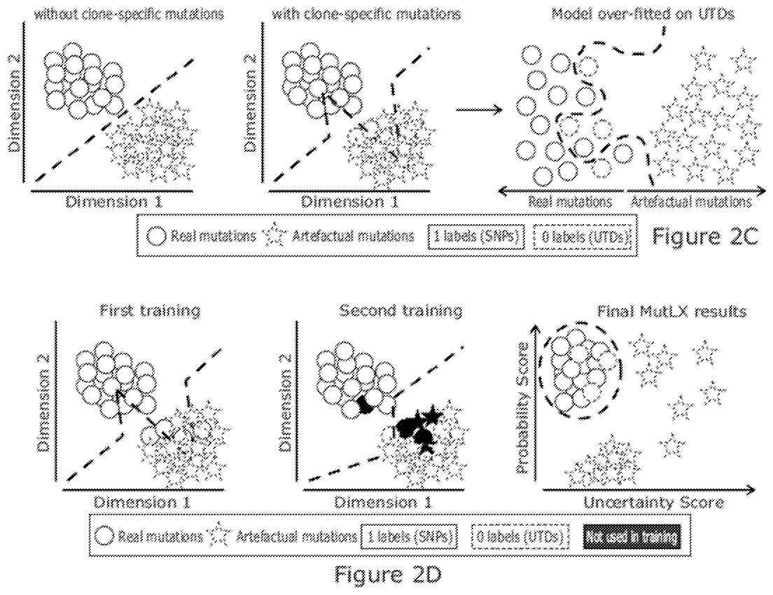

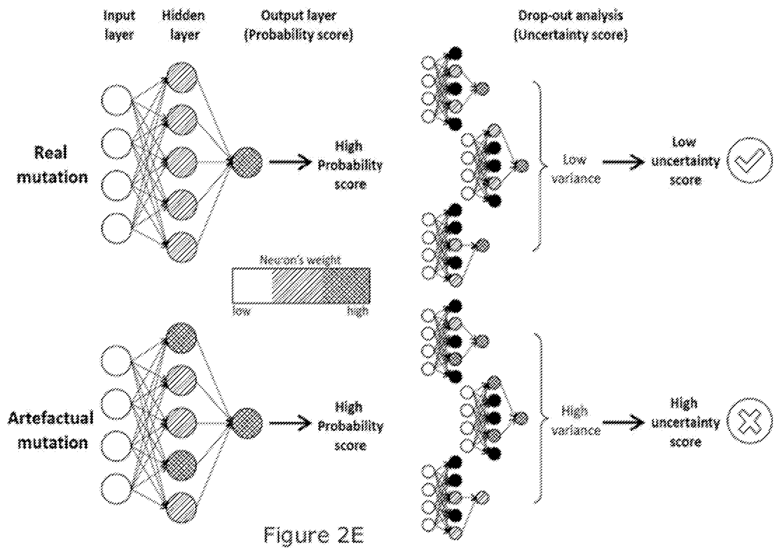

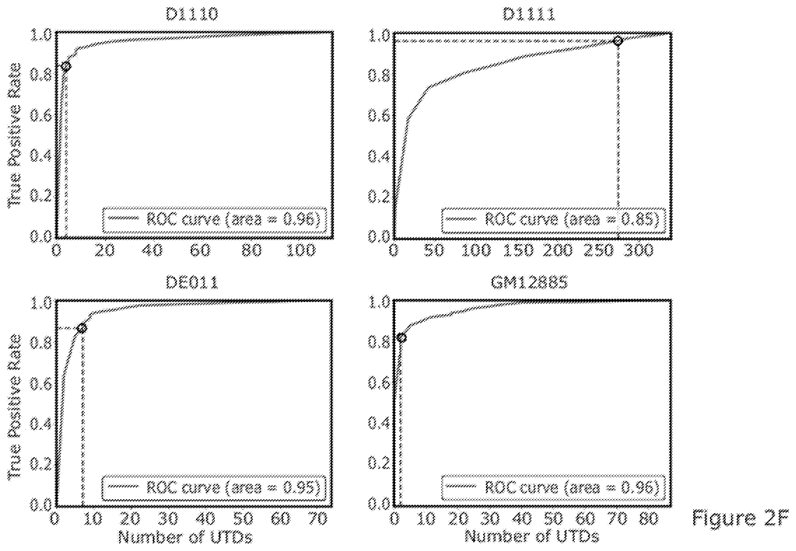

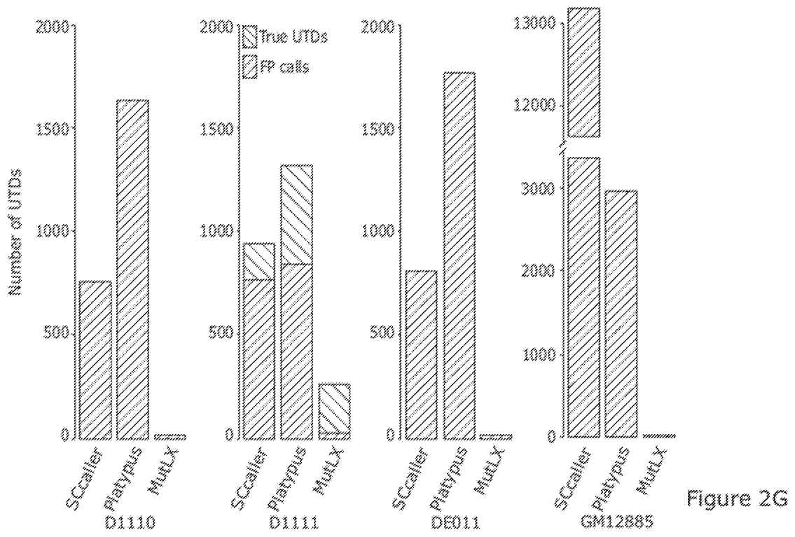

FIG. 2. Mono-allelic nature of DigiPico. (A) comparing the number of wells supporting various mutation types in run D1110 confirms that, as hypothesized, the majority of UTDs are present in only a few wells. Horizontal lines represent median. Boxes represent interquartile range. Whiskers show the range excluding outliers which are defined as being outside 1.5 times the interquartile range. (B) Similarly, the dual-allelic compartment rate of UTDs appears to be significantly higher when compared with true variants. This value was calculated by dividing the number of wells with co-presence of variant and reference alleles to the total number of wells with evidence for variant allele.

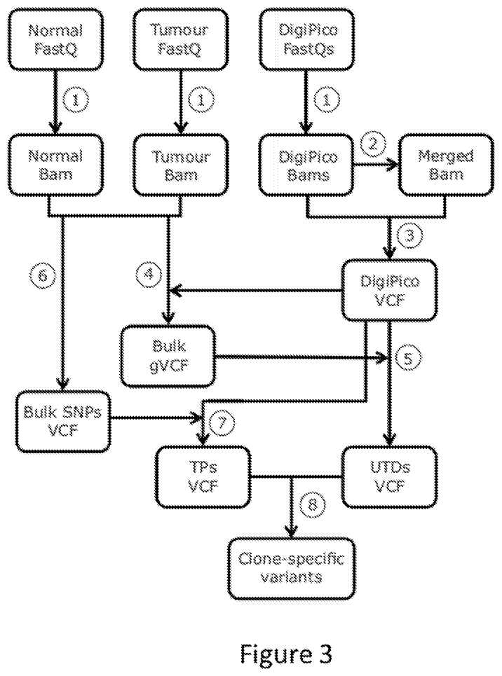

FIG. 3. Analysis workflow for DigiPico data for SNV identification. (1) Next generation sequencing reads from normal tissue, bulk of the tumor, and DigiPico library are first mapped to human genome to generate bam files. DigiPico reads are divided into 384 FastQ files, one for each well of the 384-well plate. (2) The 384 individual bam files from DigiPico are merged into a single bam file.

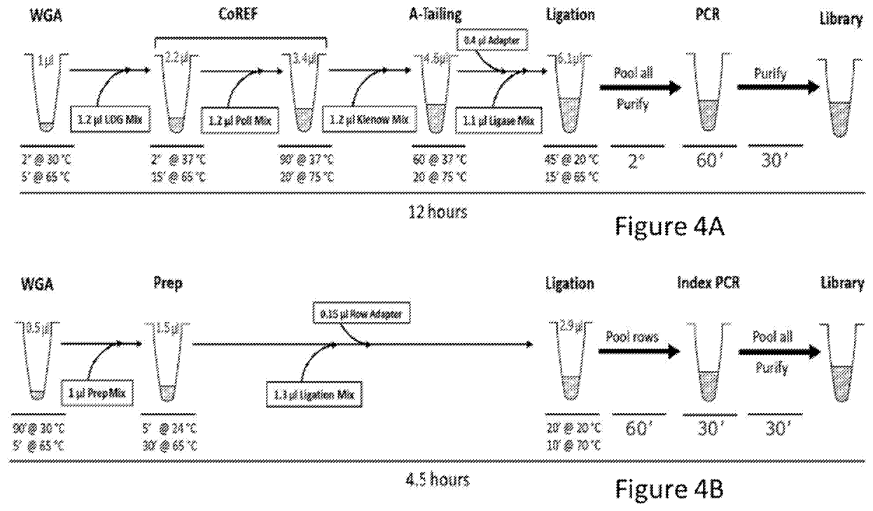

FIG. 4. Comparison of DigiPico and DigiPico2 workflows. (A) DigiPico workflow takes nearly 12 hours and is composed of 7 steps, 5 of which occur in a 384 well plate. (B) DigiPico2 workflow takes only 4.5 hours and is composed of 5 steps, only 3 of which occur in a 384 well plate. Reactions in blue occur in 384 well plate format, green in 16 wells, and orange in 1 tube.

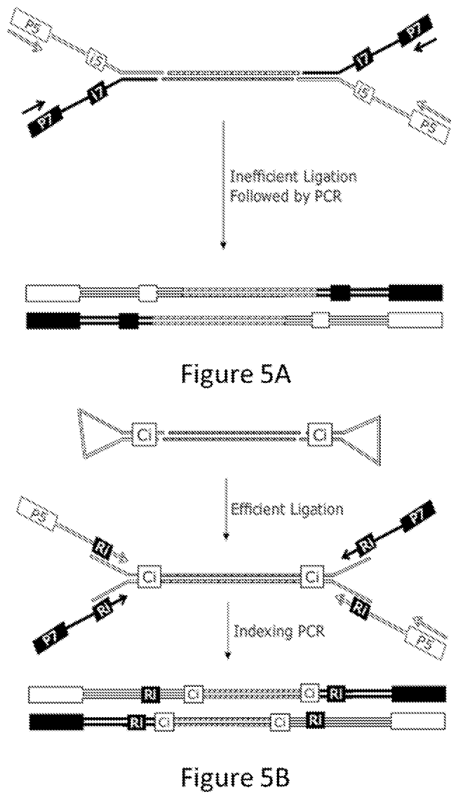

FIG. 5. Comparison of DigiPico and DigiPico2 indexing strategy. (A) Asymmetric ligation of 2 annealed indexing oligos introduces one i5 index and one i7 index. Combination of these indices can produce 384 different indices in DigiPico. Not that each strand receives one i5 index and one i7 index so no redundancy is present. (B) In DigiPico2 initially column indices (Ci) are introduced through efficient ligation of looped adapters. Next, Indexing PCR is performed using row indexing primers (Ri). Note that each strand receives the Ci and Ri index twice each which introduces redundancy needed for removal of index cross-contaminants.

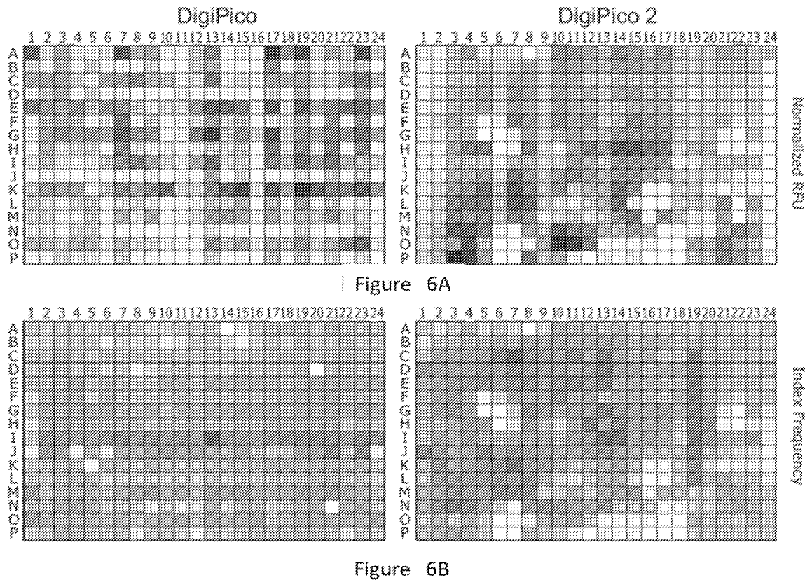

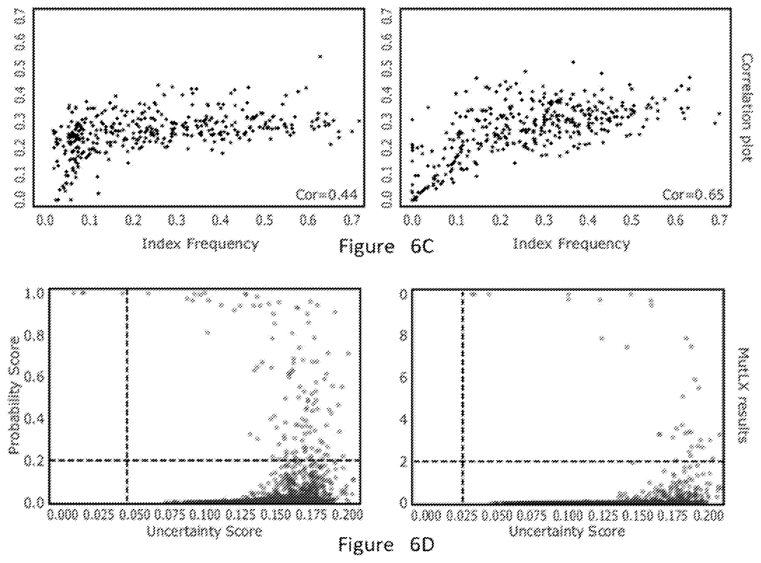

FIG. 6. Comparison of DigiPico and DigiPico2 results. (A) Both WGA products appear to be relatively homogenous across the plates. Values represent the relative fluorescence from measuring EvaGreen (RFU). (B) The frequency of indices from each well across the plates appears to correlate better in DigiPico2 with the RFU values of WGA products. (C) This fact can be quantified using a correlation plot.

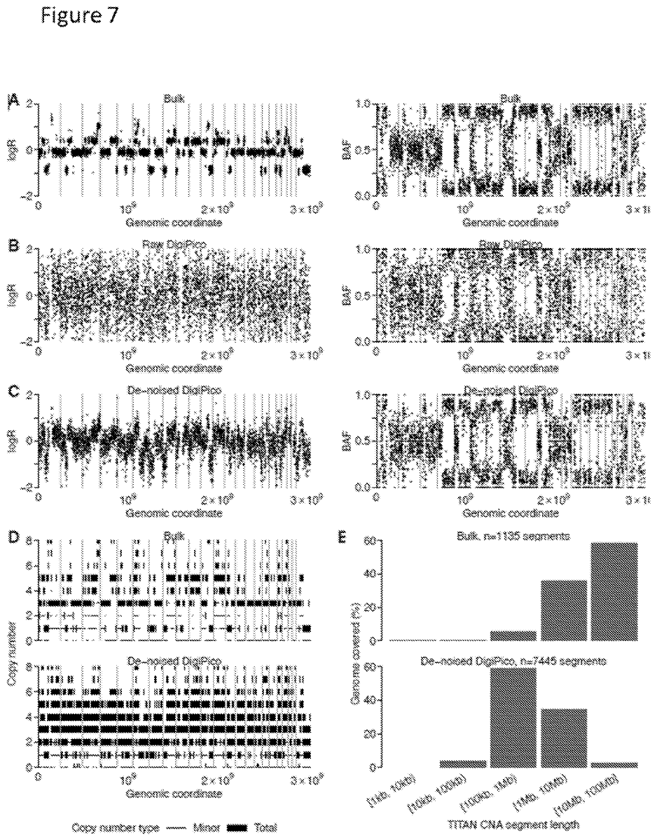

FIG. 7: DigiPico data de-noising for matched samples A-B1/A-D1. A. Whole genome logR (left) and BAF (right) profiles for bulk sample A-B1. LogR has been corrected for GC content and mappability as part of the standard TITAN pipeline. Vertical lines indicate chromosome ends. B. Raw logR and BAF profiles for DigiPico sample A-D1. C. De-noised logR and BAF profiles for DigiPico sample A-D1. D. TITAN CNA calls with default parameters for bulk sample A-B1 (top) and matched de-noised DigiPico data from sample A-D1 (bottom). E. Segment length distributions of TITAN CNA calls for samples A-B1 (top) and A-D1 (bottom).

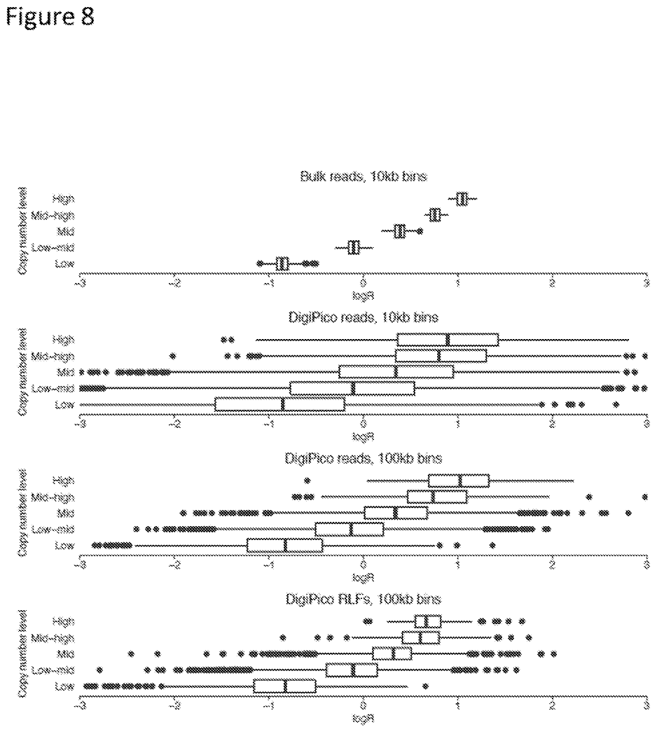

FIG. 8: De-noising of DigiPico logR data for matched samples A-B1/A-D1. Distributions of logR values for at different copy number states, determined manually from bulk data. As DigiPico data is cleaned (progressing from second panel to bottom panel), the overlap between the states is reduced.

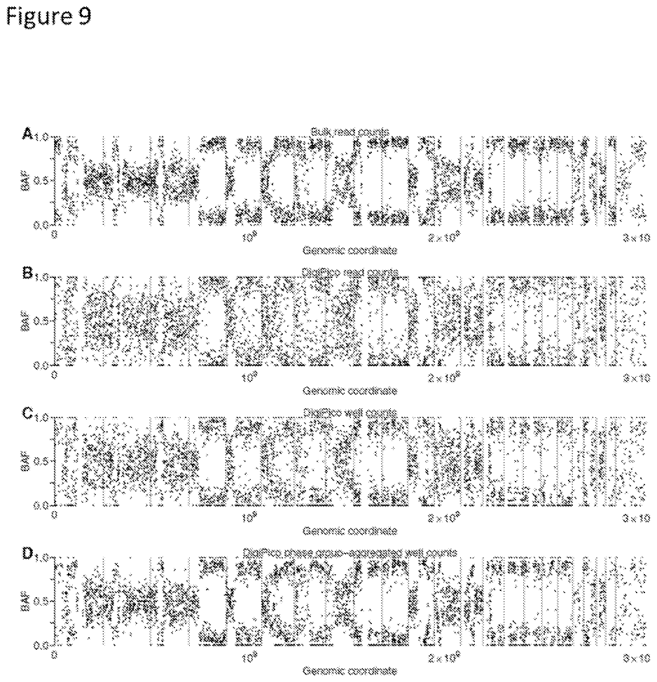

FIG. 9: De-noising of DigiPico BAF data for matched samples A-B1/A-D1. A. Bulk read count-derived BAF along the genome for sample A-B1. B. DigiPico read count-derived BAF for matched sample A-D1. C. DigiPico well count-derived BAF. D. DigiPico phase group-aggregated well count-derived BAF.

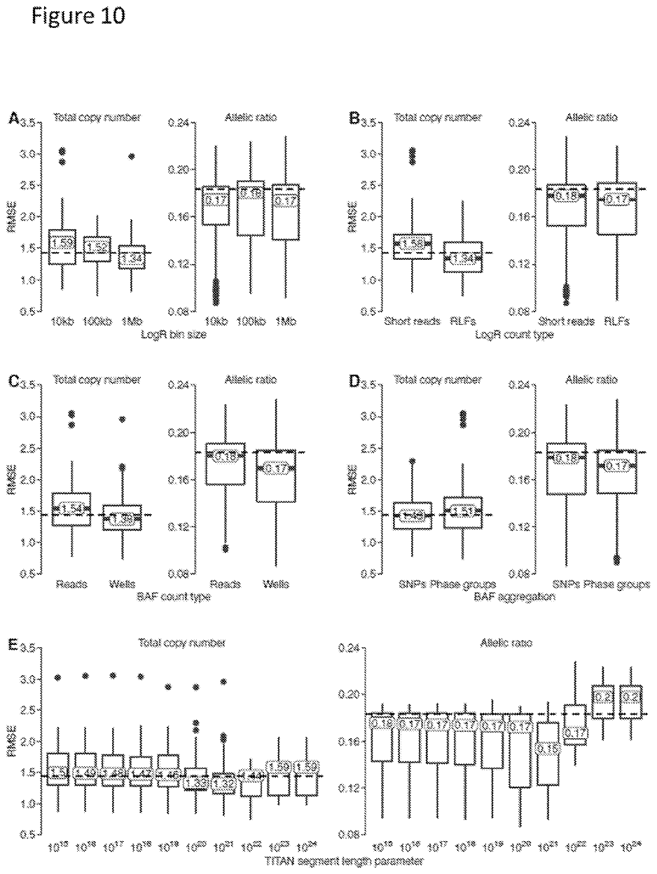

FIG. 10: CNA calls from the optimised PicoCNV algorithm. Whole genome PicoCNV CNA calling profiles for patients A (left) and B (right). Bulk TITAN calls are shown along the top with matched PicoCNV data vertically below.

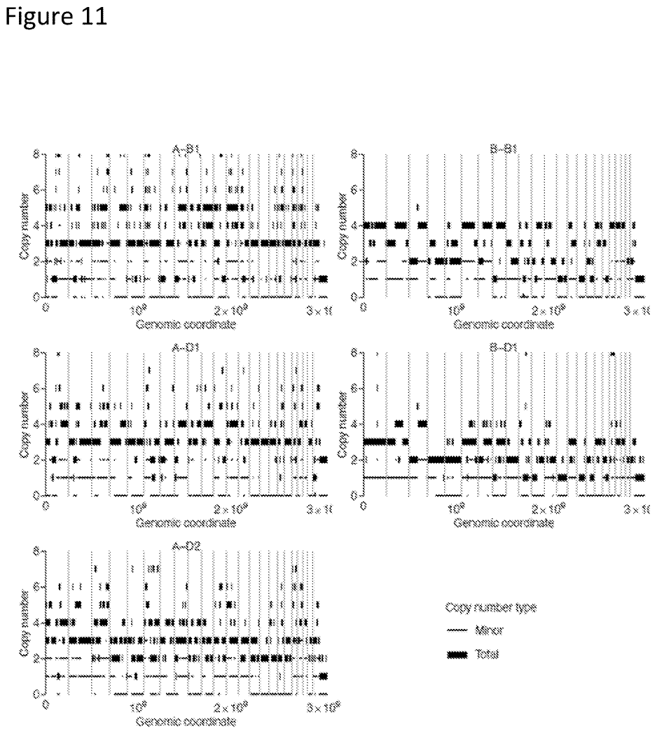

FIG. 11: PicoCNV optimisation marginal RMSE distributions. A. Marginal distributions of total copy number (left) and allelic ratio (right) RMSEs, controlling for logR bin size and varying the four other settings. The dotted horizontal lines indicate the RMSEs of CNA calls from the low-purity bulk sample A-B2 compared to sample A-B1. Marginal RMSE distributions controlling for logR count type (B), BAF count type (C), BAF aggregation (D) and TITAN expected segment length parameter (E).

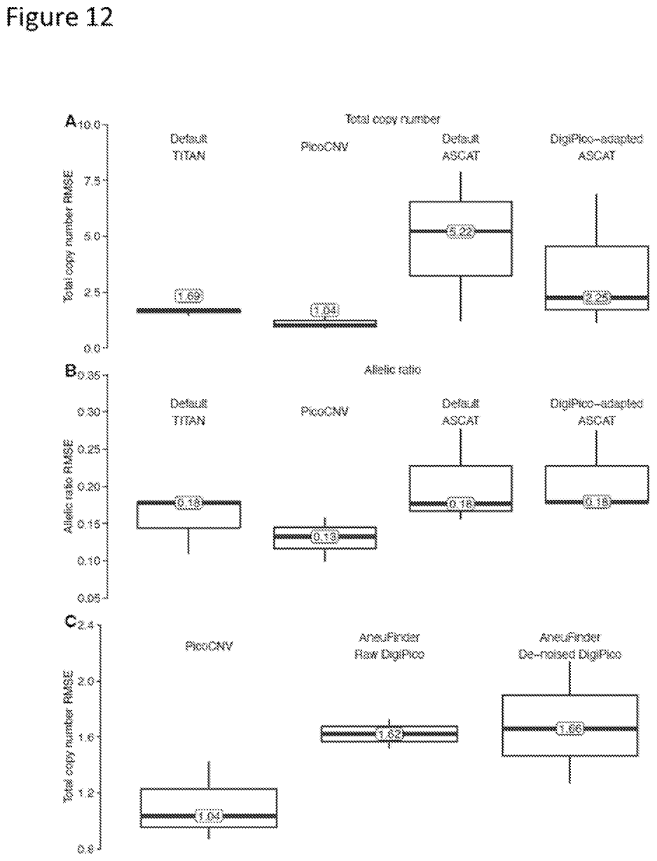

FIG. 12: Benchmark of PicoCNV against other methods. Copy number was called from DigiPico data using the TITAN and ASCAT algorithms with both default and DigiPico-adapted pipelines. The RMSEs between DigiPico calls and bulk calls from the same algorithms are shown in terms of total copy number (A) and allelic ratio (B). C. Total copy number RMSEs comparing DigiPico calls to bulk TITAN calls, using the PicoCNV and AneuFinder algorithms.

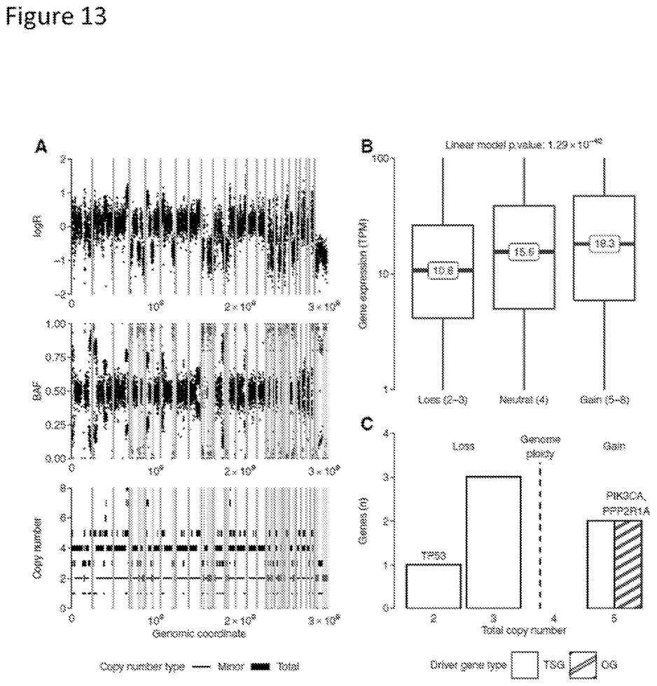

FIG. 13: Application of PicoCNV to MRD. A. Genome tracks of logR (top), BAF (middle) and PicoCNV CNA calls (bottom) for sample C-D1. Highlighted regions in the BAF and CNA call tracks indicate regions of LOH, totalling 22.9% of the genome. B. Distribution of gene expression by gene copy number state. Only expressed genes (TPM>1) are shown. The genome ploidy was 3.8, so genes with copy number four were considered to be copy number neutral. C. CNAs of tumour suppressor genes (TSGs, clear) and oncogenes (OGs, striped). Copy number neutral driver genes are not shown.

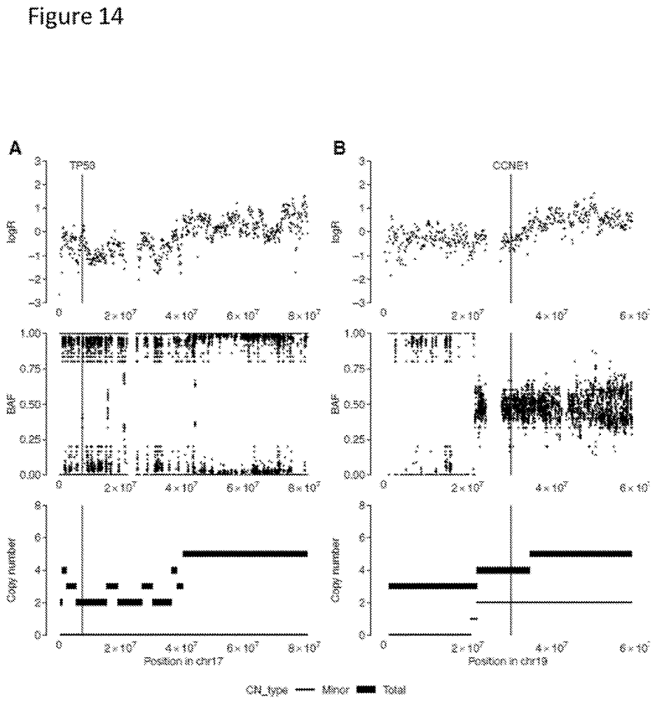

FIG. 14: Copy number data for selected driver genes in MRD. Chromosome-wide tracks of logR (top), BAF (middle) and PicoCNV CNA calls (bottom) for chromosomes 17 (A) and 19 (B). The vertical lines indicate the loci of TP53 (A) and CCNE1 (B).

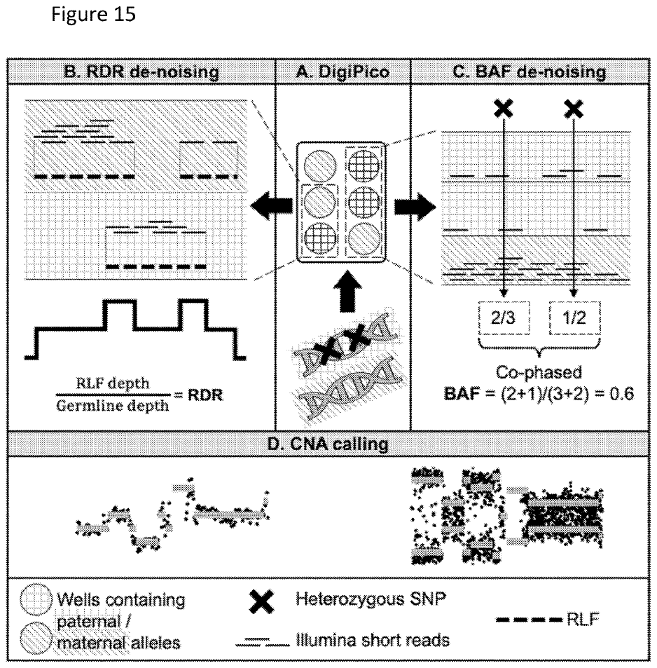

FIG. 15: PicoCNV pipeline overview
A. Large fragments of tumour DNA are distributed into wells such that each genomic locus is covered by at most one fragment per well, before being sequenced.
B. Co-local short reads within each well are combined to create reconstructed large fragments (RLFs). Calculating the RDR from the RLF depth counteracts variations in the Illumina short read sequencing depth between fragments.
C. Nearby SNPs supported by overlapping sets of wells come from the same allele. Calculating the BAF from well counts for haplotype blocks counteracts variations in sequencing depth.
D. The de-noised RDR and BAF data are used to call allele-specific copy number alterations.

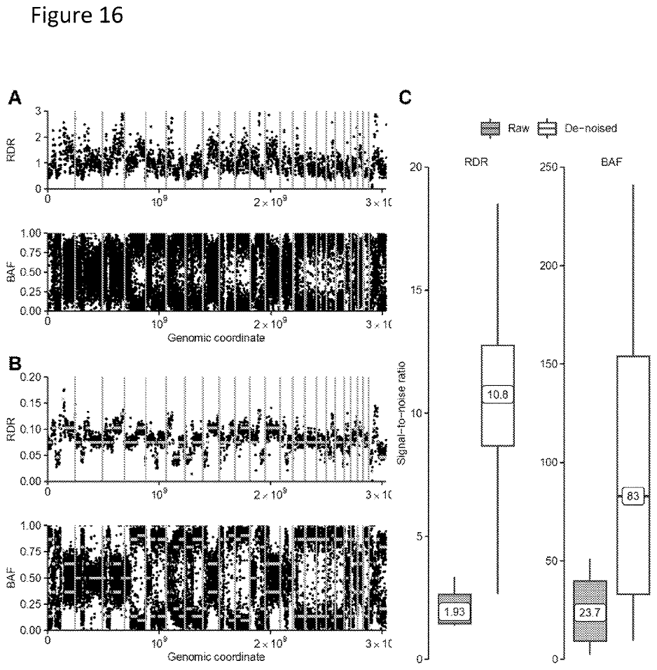

FIG. 16: PicoCNV data de-noising
A. Raw RDR (top) and BAF (bottom) tracks for the DigiPico sample from patient 11152, derived from Illumina short read sequencing depths. RDR values were calculated in non-overlapping 1 Mb windows and corrected for GC content and mappability. BAF values were calculated at heterozygous SNPs.
B. De-noised RDR and BAF tracks for the DigiPico sample from patient 11152. Coloured segments indicate copy number states fitted by PicoCNV.
C. Per-patient signal-to-noise ratios (SNRs) for the RDR (left) and BAF (right). To calculate signal strength, data points in the copy number states {1,1} and {1,0} according to bulk ASCAT calls were compared. The signal was the squared difference in the means of these two sets. The noise was measured as the average within-segment variance, using segments taken from bulk ASCAT calls.

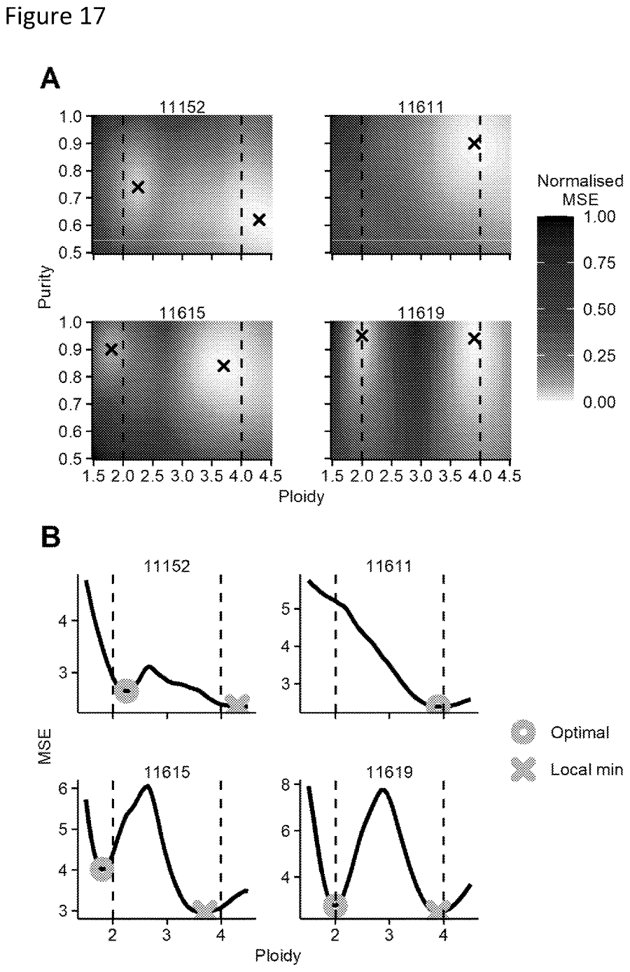

FIG. 17: Purity/ploidy grid search
A. Whole-genome MSE values for each purity/ploidy value pair. Within each patient, the range of MSE values was min-max normalised for display purposes. Vertical dotted lines indicate the location of diploid and tetraploid solutions.
B. Illustration of ploidy selection. For each ploidy value, the optimal purity according to MSE was selected to create a 1-dimensional selection problem. Local minima on the resulting curve were then identified. For curves with two minima, their heights relative to the interposing maximum were calculated. If the height of the first minimum was at least one half the height of the second, then the first minimum was selected as the sample ploidy.

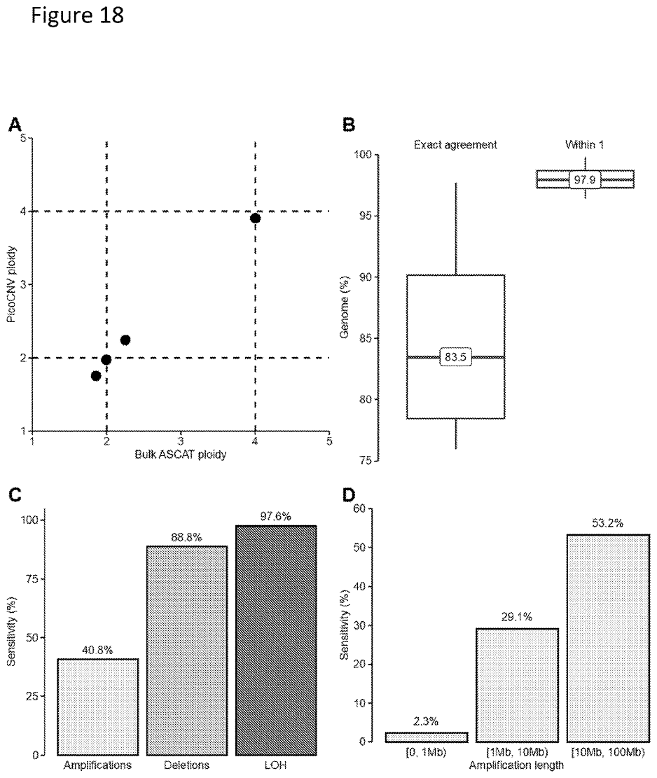

FIG. 18: Evaluation of PicoCNV performance
A. Comparison of true ploidy values (mean total copy number) obtained from bulk ASCAT and PicoCNV. Dashed lines indicate diploid and tetraploid solutions. The diploid solution for patient 11619 bulk ASCAT is shown.
B. Per-patient accuracies of PicoCNV relative to bulk ASCAT.
C. Sensitivity of PicoCNV to detect amplifications, deletions and LOH.
D. Sensitivity to detect amplifications, stratified by amplification length.

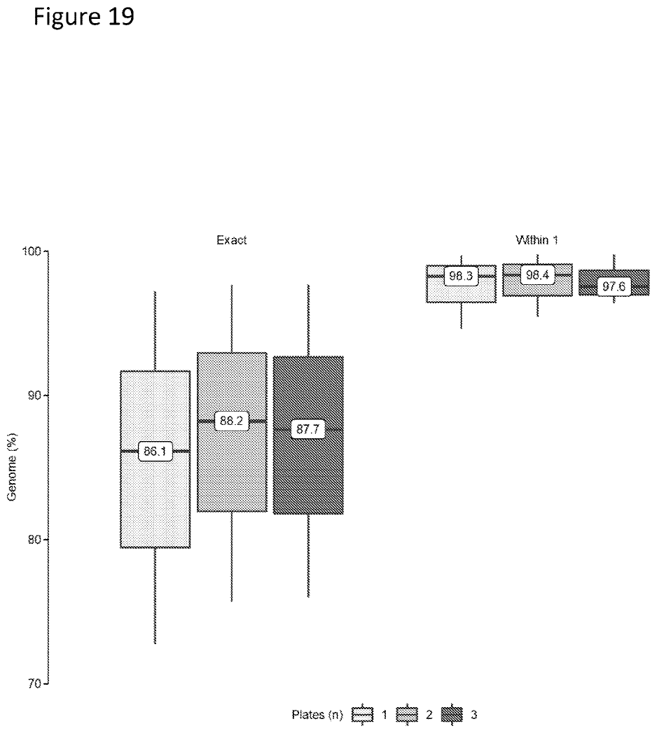

FIG. 19: Sub-sampling effect on PicoCNV accuracy Distributions of per-patient percentages of the genome where PicoCNV and bulk ASCAT copy number states agree exactly (left) and are within one of each other (right). For both levels of accuracy, results using one, two and three plates are shown from left to right. Data are from patients 11611, 11615 and 11619, for whom matched bulk samples were available and DigiPico sequencing used three plates in total.

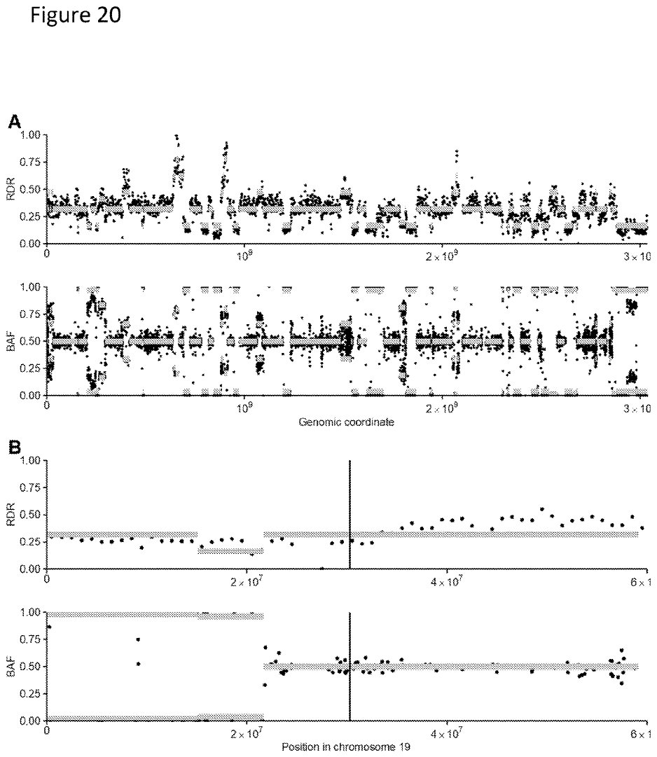

FIG. 20: Application of PicoCNV to MRD
A. De-noised RDR (top) and BAF (bottom) tracks for the MRD sample. Coloured segments indicate copy number states fitted by PicoCNV. Highlighted regions of the genome were determined to have undergone LOH.
B. RDR and BAF tracks for chromosome 19, with fitted PicoCNV copy number states. The vertical line indicates the position of the CCNE1 gene.

### EXAMPLE 1—DEVELOPMENT OF THE DIGIPICO LIBRARY PREPARATION METHOD FOR THE MUTATIONAL ANALYSIS OF PICOGRAM QUANTITIES OF DNA

### Summary

Bulk whole genome sequencing (WGS) enables the analysis of tumour evolution but, because of depth limitations, can only identify old mutational events. The discovery of current mutational processes for predicting the tumour's evolutionary trajectory requires dense sequencing of individual clones or single cells. Such studies, however, are inherently problematic because of the discovery of excessive false positive mutations when sequencing picogram quantities of DNA. Data pooling to increase the confidence in the discovered mutations, moves the discovery back in the past to a common ancestor. Here we report a robust whole genome sequencing library preparation method (DigiPico) that virtually eliminates all false positive results while retaining an excellent proportion of true positives. Overall, we propose the DigiPico method as a powerful framework for the identification of clone-specific variants at an unprecedented accuracy.

### Introduction

In this work we developed a single DNA molecule WGA and sequencing approach to obtain high-quality and data-rich sequencing results from picogram quantities of DNA obtained from clinical samples (we termed DigiPico: for Digital sequencing of Picograms of DNA).

### Material and Methods

### Patient Samples and Consent

Patient #11152 provided written consent for participation in the prospective biomarker validation study Gynaecological Oncology Targeted Therapy Study 01 (GO-Target-01) under research ethics approval number 11/SC/0014. Necessary informed consents from study participants were obtained as appropriate. Blood samples were obtained on the day of surgery. Tumour samples were biopsied during laparoscopy or debulking surgery and were immediately frozen on dry ice. All samples were stored in clearly labelled cryovials in −80° C. freezers.

### Sectioning and LCM

Frozen tumour samples were embedded in OCT (NEG-50, Richard-Allan Scientific) and 10-15 μm sections were taken using MB DynaSharp microtome blades (ThermoFisher Scientific) in a CryoStar cryostat microtome (ThermoFisher Scientific). Tumour sections were then transferred to PEN membrane glass slides (Zeiss) and were immediately stained on ice (2 minutes in 70% ethanol, 2 minutes in 1% Cresyl violet (Sigma-Aldrich) in 50% ethanol, followed by rinse in 100% ethanol. A PALM Laser Microdissection System (Zeiss) was used to catapult individual tumour islets into a 200 μl opaque AdhesiveCap (Zeiss).

### Standard WGS and Data Analysis

DNA was extracted using DNeasy blood and tissue kit (Qiagen). Up to 1 μg DNA was diluted in 50 μl of water for fragmentation using a Covaris S220 focused-ultrasonicator instrument to achieve 250-300 bp fragments. The resulting DNA fragments were then used for library preparation using NEBNext Ultra II library preparation kit (NEB), following the manufacturer's protocol. The resulting libraries were sequenced on Illumina NextSeq or HiSeq platforms at a depth of 30-40× over human genome. Sequencing reads in the FastQ format were initially trimmed using TrimGalore (14) and were then mapped to human hg 19 genome using Bowtie2 (15). Germline variant calling was performed using GATK's HaplotypeCaller (16). Somatic variants were called using Strelka2 with a variant allele fraction cut-off of 0.2 (17).

### DigiPico Sequencing

200 μg of purified DNA, 20-30 resuspended nuclei, or laser-capture micro-dissected tumour islets, were first denatured using 5 μl of D2 buffer from Repli-g single cell kit (Qiagen). After 5 minutes incubation at room temperature, 95 μl of water was added to the sample and then using Mosquito HTS liquid handler (TTP Labtech) 200 nl of the denatured template was added to each well of a 384-well reaction plate already containing 800 nl of WGA mix (0.58 μl Sc Reaction Buffer, 0.04 μl Sc Polymerase (REPLI-g Single Cell kit, Qiagen), 0.075 μl 1 mM dUTP (Invitrogen), 0.04 μl EvaGreen 20× (Biotium), and 0.065 μl water). The plate was incubated at 30° C. for 2 hours followed by heat inactivation at 65° C. for 15 minutes. Addition of EvaGreen in the reaction allows for monitoring of the WGA reaction using a real-time PCR machine if required (18). Next, controlled enzymatic fragmentation (19) reaction steps were sequentially performed on the whole genome amplified DNA without any purification steps. Briefly, (A) 1200 nl of UDG mix (0.08 U/μl rSAP (NEB), 0.2 U/μl UDG (NEB), 0.4 U/μl EndoIV (NEB) in 1.8× NEBuffer 3) was added with 2 hours incubation at 37° C. and heat inactivation at 65° C. for 15 minutes. (B) 1200 nl of Poll mix (0.4 U/μl DNA Polymerase I (NEB) 0.25 mM dNTP, 8 mM MgCl2, and 0.8 mM DTT) was added with 1.5 hours incubation at 37° C. and heat inactivation at 70° C. for 20 minutes. (C) 1200 nl of Klenow mix (0.5 U/μl Klenow exo-(NEB), 0.5 mM dATP, 8 mM MgCl₂, and 0.8 mM DTT) was added with 45 minutes incubation at 37° C. and heat inactivation at 70° C. for 20 minutes. (D) 400 nl of 20 μM full-length Illumina adapter oligos (Table S1) with well specific indices were added to each well followed by the addition of 1100 nl of Ligation mix (40 U/μl T4 DNA Ligase (NEB), 5 mM ATP, 11.5% PEG 8000 (Qiagen), and 6.8 mM MgCl2) and with 30 minutes incubation at 20° C. and heat inactivation at 65° C. for 15 minutes.

The resulting products were then pooled and the DNA was precipitated using an equal volume of isopropanol. DNA was then resuspended in water and the products were dual-size selected using Agencourt AMPure XP SPRI magnetic beads (Beckmann coulter) with 0.45× bead ratio for the left selection and an additional 0.32× for the right selection. The purified DNA was then resuspended in water and was immediately used for limited-cycle PCR amplification using the P5 and P7 primer mix (Table S1). PCR was performed for 12 cycles with 10 seconds annealing at 55° C. and 45 seconds extension at 72° C. Final products were bead purified at 0.9× ratio. The resulting libraries were then sequenced on Illumina sequencing platforms in 2×150 paired-end sequencing mode to achieve a depth of coverage of 30-40× over human genome. The additional processing steps required for the DigiPico library preparation, at present, adds nearly £250 to the total reagent costs.

### Analysis of DigiPico Sequencing Data

The analysis pipeline of DigiPico sequencing data is presented in FIG. 3. Briefly, 384 paired-read FastQ files for each well were obtained after demultiplexing the Illumina sequencing data. FastQ files were trimmed for adapter sequences and quality (14). The first 12 nucleotides of each read were also removed. Trimmed reads were mapped to human hg19 reference genome using Bowtie2 (15) with the ignore-quals parameter activated and duplicate reads were marked using Picard Tools (20). Joint variant calling was then performed on all 384 individual bam files together with a merged bam file from all wells using Platypus variant caller (21). Next, all low-quality variants were removed by applying the quality filters (QUAL>60, FR>0.1, HP≤4, QD>10, and SbPval≤0.95). Moreover, the total number of wells covering each locus (Tw) and the number of wells supporting each variant (Vw) were determined and the well count filters (Tw>5, Vw>2, and Vw/Tw>0.1) were applied to only retain the high confidence loci for analysis. Lastly, all regions of the genome with bad mappability (22) were removed from the analysis using VCFtools (23). The resulting list of high confidence de novo DigiPico variants were then used to perform variant re-calling (genotyping) on WGS data from blood and bulk of the tumour using Platypus with minPosterior parameter set to 0 and minMapQual parameter set to 5. Any variant that was confidently unsupported in both of the standard WGS data was extracted as a UTD (Unique to DigiPico) variant. Any variant that was confidently also present in the bulk sequencing data of the blood sample (based on GATK analysis) was extracted as a TP (True Positive) variant (FIG. 3).

### Results

### Implementation of DigiPico Sequencing Approach

A key feature of amplification errors with or without prior DNA damage is that they are introduced at random during the amplification process (6, 7, 28). We, therefore, hypothesized that when amplifying and sequencing a single DNA molecule an artefactual mutation would be present in only a fraction of the reads that have resulted from sequencing the original single DNA molecule. In contrast, genuine variants would be expected to be present in all such reads. Partitioning of the template DNA into individual compartments prior to WGA, such that each compartment receives no more than one DNA molecule from every locus would result in such single DNA molecule sequencing data (FIG. 1B). Since the artefactual mutations would result in compartments with reads supporting multiple alleles this approach enables the identification of these artefacts and allows for the elimination of FP variant calls. In addition, such partitioning approach also results in the independent progression of WGA reactions for each locus. Thus, providing multiple internal replication data for the WGA process. While genuine variants are expected to be regularly present across replicates, the artefactual mutations, because of their stochastic nature, will likely have a limited presence in a small number of compartments. Consequently, taking both of these points into account, such a WGA and sequencing approach can result in distinctive distribution patterns across the compartments for artefactual mutations compared to real mutations. Machine learning methods can then be applied to distinguish real from artefactual mutations. While previous partitioning and sequencing methods have been described to obtain haplotype information, there is no such method for distinguishing true mutations from artefactual mutations (19, 29, 30).

To fully benefit from the data-richness of a partitioning and sequencing approach for accurate genomics study of clinical samples, we developed DigiPico sequencing (FIG. 1C). To perform DigiPico sequencing, first, we uniformly distribute nearly 200 μg of DNA (obtained from 20-30 human cells) into individual wells of a 384-well plate. This ensures that the likelihood for the co-presence of two different DNA molecules from the same locus in the same well is less than 10% (19). Following WGA, each well is then processed independently into indexed libraries, each receiving a unique barcode sequence, prior to pooling and sequencing (FIG. 1C). Since, in our approach, the key distinguishing factor for artefactual mutations lies in their peculiar distribution pattern, homogeneous distribution and amplification of DNA molecules as well as consistent depth of sequencing coverages across the wells would be critical. Achieving this homogeneity ensures that the differences in the distribution pattern of true and artefactual mutations are maximized. To ensure that the required homogeneity is achieved, during every DigiPico library preparation we monitored the WGA reactions' progress and quantified the final outcome for all the wells. The former was achieved by adding EvaGreen dye to the WGA reactions and monitoring the fluorescent intensity every 5 minutes in real-time. EvaGreen is an intercalating dye that binds to the minor groove of the DNA and therefore, does not interfere with the isothermal WGA reactions (18). For the latter, we introduced a per-well qPCR step to measure the relative number of adapter-ligated fragments in each well using adapter specific primers prior to pooling. Only libraries that passed both of these homogeneity tests were used for sequencing (FIGS. 1D and 1E). Importantly, we also miniaturized the WGA reaction volumes to 1 μl. This could only be achieved after the identification of a compatible multiple displacement amplification (MDA) approach. Comparing six different MDA strategies, REPLI-g Single Cell amplification was the only method that met the required sensitivity and selectivity for our purpose (FIG. 1F). Reaction miniaturization allowed us to streamline the library preparation process in a single 384-well plate without the need for intermediate purification steps using readily available automated pipetting instruments. Finally, we aimed to optimize the DigiPico library preparation process for frozen clinical samples. This was achieved by performing the WGA reaction directly on the crude lysate of small groups of neighbouring cells (tumour islets) isolated via LCM (laser-capture micro-dissection). This strategy ensured the minimal loss of genomic material while minimizing the manipulation time and thereby, reducing the chance of template oxidation.

DigiPico Sequencing Platform Generates High Quality Libraries from Limited Clinical Samples

Having optimized all the necessary aspects of DigiPico library preparation process, we decided to assess the quality of DigiPico libraries obtained from clinical samples. For this purpose, we prepared DigiPico libraries D1110 and D1111 from a frozen recurrent tumour sample (PT2R) obtained from a high-grade serous ovarian cancer patient (#11152). In this experiment, while D1110 library was prepared from 200 pg of template taken from a bulk DNA extraction of the PT2R sample, the D1111 library preparation was directly performed on a small frozen section of the remainder of this tumour sample (containing nearly 30 cancer cells). Each library was sequenced on an Illumina NextSeq platform to obtain nearly 400,000,000 reads in 150×2 paired-end format. The initial assessment of the obtained sequencing data revealed that both the D1110 and D1111 libraries have resulted in high quality sequencing data with an overall mapping rate of 91.35% and 94.27% on human hg19 genome, respectively (FIG. 1G). Analysing the homogeneity of distribution across the plates indicated that in runs D1110 and D1111, on average, each well covers nearly 4.4% and 4.6% of the genome with an average depth of 1.7× and 2.1× in each well, respectively, with an outstanding homogeneity across the plates (FIGS. 1H and 1I). This cumulatively resulted in a breadth of coverage of 92.1% and 91.1% with a depth of 30× and 43× for each run, respectively. These results confirmed that DigiPico sequencing can be used to produce high-quality sequencing data with excellent coverage from limited amount of frozen clinical samples.

Lastly, we assessed whether our initial hypotheses regarding the distinctive distribution pattern of different mutation types hold true in actual DigiPico datasets. For this purpose, we assumed that any variant that is shared between a DigiPico dataset and the standard bulk sequencing data of the same tumour sample must be a true variant. These should mainly consist of germline SNPs and clonal somatic variants. As a result, by definition, all FP variant calls and the majority of clone-specific mutations (had they existed in the sample under study) will be among variants that are only present in the DigiPico data and not in the bulk WGS data. These variants are referred to as UTD (Unique to DigiPico) for simplicity, hereafter. Consequently, given that the standard bulk sequencing data of the PT2R sample had been obtained from the same DNA extract that was used for D1110 library preparation, nearly all the UTD variants in D1110 DigiPico run ought to be artefacts (FIG. 1J). To the contrary, the UTDs in run D1111 are likely to contain some clone-specific mutations alongside the artefactual mutations (FIG. 1J). Therefore, we used the UTD variants in run D1110 as a representative of artefactual mutations in our analysis. Comparing the frequency of wells with co-presence of two allele for the same locus (FIG. 2A) as well as the number of wells supporting each variant (FIG. 2B) in run D1110 showed that UTDs had significantly higher proportion of the former and lower number of the latter compared to any other category of mutations (p<2e-16 for both analyses, One-way ANOVA followed by Tukey HSD testing). This clearly supported the distinct distribution pattern of artefactual mutations in this DigiPico dataset as hypothesized.

### DISCUSSION

In this work we presented DigiPico as a platform for the identification of mutations from small groups of cells with unprecedented accuracy on a whole genome scale. We believe that this work provides an important stepping stone for the discovery of current or recent somatic mutational processes that occur in cancer and normal tissue. Understanding current mutational processes is key for predicting the evolutionary trajectory of a tumour and, potentially, for interfering with such trajectories therapeutically. A mutation that is identified in bulk sequencing of a tumour must have occurred at a point during the extended history of a tumour from the initiation till presentation. In contrast, a cell-specific mutation must have occurred during the limited lifetime of that cell. Similarly, a mutation in a small clone that has been derived from a single cell is also recent. The age of such a mutation can't be more than the age of the clone which is defined by the number of cell divisions it took to generate that clone. Studying patterns in cell-specific or small-clone-specific mutations can allow for the identification of recent or current mutational processes (1). Defining such processes is highly desirable since they can be causally linked to biological or chemical phenomena and, therefore, yield significant mechanistic insights. Identifying these mechanisms have important practical implications since they are potentially amenable for therapeutic intervention or for predicting future tumour behaviour. The current state of the art does not allow the direct accurate identification of mutations from individual cells or individual small clones from tumours. DigiPico will enable this endeavour for the first time.

To overcome significant technical pitfalls predominantly related to the discovery of false positive mutations, current methods for single cell WGS analysis either require extensive validation studies (11) or rely on combining data from multiple cells to obtain reliable mutations that are shared between cells (12, 34). These cells are then grouped into clones that have been derived from a common ancestor. While such techniques go to a more recent common ancestor compared to bulk sequencing, they are still not ideal as data derived from these approaches do not reflect mutational processes that are taking place in existing cells. Furthermore, reducing the depth of sequencing per cell to enable sequencing large numbers of cells, reduces the breadth of coverage, which is already compromised by loss of genetic materials during preparation steps. This increases the number of cells that are needed to be analysed for inferring and identifying clones which in turn moves the ancestor further back into the past. In addition, the lack of information about physical relatedness in single cell analysis methods, results in loss of an opportunity to group cells that are likely to be from a single clone. This increases the gap between the ancestor of an inferred clone and the present time, making it difficult to define processes that are active within currently existing cells in a tumour.

DigiPico has the distinct advantage of enabling the preservation of spatial information. Analysing spatially-related cells, preserves physical relatedness and enables the assumption that physically related cells belong to an individual clone (9). Defining distinct structures that may have arisen from a tissue resident stem cell has also been suggested to identify and analyse clones. For example, cells from a single small intestinal crypt or a single endometrial gland could be reasonable expected to come from a single tissue-resident stem cell (35, 36). Under these circumstances, each anatomical unit defines a clone that may or may not have clone specific mutations that can be related to a mutational driver. Furthermore, sequencing data from a clone can be computationally used to infer subclones and predict more recent events that may have arisen within a clone. This is akin to what bulk sequencing and analysis achieves but at the level of a single clone that is composed of a limited number of cells. Preserving spatial information is also particularly interesting because of the recent developments in enabling spatial transcriptomics technologies. It is conceivable that combining highly accurate DNA sequencing with spatial transcriptomics would allow the dissection of genetic and non-genetic heterogeneity in tissues. In short, current technologies, for the analysis of small clones yield large number of false positive results making it impossible to obtain direct accurate clone specific information on a genome scale without exhausting validation. Combing data from multiple clones, is a common solution but moves the ancestor further back into the past. We have previously used this approach for the analysis of small collection of tumour cells (tumour islets) (33). Because of the uncertainty associated with the mutation calls from individual islets, it was necessary to only call mutations that were shared between all tumour islets and effectively identify only truncal mutations. This was then followed by independent validation of some 700 mutations using targeted sequencing. While this still yielded important biological insights, we were unable to study islet-specific mutations. DigiPico is now enabling the study of such mutations.

Overall, here we showed how the DigiPico library preparation method allows for the accurate distinction of true from artefactual mutations, given a picogram quantity of starting DNA material. We believe that the ability to identify somatic mutations from limited numbers of cells obtained from clinical samples marks an important improvement over the existing methodologies.

### ACCESSION NUMBERS

All sequencing data used in this study are available on EGA (EGAD00001005118).

### REFERENCES

1. Turajlic, S., Sottoriva, A., Graham, T. and Swanton, C. (2019) Resolving genetic heterogeneity in cancer. Nat. Rev. Genet., 10.1038/s41576-019-0114-6.
6. Chen, L., Liu, P., Evans, T. C. J. and Ettwiller, L. M. (2017) DNA damage is a pervasive cause of sequencing errors, directly confounding variant identification. Science, 355, 752-756.
7. Costello, M., Pugh, T. J., Fennell, T. J., Stewart, C., Lichtenstein, L., Meldrim, J. C., Fostel, J. L., Friedrich, D. C., Perrin, D., Dionne, D., et al. (2013) Discovery and characterization of artifactual mutations in deep coverage targeted capture sequencing data due to oxidative DNA damage during sample preparation. Nucleic Acids Res., 41, e67.
9. Martincorena, I., Fowler, J. C., Wabik, A., Lawson, A. R. J., Abascal, F., Hall, M. W. J., Cagan, A., Murai, K., Mahbubani, K., Stratton, M. R., et al. (2018) Somatic mutant clones colonize the human esophagus with age. Science, 362, 911-917.
11. Dong, X., Zhang, L., Milholland, B., Lee, M., Maslov, A. Y., Wang, T. and Vijg, J. (2017) Accurate identification of single-nucleotide variants in whole-genome-amplified single cells. Nat. Methods, 14, 491-493.
12. Zafar, H., Wang, Y., Nakhleh, L., Navin, N. and Chen, K. (2016) Monovar: single-nucleotide variant detection in single cells. Nat. Methods, 13, 505-507.
14. Krueger F. (2016) Trim Galore!
15. Langmead, B. and Salzberg, S. L. (2012) Fast gapped-read alignment with Bowtie 2. Nat Meth, 9, 357-359.
17. Kim, S., Scheffler, K., Halpern, A. L., Bekritsky, M. A., Noh, E., Källberg, M., Chen, X., Kim, Y., Beyter, D., Krusche, P., et al. (2018) Strelka2: fast and accurate calling of germline and somatic variants. Nat. Methods, 15, 591-594.
18. Hosokawa, M., Nishikawa, Y., Kogawa, M. and Takeyama, H. (2017) Massively parallel whole genome amplification for single-cell sequencing using droplet microfluidics. Sci. Rep., 7, 5199.
19. Peters, B. A., Kermani, B. G., Sparks, A. B., Alferov, O., Hong, P., Alexeev, A., Jiang, Y., Dahl, F., Tang, Y. T., Haas, J., et al. (2012) Accurate whole-genome sequencing and haplotyping from 10 to 20 human cells. Nature, 487, 190-195.
20. Picard Tools (2018).
21. Rimmer, A., Phan, H., Mathieson, I., Iqbal, Z., Twigg, S. R. F., Wilkie, A. O. M., McVean, G. and Lunter, G. (2014) Integrating mapping-, assembly- and haplotype-based approaches for calling variants in clinical sequencing applications. Nat. Genet., 46, 912-918.
23. Danecek, P., Auton, A., Abecasis, G., Albers, C. A., Banks, E., DePristo, M. A., Handsaker, R. E., Lunter, G., Marth, G. T., Sherry, S. T., et al. (2011) The variant call format and VCFtools. Bioinformatics, 27, 2156-2158.
28. Arbeithuber, B., Makova, K. D. and Tiemann-Boege, I. (2016) Artifactual mutations resulting from DNA lesions limit detection levels in ultrasensitive sequencing applications. DNA Res., 23, 547-559.
29. Amini, S., Pushkarev, D., Christiansen, L., Kostem, E., Royce, T., Turk, C., Pignatelli, N., Adey, A., Kitzman, J. O., Vijayan, K., et al. (2014) Haplotype-resolved whole-genome sequencing by contiguity-preserving transposition and combinatorial indexing. Nat. Genet., 46, 1343-1349.
30. Zheng, G. X. Y., Lau, B. T., Schnall-Levin, M., Jarosz, M., Bell, J. M., Hindson, C. M., Kyriazopoulou-Panagiotopoulou, S., Masquelier, D. A., Merrill, L., Terry, J. M., et al. (2016) Haplotyping germline and cancer genomes with high-throughput linked-read sequencing. Nat. Biotechnol., 34, 303-311.
33. Hellner, K., Miranda, F., Fotso Chedom, D., Herrero-Gonzalez, S., Hayden, D. M., Tearle, R., Artibani, M., KaramiNejadRanjbar, M., Williams, R., Gaitskell, K., et al. (2016) Premalignant SOX2 overexpression in the fallopian tubes of ovarian cancer patients: Discovery and validation studies. EBioMedicine, 10, 137-149.
34. Laks, E., Zahn, H., Lai, D., McPherson, A., Steif, A., Brimhall, J., Biele, J., Wang, B., Masud, T., Grewal, D., et al. (2018) Resource: Scalable whole genome sequencing of 40,000 single cells identifies stochastic aneuploidies, genome replication states and clonal repertoires. bioRxiv, 10.1101/411058.
35. Moore, L., Leongamornlert, D., Coorens, T. H. H., Sanders, M. A., Ellis, P., Dawson, K., Maura, F., Nangalia, J., Tarpey, P. S., Brunner, S. F., et al. (2018) The mutational landscape of normal human endometrial epithelium. bioRxiv, 10.1101/505685.
36. Lee-Six, H., Ellis, P., Osborne, R. J., Sanders, M. A., Moore, L., Georgakopoulos, N., Torrente, F., Noorani, A., Goddard, M., Robinson, P., et al. (2018) The landscape of somatic mutation in normal colorectal epithelial cells. bioRxiv, 10.1101/416800.
37. Burgess, D. J. (2019) Spatial transcriptomics coming of age. Nat. Rev. Genet., 20, 317.

All references described herein may be incorporated by reference.

### DigiPico2

### EXAMPLE 2—DIGIPICO2, A NOVEL METHOD FOR WHOLE GENOME SEQUENCING OF PICOGRAM QUANTITIES OF DNA WITH UNPRECEDENTED ACCURACY

### Introduction

Previously we described DigiPico library preparation pipeline and MutLX analysis platform as a method for accurate identification of single nucleotide variants (SNVs) from limited amount of clinical material. This was an important methodological advancement mainly due to the fact that the limited amount of genetic material obtained from clinical samples must be whole genome amplified (WGA) prior to sequencing. The process of WGA however introduces up to 100,000 artefactual mutations in the amplified DNA which inundates the final analysis results with false positive variant calls that hamper any meaningful genetic interpretation from the original sample. In DigiPico strategy we overcome this obstacle by separating individual molecules of DNA into independent compartments before the WGA step and indexing them after the process. By doing so we digitize the information for real mutations, meaning each compartment will either carry the mutated allele or not. Artefactual mutations, however, because of the way that they are generated during the WGA process, will result in compartments that will contain both the mutated and the reference allele information (FIG. 1B). However, these artefacts can be removed with the use of machine learning methods.

While producing high quality data, DigiPico library preparation method, however, suffers from few technical limitations. Firstly the fragmentation step of the library preparation (CoREF), which was borrowed from a previously described method, is extremely complex and time consuming. Moreover, CoREF requires the use of dUTP during the WGA process. Since dUTP is an unnatural nucleotide it is likely that it may introduce further artefactual mutations in the final products. Next, we found that the adapter ligation efficiency was very low in DigiPico which could sometimes compromise the library quality. Lastly, because of the high number of indices and lack of redundancy in the index information there is a chance for index cross-contamination which could adversely affect the final results. Therefore, we developed DigiPico2 library preparation method to address all these issues.

### Results

### Improving the DigiPico Library Preparation Workflow

As explained previously, the use of dUTP in the DigiPico method is because of the requirement of CoREF fragmentation procedure, which is a very complex fragmentation strategy (FIG. 4A). Therefore using an alternative fragmentation method would solve the complexity and the dUTP issues at the same time. For this we decided to use an existing fragmentation and end-repair strategy offered by the Lotus DNA library preparation kit (IDT, USA). In Lotus DNA library preparation kit, a cocktail of enzymes is used to fragment the large DNA molecules into smaller pieces in a time dependent manner, and to prepare the termini of the fragments for the ligation step. To make this new strategy compatible with DigiPico we initially ensured that all compartments receive the enzyme cocktail at more or less the same time using the I-DOT (Dispendix, Germany) dispenser. Next we optimized the reaction conditions to achieve the desired fragment lengths for DigiPico sequencing. Doing so we were able to reduce the library preparation time to 4.5 hours from the original 12 hours and eliminate the need to use dUTP in the WGA reaction (FIG. 4B).

Next, we aimed at addressing the low ligation efficiency in DigiPico. Originally our adapter ligation and indexing relied on using an asymmetric ligation approach. In this approach long indexing oligos with short complementary regions were used for ligation which is extremely inefficient (FIG. 5A). A more efficient approach would require a looped common adapter that is ligated to both ends of the fragments. But because these adapters do not contain any indices in their standard form, the products need to be individually purified and then indices are introduced later through a PCR reaction using indexing primers. This however adds another challenge because purifying 384 independent products will be extremely complex, time consuming, and adds a significant risk of cross-contamination. To overcome these issues, we designed a novel indexing strategy for DigiPico2. In DigiPico2, one set of indices are first introduced in the stem loop of the common adapter (FIG. 5B). At the ligation step, all wells in each column of the plate will receive a different indexing looped common adapter, therefore a total of 24 different oligos would be sufficient to index all columns of the plate with the first set of indices (Column indices). After the ligation step, all wells in each row will be pooled into a separate tube, resulting in 16 different pools. These 16 different pools can conveniently be purified to be used for the next step of indexing. At the next step the purified products from each pool will be indexed through a PCR reaction using 16 different indexing primers (Row indices). At the end of this stage each well of the plate will have received a different column-row index combination. Using this indexing strategy not only will significantly improve the ligation efficiency but also introduces 2 sets of redundancy that can be employed to eliminate index cross-contamination from the data. In the first set, the column indices will bind to both end of each fragment. Therefore any cross contamination will most likely result in fragments that have unidentical indices at their termini and so can easily be removed from the data. In the second set, the indexing oligos for each row can be dually indexed so that both the standard index 1 (17) and standard index 2 (15) sequences can uniquely identify a specific row. Combining these sets of redundancy the index cross-contamination rate can be reduced by at least 2 orders of magnitude.

### DigiPico2 Workflow Significantly Improves the Library Quality

To test the effect of these modification on the final quality of the data, DigiPico2 sequencing was performed on 120 μg of DNA from blood of patient 11152. This sample was used because we had previously extensively sequenced the tumour and normal cells from this patient. As expected the WGA, similar to the previous version, resulted in a very uniform distribution of products (FIG. 6A). After library preparation and sequencing, however, it became clear that unlike with DigiPico, in DigiPico2 the representation of each well in the finally library appears to strongly correlate with the amount of WGA products (FIG. 6A-C). This is likely a direct result of an improved ligation efficiency. This improved correlation can also allow for introduction of a QC measure, solely based on the uniformity of the WGA products which previously was not possible. Moreover, analysing the index redundancy information we found that nearly 5% of the reads were eliminated using the index cross-contamination filter. Without this new filter these contaminants could have negatively affect the results of the analysis.

### DigiPico2 Protocol

200 μg of purified DNA, 20-30 resuspended nuclei, or laser-capture micro-dissected tumour islets, were first denatured using 5 μl of D2 buffer from Repli-g single cell kit (Qiagen). After 5 minutes incubation at room temperature, 95 μl of water was added to the sample and then using Mosquito HTS liquid handler (TTP Labtech) 200 nl of the denatured template was added to each well of a 384-well reaction plate already containing 800 nl of WGA mix (0.58 μl Sc Reaction Buffer, 0.04 μl Sc Polymerase (REPLI-g Single Cell kit, Qiagen), 0.04 μl EvaGreen 20× (Biotium), and 0.065 μl water). The plate was incubated at 30° C. for 1.5 hours followed by heat inactivation at 65° C. for 15 minutes. Addition of EvaGreen in the reaction allows for monitoring of the WGA reaction using a real-time PCR machine if required. Next, 250 nl of the WGA reactions was transferred to a new plate and 1.1 μl of NEBNext Ultra II FS Reaction mixture (753 nl water, 270 nl Ultra II FS Reaction Buffer, and 77 nl Ultra II FS Enzyme Mix) was added to each well using I-DOT dispenser (Dispendix). Plate was incubated at 37° C. for 6 minutes followed by incubation at 65° C. for 30 minutes. Next 150 nl of DigiPico indexed loop-adapters carrying column indices was added to all wells. Please note that all wells within the same column would receive the same indexing oligo at this stage. Next 1.2 μl of Ultra II Ligation mix (1150 nl Ultra II Ligation Master Mix, 38 nl Ligation Enhancer, and 12 nl water) was added to each well using Mosquito liquid handler with 5 cycles of mixing and the plate was incubated at 20° C. for 15 minutes followed by heat inactivation at 65° C. for 10 minutes. Next all wells within same row were pooled together using Mosquito liquid handler. Then 1.5 μl of USER enzyme (NEB) was added to 20 μl of pool from each row and the reaction was incubated at 37° C. for 15 minutes. USER enzyme cuts the looped adapters at the Uracil position. Next the products were size selected using SPRI beads to achieve a size range of 300-400 bp. Products from each row were then amplified using row indexing primers for 4 cycles. The final products were pooled together and the final library was purified using SPRI beads.

### Indexed Looped-Adapter—Column Indices

(some parts of the sequence from the instruction manual of library preparation (NEBNext® Multiplex Oligos for Illumin® (Index Primers Set 1))—https://international.neb.com/-/media/nebus/files/manuals/manuale7335.pdf?rev=4bf1622b342b4d73a2b01443068ed 2c5&hash=B049D91A18CDB471AB388DC6E67E06B79263E5C5)

P-[index′]
(SEQ ID NO: 1)
AGATCGGAAGAGCACACGTCTGAACTUCCCTACA
CGACGCTCTTCCGATCT[index]*T

Where P is a 5′ phosphate group and * shows a phosphorothioate bond. Index=a column index (Ci) or row index (Ri) sequence acting as a unique barcode for each column or row respectively.

Row indexing primers (oligonucleotide Sequences© 2007-2013 Illumina, Inc. All rights reserved.)

P5:
(SEQ ID: NO: 2)
AATGATACGGCGACCACCGAGATCTACAC[r-index]
ACACTCTTTCCCTACACGACGCTCTTCCGATC*T
P7:
(SEQ ID: NO: 3)
CAAGCAGAAGACGGCATACGAGAT[r-index]
GTGACTGGAGTTCAGACGTGTGCTCTTCCGATC*T
* shows a phosphorothioate bond.

### EXAMPLE 3—PICOCNV: ALLELE-SPECIFIC SOMATIC COPY NUMBER FROM PICOGRAMS OF DNA

### Summary

Copy number alterations are an important class of somatic alterations in cancer. Accurately identifying them is challenging, especially from microscopic samples. We present a method, PicoCNV, to call allele-specific copy number alterations from picogram quantities of DNA, corresponding to samples of just tens of cells. We extensively benchmark PicoCNV and employ it to inform the clinical management of a patient with minimal residual disease. Combined with our other recently-developed methods, PicoCNV allows for accurate and comprehensive genomic characterisations of cancers in settings where only microscopic samples are available.

### Introduction

Cancer is a genomic disease. The driving force behind each cancer is a repertoire of genomic changes known as somatic alterations (1, 2). Understanding how these alterations drive cancer is one of the central aims of cancer genomics (3), and efforts in this field have brought about clinical benefits including improved patient stratification, new prognostic biomarkers and an arsenal of new therapies (4, 5).

Copy number alterations (CNAs) are an important class of somatic alterations in cancer in which large segments of the genome are either amplified or deleted. They have been found to drive the cancer phenotype (6, 7), play a prominent role in cancer evolution (8), and hold substantial prognostic value (9, 10). They are particularly important in genomically unstable cancer types such as ovarian (11), oesophageal (12) and gastric cancers (13). Due to this importance, researchers have developed numerous algorithms to call somatic CNAs from sequencing data, including ASCAT (14), ABSOLUTE (15), Control-FREEC (16), OncoSNP-SEQ (17), and TITAN (18), among many others. In addition to determining the total number of copies at each locus in the genome, many of these algorithms also determine the relative numbers of the two parental alleles. Such methods are said to call allele-specific CNAs. This added allele-specific information allows researchers to identify subtle CNAs such as copy number neutral loss of heterozygosity (LOH) (14), which can have important clinical implications in cancer (19).

While early efforts tended to analyse bulk tumour samples, recent research has become more directed towards microscopic samples. For example, studies have investigated the therapeutic opportunities associated with circulating tumour cells (20), minimal residual disease (21, 22) and cancer sub-populations such as tumour initiating cells (23). One approach for characterising the genomes of such samples is single cell sequencing. Several CNA-calling algorithms have been developed that combine shallow whole genome sequencing (WGS) data across hundreds of cells to call CNAs at single-cell resolution. Established algorithms for calling total copy number include Ginkgo (24) and AneuFinder (25). More recently, researchers developed allele-specific single cell algorithms, including CHISEL (26) and Alleloscope (27). However, while they may provide accurate CNA calls, single cell methods are still limited in their accuracy to genotype single nucleotide variants (SNVs) (28), which are complementary to CNAs and can be highly consequential for therapeutics (29). An ideal platform for sequencing microscopic tumour samples would generate both accurate SNVs and CNAs for a complete genomic characterisation. Moreover, many single cell methods rely on aggregating data across hundreds or even thousands of cells, and are therefore unsuitable when total sample size is limited to tens of cells.

To investigate the genomes of tumour samples comprising tens of cells (e.g. obtained by laser capture microdissection), we recently developed a linked-read sequencing platform called DigiPico. DigiPico allows accurate identification of clonal SNVs and short insertions and deletions (indels) from picogram quantities of DNA (30). In the DigiPico protocol, we first distribute large fragments of genomic DNA, on the order of 100 kb in length, across one or more 384-well plates. Within each well separately, we then amplify, fragment and barcode the material. Performing the amplification step independently in each well allows us to computationally identify and remove artefactual mutations arising from oxidation and spontaneous deamination. Intuitively, real mutations have support from a larger proportion of wells than artefactual mutations, which tend to appear in only a single well due to the random nature of the DNA damage. We developed a machine learning algorithm, MutLX, to exploit this effect to reliably identify the real mutations in a DigiPico sample (30).

Given the biological and prognostic importance of somatic CNAs, we sought to develop a method to accurately call somatic allele-specific CNAs from DigiPico sequencing data. We noted that existing CNA calling methods for linked-reads only produced total copy number, and were not allele-specific (31). However, there were already numerous established methods for calling allele-specific CNAs from bulk tumour data. Therefore, we aimed to adapt one such pipeline to DigiPico's linked-read data by exploiting DigiPico's characteristic structure of independent wells, rather than to develop an entirely new algorithm. We chose to adapt the TITAN algorithm (18) due to its modular design, and termed the resulting method PicoCNV. To develop this method, we used our available DigiPico samples taken from ovarian cancers (Table S1), which was a suitable cancer type due to its high level of chromosomal instability (11).

Table S1: Ovarian cancer samples used in study. The optimisation set was used to systematically optimise and benchmark the PicoCNV pipeline. The MRD set was then used to demonstrate clinical practicability. Internal patient and sample IDs are in brackets.

| | Normal | Bulk | | DigiPico | | | | |
|---|---|---|---|---|---|---|---|---|
| | WGS | WGS | DigiPico | DNA | DigiPico | | | |
| Patient | sample | sample | sample | input | wells | Sample | Tumour | |
| ID | ID | ID | ID | (pg) | (n) | set | histology | Notes |
| A | A-N1 | A-B1 | A-D1 | 240 | 384 | Optimisation | HGSOC | |
| (11152) | | (1101) | (1111) | | | | | |
| | | | A-D2 | 360 | 1152 | | | |
| | | | (1211- | | | | | |
| | | | 1213) | | | | | |
| | | A-B2 | | | | | | Low-purity |
| | | (1201) | | | | | | bulk sample |
| | | | | | | | | (estimated |
| | | | | | | | | 45%) |
| B | B-N1 | B-B1 | B-D1 | 560 | 1152 | | Ovarian | ASCAT failed |
| (11611) | | (SB- | (SB- | | | | carcinosarcoma | to solve |
| | | 11611- | 11611- | | | | | purity/ploidy |
| | | BG) | TD) | | | | | for bulk or |
| | | | | | | | | DigiPico |
| C | C-N1 | — | C-D1 | 1620 | 1152 | MRD | High grade | |
| (11617) | | | (SB- | | | | serous | |
| | | | 11617- | | | | fallopian | |
| | | | TD) | | | | tube | |
| | | | | | | | carcinoma | |

### Results

Refining copy number signals in DigiPico sequencing data

We first investigated the quality of the raw DigiPico data. To derive allele-specific copy number from sequencing data, most algorithms require two numeric quantities along the genome: the log ratio (logR); and the B-allele frequency (BAF). The logR is the logarithm of the ratio of read counts in the tumour sample to the normal sample, and it describes the total copy number. It can be calculated at individual points in the genome (14) or in bins to make the signal more robust to noise (18, 25). The BAF instead encapsulates the allelic ratio of parental alleles, and is the ratio of reads supporting the reference allele versus the alternative allele at heterozygous SNPs.

We initially explored the data for the matched bulk-DigiPico sample pair A-B1/A-D1 (Table S1), and found that DigiPico sequencing data was sufficient to derive both logR and BAF values. However, compared to standard bulk sequencing data from the same tumour, the raw DigiPico-derived logR and BAF both exhibited very high levels of noise (FIG. 7A, B). This likely reflected the statistical noise introduced by sequencing picogram quantities of DNA. We therefore reasoned that de-noising the logR and BAF data could increase the accuracy of CNA calling from DigiPico data.

Many CNA calling methods, including TITAN, measure the logR in bins along the genome (18, 25). When we applied this approach to DigiPico data we found that the noise level, as measured by the variance of the logR for distinct copy number states, was over 150 times greater than for bulk data (FIG. 8). However, by increasing the bin size from TITAN's default of 10 kb to 100 kb, we reduced the logR variance by a factor of 3.1. We achieved a further reduction of the logR variance by exploiting the independence of wells in DigiPico. In DigiPico, we amplify and fragment large molecules of DNA separately in hundreds of wells. The efficacy of amplification varies randomly from well to well and molecule to molecule, resulting in considerable noise in read count data. To counteract this, we replaced the count of short sequencing reads from the DigiPico data with a count of reconstructed large fragments (RLFs, Methods). By using a logR derived from RLF counts, we reduced the logR variance by a further factor of 2 (FIG. 8). Therefore, by increasing the bin size and deriving the logR from RLFs instead of short reads, we were able to considerably reduce the noise present in the logR data. However, we noted that the de-noised logR data was still of lower quality than that obtained from bulk sequencing.

We next sought to clean up the BAF signal in a similar way to the logR. Again exploiting the independence of wells in DigiPico, we calculated the BAF from counts of wells rather than counts of short reads. To do this, for each heterozygous SNP we first identified the covering wells as those containing at least one read aligned to that SNP's position. For SNPs with at least five covering wells, we then counted the number of supporting wells as how many wells contained at least 80% variant reads. Finally, we divided the number of supporting wells by the number of covering wells to give the well count-derived BAF. Similar to counting RLFs for the logR value, counting wells instead of reads was a more robust approach to the noise introduced by well-to-well variances in DNA amplification. By using well counts, we were able to produce a cleaner BAF signal from DigiPico data (FIG. 9A-C). We achieved a further improvement by using the well-based structure of DigiPico to aggregate SNPs into phase groups (Methods). Since nearby SNPs in the same phase group (i.e. on the same parental allele) had the same underlying allele frequency (32), averaging over groups of SNPs reduced the noise from SNP-to-SNP variation (26, 27) (FIG. 9D). Combined, these changes resulted in a remarkably clean BAF signal that was almost indistinguishable from bulk sequencing data.

Having de-noised both the logR and BAF signals from DigiPico data (FIG. 7C), we applied the TITAN algorithm (18) to call allele-specific somatic CNAs. However, we found that CNA calls from the cleaned DigiPico data with default TITAN parameters were unrealistically fragmented, indicating that the algorithm had fit its model to noise rather than genuine signal (FIG. 7D). Indeed, CNA segments shorter than 1 Mb in length covered 62% of the genome for the de-noised DigiPico data, compared to just 6% for matched bulk data (FIG. 7E). This observation prompted us to modify TITAN's expected segment length parameter. We reasoned that by increasing this parameter, we would be able to force the algorithm to fit to the underlying signal more robustly.

### Systematic Pipeline Optimisation

We had so far motivated changing five settings to adapt the TITAN CNA calling pipeline for DigiPico data: increasing the logR bin size: deriving the logR from RLF counts instead of read counts: deriving the BAF from well counts instead of read counts; phase group-aggregating BAF values; and increasing TITAN's segment length parameter. While each individual adaptation was justified, we wanted to ensure that the overall combination produced optimal CNA calls. To achieve this, we applied a three-step optimisation approach to three ovarian cancer DigiPico samples for which matched bulk sequencing data were available (optimisation set, Table S1). In our approach we considered TITAN CNA calls from bulk data to be ground truth. First, in a grid search over the five settings, we ran 240 different pipelines covering all combinations listed in Table S2. Second, for each implementation we measured how closely the CNA calls from DigiPico data followed TITAN CNA calls from matched bulk data using two root mean squared error (RMSE) metrics. These RMSE metrics considered accuracy in terms of both total copy number and allelic ratio (Methods). Third, we combined the RMSE values across these two metrics and across the optimisation sample set to produce a single RMSE z-score for each of the 240 pipelines (Methods). We selected the final optimised PicoCNV pipeline as the one with the smallest RMSE z-score.

Table S2: Systematic optimisation of PicoCNV pipeline. For each of the five settings, the values explored are listed. Every combination was considered, for a total of 240 pipeline implementations.

| | Values | |
|---|---|---|
| Setting | (n) | Values |
| LogR bin size | 3 | 10 kb, 100 kb, 1 Mb |
| LogR count type | 2 | Reads, RLFs |
| BAF count type | 2 | Reads, wells |
| BAF aggregation | 2 | SNPs, phase groups |
| TITAN segment length | 10 | 10¹⁵, 10¹⁶, 10¹⁷, 10¹⁸, 10¹⁹, 10²⁰, 10²¹, |
| parameter | | 10²², 10²³, 10²⁴ |

As expected, the best-performing pipeline included all five of the above proposed adaptations to DigiPico data. Interestingly, the marginal RMSE distributions did not support every setting change, indicating the importance of a fully combinatorial grid search approach (FIG. 10). To contextualise the RMSE values, we analysed a secondary low-purity bulk sample from patient A (sample A-B2, Table S1, TITAN-estimated purity 45%) to use as a benchmark. We ran the standard TITAN pipeline on this sample and measured its RMSEs relative to the primary bulk sample A-B1. Remarkably, many of the DigiPico CNA call sets reflected the primary bulk results more accurately than the low-purity bulk sample (FIG. 10). The final PicoCNV CNA calls bore a clear resemblance to their matched bulk profiles (FIG. 11), indicating that PicoCNV was able to accurately call somatic CNAs from DigiPico sequencing data.

### Comparison to Other Algorithms

Most of the adaptations we had made to the standard TITAN workflow amounted to data de-noising, and could therefore be applied to almost any CNA calling method for use with DigiPico data. To assess TITAN's suitability as our algorithm of choice, we sought to measure its performance relative to ASCAT (14), another well-established allele-specific bulk CNA calling algorithm. To that end, we established a DigiPico-adapted ASCAT pipeline that made use of many of the same adaptations we had implemented to create PicoCNV and applied it to our optimisation sample set (Methods).

In the absence of genuine ground truth data, we sought to measure the robustness of both CNA calling algorithms to the noise inherent in DigiPico data. We reasoned that the desired behaviour of an algorithm was generating CNA calls from DigiPico data that resembled the CNA calls from matched bulk data as closely as possible. As before, we measured the similarity between the bulk and DigiPico CNA calls using the RMSEs of total copy number and allelic ratio (Methods), with smaller RMSE values representing greater similarity. As expected, we found that the DigiPico-adapted pipelines generally performed better than the default pipelines for both TITAN and ASCAT (FIG. 12A, B). We also found that, when adapted to DigiPico data, TITAN outperformed ASCAT with median RMSE values of 1.0 and 0.13 compared to 2.2 and 0.18 for total copy number and allelic ratio respectively. Finally, we noted that ASCAT was unable to fit purity or ploidy values for patient B, requiring manual estimation of both. We were therefore confident that TITAN was an appropriate choice of CNA calling algorithm for our pipeline.

Given that DigiPico and single cell WGS face some similar challenges, we were also interested to see how PicoCNV performed relative to an established single cell CNA calling method. As a comparison, we ran AneuFinder (25) on both raw and de-noised DigiPico data (Methods). Since AneuFinder does not determine allele-specific copy number, we then measured only the total copy number RMSE between AneuFinder's CNA calls and the TITAN CNA calls from matched bulk data. We found that PicoCNV out-performed AneuFinder, with a median RMSE of 1.0 compared to 1.6 and 1.7 for AneuFinder using raw and de-noised DigiPico data, respectively (FIG. 12C). Interestingly, AneuFinder performed marginally worse overall with de-noised DigiPico data than with raw DigiPico data. This may have reflected the fact that AneuFinder was already optimised for use with noisy single cell sequencing data, similar to the raw DigiPico output. Overall, we concluded that PicoCNV performed well at calling allele-specific somatic CNAs from DigiPico data compared to other algorithms.

### PicoCNV Application to MRD

Having established PicoCNV's capability to accurately call somatic CNAs from picogram quantities of DNA, we next sought to apply it in a setting where standard bulk sequencing was not possible. Minimal residual disease (MRD) in solid tumours refers to the microscopic deposits of cancer cells remaining after a patient has responded well to first-line treatments, often debulking surgery and chemotherapy. MRD is the ultimate source of relapse, so being able to molecularly characterise it could enable therapeutics to target it specifically and eventually improve patient outcomes. We applied DigiPico sequencing and PicoCNV to a previously un-sequenced high grade serous fallopian tube carcinoma MRD sample (C-D1). PicoCNV produced visually clean tracks of logR, BAF and CNA calls along the genome (FIG. 13A), indicating that data de-noising was effective in this sample. The CNA calls showed evidence of a whole genome doubling event (fitted ploidy value 3.8), which is a common event occurring in approximately 40% of ovarian tumours (33).

Since sample C-D1 was taken by laser capture microdissection at a time when the patient had no macroscopic disease, no bulk sample was available to quantitatively validate the PicoCNV CNA calls. In light of this, we used two orthogonal validation approaches to ensure that the CNA calls were trustworthy. First, we reasoned that total copy number should be positively correlated with gene expression. To verify this, we used matched RNA-Seq data to quantify gene expression (Methods), and compared expression levels with copy number states. We found, as expected, that genes with copy number losses had overall lower expression, while genes with copy number gains had higher expression (FIG. 13B). Moreover, a linear model strongly supported the statistical significance of this positive correlation (p=1.3×10⁻⁴²). As a second orthogonal validation, we reasoned that in general oncogenes (OGs) should be amplified, while tumour suppressor genes (TSGs) should be deleted. To verify this, we first obtained a list of 17 canonical driver genes in ovarian cancer (34), each of which is known to be either an OG or a TSG. We found that when they were affected by CNAs, OGs tended to be amplified (100%, two out of two), while TSGs were more likely to be deleted (67%, four out of six) (FIG. 13C). TP53 was the only canonical driver gene with a copy number as low as two, and we also found evidence that it underwent loss of heterozygosity (LOH, FIG. 14A). TP53 LOH is known to be very common in ovarian cancer (75%) (35). Overall, we concluded that our orthogonal validations supported the validity of PicoCNV's CNA calls in this MRD sample.

Finally, we sought to establish the clinical utility of PicoCNV in the MRD setting. Maintenance therapy has recently become a viable option to delay relapse in ovarian cancer patients who respond well to first-line therapy (36). We used PicoCNV to identify clinically relevant CNAs that might inform how the patient's maintenance therapy would be managed. Amplifications of CCNE1 are a biomarker of poor prognosis, but various therapies have also been proposed specifically for CCNE1-amplified ovarian cancers, including WEE1 kinase and CDK2 inhibitors (37), and proteasome inhibitors (38). However, in our MRD sample we found clear evidence of a lack of CCNE1 amplification, suggesting that such approaches would not be appropriate (FIG. 14B). A recent clinical trial (ARIEL3) (39) found that homologous recombination deficiency (HRD) is predicative of ovarian cancer response to PARP inhibitors for maintenance therapy. The authors measured HRD using a combination of BRCA mutation status and the extent of genomic LOH. We found that sample C-D1 was wild type for both BRCA1 and BRCA2. We then used the PicoCNV CNA calls to measure the extent of LOH across the genome, and found that it was 22.9% (FIG. 13A). This would qualify as LOH-high in the ARIEL3 trial (threshold of 14%), which suggested that our patient would respond well to PARP inhibitors for maintenance therapy. We concluded that PicoCNV was able to provide clinically useful insights in microscopic settings where standard genomic sequencing would be unable to produce useful results.

### Discussion

We have presented the development and benchmarking of a new method, PicoCNV, for calling allele-specific CNAs from picogram quantities of DNA. In doing so, we have exploited the structure of independent wells in the DigiPico linked-read sequencing method. We adapted the TITAN (18) pipeline for calling CNAs from DigiPico data in five distinct ways. To ensure the quality of our final pipeline, we employed a grid search approach to systematically optimise PicoCNV. Further, we demonstrated that TITAN was a suitable choice of CNA calling algorithm for adaptation to DigiPico by benchmarking it against other similar algorithms. Finally, we demonstrated the clinical utility of PicoCNV by showing how it could be used to inform maintenance therapy in the MRD setting.

Most of the adaptations we made to the standard TITAN workflow for bulk cancer samples amounted to data de-noising, and could therefore be applied to almost any CNA calling algorithm. This demonstrates the utility of the underlying DigiPico sequencing method. Data from picogram quantities of DNA are inherently noisy, but performing amplification independently in hundreds of wells makes DigiPico robust to the well-to-well variation in amplification efficacy (30). Moreover, it allows nearby heterozygous SNPs to be accurately phased, which additionally reduces SNP-to-SNP noise. Thus, the structure of DigiPico was critical to our approach of de-noising the sequencing data. This is unsurprising since linked-read technologies were initially developed specifically to investigate structural genomic variations (31). We also note that, compared to the now-discontinued 10× linked read technology, DigiPico has the advantage of being able to work with less than Ing of starting DNA material.

We believe that PicoCNV is sufficiently accurate to enable us to accurately call allele-specific somatic CNAs from DigiPico data. In combination then, DigiPico, MutLX and PicoCNV will enable us to produce comprehensive and accurate genomic characterisations of samples comprising tens of cancer cells. Future studies could use this platform to study selected tumour cell sub-populations, minimal residual disease (MRD) and circulating tumour cells. They could also explore potential applications in liquid biopsies. We could additionally use our platform to examine how acquired resistance to therapies evolves at the MRD stage. In ovarian cancer in particular, where relapse rates after standard of care chemotherapy are very high (70-80%) (44), this remains a pressing question.

### Code Availability

PicoCNV code is available from https://process.innovation.ox.ac.uk/software, under an academic-use license.

### Methods

### Patient Samples

Written consent from study participants was obtained for participation in the prospective biomarker validation study Gynaecological Oncology Targeted Therapy Study 01 (GO-Target-01) under research ethics approval number 11/SC/0014. Tumour and blood samples were obtained on the day of surgery. Blood samples were processed to isolate peripheral blood mononuclear cells (PBMCs) using Lymphoprep™ (STEMCELL Technologies, Canada). Tumour tissue was dissociated using human Tumor Dissociation Kits (Miltenyi Biotec, Germany) following the manufacturer's protocol. Dissociated tumour cells were processed for staining and fluorescent activated cell sorting to obtain bulk cancer cells and tumour initiating cells (TICs) as described in Okamoto et al. (45).

### DNA and RNA Extraction and Library Preparation

DNA from PBMCs and bulk cancer cells was isolated using DNeasy blood and tissue kit (Qiagen, USA). The libraries for both germline and tumour DNA were constructed and sequenced by Novogene Co. Ltd (China).

RNA from bulk cancer cells was isolated using RNeasy Micro kit (Qiagen USA) following the manufacturer's instructions. Total RNA was eluted in nuclease free water and stored at −80° C. RNA-Seq libraries were prepared using the SMARTer Stranded Total RNA-Seq Kit v2 Pico Input (Takara Bio USA). The libraries were then sequenced using 75 bp paired-end reads on NextSeq500 platform.

### DigiPico Library Prep and Sequencing

For DigiPico sequencing, DNA was isolated from TICs using Repli-g single cell kits (Qiagen, USA). DigiPico library prep was performed as described in Carrami et al. (30). The libraries were then sent to a sequencing company (Novogene Co. Ltd, China) and were sequenced on a Novaseq platform in 150 bp paired-end sequencing.

### Data Pre-Processing

Bulk tumour and normal blood-derived whole genome sequencing data were trimmed of Illumina adapters with Trim Galore v0.6.6 (46) and aligned to the human genome version hg19 using Bwa-mem v2.2.1 (47). PCR duplicate reads were removed from the resulting BAM files using Picard Tools v2.18.17 (48). Heterozygous SNPs were obtained for each patient by genotyping common SNP loci from HapMap v3.3 (49) using samtools mpileup and bcftools call v1.14 (50), and sub-setting the resulting VCFs to retain only SNPs with the 0/1 genotype.

For DigiPico data, raw paired-end whole genome sequencing data were demultiplexed to give two FASTQ files per well using custom scripts. Reads from each well were then trimmed of Illumina adapters using Trim Galore v0.6.6 (46) and aligned to the human genome version hg19 using Bowtie v2.4.1 (51). The resulting BAM files were cleaned of PCR duplicates with Picard Tools v2.18.17 (48).

Construction of RLFs from DigiPico Data

The original large DNA fragments distributed into DigiPico wells were reconstructed in silico to give reconstructed large fragments (RLFs). Separately for each chromosome in each well, read pairs were determined to come from the same RLF if there was a gap between them shorter than 10 kb in length. This approach assumed that the original large DNA fragments were non-overlapping. While this was not strictly enforced by the DigiPico protocol, it was estimated to be true in 92% of cases based on counts of biallelic wells at heterozygous SNPs in sample A-D1. Thus, the RLF reconstruction approach was deemed to be broadly accurate.

SNP Phasing from DigiPico Data

The well-based structure of DigiPico allowed heterozygous SNPs to be assigned locally to phase groups. This relied on the fact that nearby SNPs from the same allele would tend to co-localise on the same large DNA fragments, and thus would be supported by the same wells. A heuristic approach was used to exploit this effect.

First, SNPs on each chromosome were subsetted for those covered by at least five wells. For each SNP, the nearest neighbouring SNPs within 10 kb were considered, up to a maximum of five neighbours. The well concordance of each pair was calculated as the percentage of wells covering both SNPs that agreed on its status, i.e. that either supported both SNPs or supported neither SNP. A well was determined to support a SNP if it contained >80% variant reads. If 80% of wells agreed, the SNP pair was determined to be in phase (same allele). Conversely, if 80% of wells disagreed the pair was determined to be in anti-phase (opposite allele). In cases where results for overlapping sets of pairs were contradictory, a majority voting system was used to determine the most likely phase grouping.

### TITAN Implementations and PicoCNV Systematic Optimisation

TITAN (18) v1.32 was obtained from Bioconda (52). Bulk data were processed using the pipeline available at https://github.com/gavinha/TitanCNA/tree/master/scripts/snakemake. As part of this pipeline, logR data were corrected for GC content and mappability biases. Only fully clonal solutions were considered (numClusters command line argument set to 1), and the Markov model's starting ploidy value (ploidy_0 command line argument) was fixed at 2. Otherwise, default parameters were used except for bulk sample B-B1. In this sample, low-quality data resulted in unrealistically short CNA segments. To correct for this, we increased TITAN's segment length parameter (txnExpLen command line argument) from 10¹⁵ to 10²¹ for this sample only.

The PicoCNV pipeline was adapted from the same pipeline used for the bulk TITAN implementation. To systematically optimise PicoCNV, 240 pipelines were assessed covering all possible combinations of the settings listed in Table S2. The final optimised PicoCNV pipeline was adapted to DigiPico data in the following five ways: the logR bin size was increased from 10 kb to 100 kb: the logR was derived from counts of RLFs rather than counts of short reads: the BAF was calculated from well counts rather than read counts: the BAF was averaged over phased groups of heterozygous SNPs; and TITAN's expected segment length parameter (txnExpLen command line argument) was increased from 10¹⁵ to 10²¹.

DigiPico Vs Bulk RMSEs and z-Scores

CNA calls from DigiPico data were compared to CNA calls from bulk data, treating the bulk calls as ground truth. In the systematic optimisation of the PicoCNV pipeline, both bulk and DigiPico CNA calls were generated using the TITAN algorithm. When comparing to other algorithms, where possible the same algorithm was used for generating bulk and DigiPico CNA calls. Therefore, TITAN calls from DigiPico were compared to TITAN calls from bulk, and ASCAT DigiPico calls were compared to ASCAT calls from bulk. Since AneuFinder was designed for single cell data, AneuFinder DigiPico calls were instead compared to TITAN bulk calls.

The concordance between a set of DigiPico CNA calls and a ground truth set of bulk CNA calls was measured using two root mean squared error (RMSE) metrics: total copy number and allelic ratio. The smaller these RMSE values were, the more similar the DigiPico calls were determined to be to the corresponding ground truth bulk calls. The total copy number RMSE was calculated as

$${RMSE}_{n} = \sqrt{\frac{1}{\vert G \vert} {\sum }_{i ∈ G} {( {n}_{i}^{D} - {n}_{i}^{B} )}^{2}} , where {n}_{i}^{D} and {n}_{i}^{B}$$

are the total copy numbers at position i in the DigiPico and bulk calls respectively, and G is the set of all positions in the genome. Similarly, the allelic ratio RMSE was calculated as

$$R M S {E}_{r} = \sqrt{\frac{1}{\vert G \vert} {\sum }_{i ∈ G} {( {r}_{i}^{D} - {r}_{i}^{B} )}^{2}} , where {r}_{i}^{D} and {r}_{i}^{B}$$

are the allelic ratios at position i in the DigiPico calls and bulk calls respectively. The allelic ratios were calculated at each position as

$${r}_{i} = \frac{{b}_{i}}{{n}_{i}} ,$$

where bᵢ is the minor copy number and nᵢ is the total copy number.

In the systematic optimisation of the PicoCNV pipeline, RMSE values were combined to give a single score for each of the 240 implementations. First, for each pipeline the mean of both RMSE metrics was taken over the optimisation set of DigiPico samples. Next, the averaged RMSEs were converted to z-scores for the two metrics separately, by subtracting the mean and dividing by the standard deviation. This gave two intermediate RMSE z-scores per pipeline, one for each of the two metrics. Finally, the mean of the two z-scores was taken as the overall RMSE z-score for each pipeline. During the optimisation, the pipeline with the smallest overall RMSE z-score was taken as the optimal PicoCNV implementation.

### ASCAT Implementations

ASCAT (14) v2.5.2 was obtained from Bioconda (52). The ‘Standard ASCAT run’ pipeline from https://github.com/VanLoo-lab/ascat/tree/master/ExampleData, including GC correction, was used to process bulk and DigiPico data. The pipeline was adapted for DigiPico data in the following three ways: the logR was derived from counts of wells covering each heterozygous SNP rather than counts of short reads: the BAF was calculated from well counts instead of read counts; and the BAF was averaged over phased groups of SNPs. ASCAT failed to identify a unique purity and ploidy solution for either the bulk or DigiPico samples from patient B (Table S1). To address this, we manually fixed the ploidy at the value determined by TITAN from this patient's bulk sample (ploidy=3.8), and we fixed sample purities at values determined by manual inspection of BAF values in a LOH region for both the bulk.

### AneuFinder Implementations

AneuFinder (25) v1.22 was obtained from Bioconda (52). Due to the much larger size of DigiPico BAM files compared to the shallow single cell BAM files that AneuFinder was designed for (30-90×WGS compared to 1-2×WGS), we implemented the initial read counting step in a custom Python script. The pipeline was adapted to use de-noised DigiPico data in only one respect, namely that the logR was derived from counts of RLFs rather than short reads. The default logR bin size of 1 Mb was used in all cases. A blacklist of regions with low mappability was obtained from UCSC at http://hgdownload.cse.ucsc.edu/goldenpath/hg19/encodeDCC/wgEncode Mapability/w gEncodeDacMapabilityConsensusExcludable.bed.gz, and AneuFinder's GC content correction was applied. Since we only had a single library per tumour, we did not make use of AneuFinder's library clustering quality control tools.

### MRD RNA-Seq and Expression Quantification

RNA reads were trimmed of adapters using Trim Galore v0.6.6 (46), and transcript expression was quantified in TPM units using Kallisto v0.46.2 (53). Transcripts were then aggregated to genes using a list obtained from Ensembl release 103 (54).

### REFERENCES

1. Bailey M H, Tokheim C, Porta-Pardo E, Sengupta S, Bertrand D, Weerasinghe A, et al. Comprehensive Characterization of Cancer Driver Genes and Mutations. Cell. 2018; 173(2):371-85 e18.
2. Consortium ITP-CAoWG. Pan-cancer analysis of whole genomes. Nature. 2020; 578(7793):82-93.
3. Vogelstein B, Papadopoulos N, Velculescu V E, Zhou S, Diaz L A, Jr., Kinzler K W. Cancer genome landscapes. Science. 2013; 339(6127):1546-58.
4 Berger M F, Mardis E R. The emerging clinical relevance of genomics in cancer medicine. Nat Rev Clin Oncol. 2018; 15(6):353-65.
5. Malone E R, Oliva M, Sabatini P J B, Stockley T L, Siu L L. Molecular profiling for precision cancer therapies. Genome Med. 2020; 12(1):8.
6. Sondka Z, Bamford S, Cole C G, Ward S A, Dunham I, Forbes S A. The COSMIC Cancer Gene Census: describing genetic dysfunction across all human cancers. Nat Rev Cancer. 2018; 18(11):696-705.
7. Wang G, Anastassiou D. Pan-cancer driver copy number alterations identified by joint expression/CNA data analysis. Sci Rep. 2020; 10(1):17199.
8. Gerstung M, Jolly C, Leshchiner I, Dentro S C, Gonzalez S, Rosebrock D, et al. The evolutionary history of 2,658 cancers. Nature. 2020; 578(7793): 122-8.
9. Hieronymus H, Murali R, Tin A, Yadav K, Abida W, Moller H, et al. Tumor copy number alteration burden is a pan-cancer prognostic factor associated with recurrence and death. Elife. 2018; 7.
10. Smith J C, Sheltzer J M. Systematic identification of mutations and copy number alterations associated with cancer patient prognosis. Elife. 2018; 7.
11. Penner-Goeke S, Lichtensztejn Z, Neufeld M, Ali J L, Altman A D, Nachtigal M W, et al. The temporal dynamics of chromosome instability in ovarian cancer cell lines and primary patient samples. PLoS Genet. 2017; 13(4):e1006707.
12. Paulson T G, Maley C C, Li X, Li H, Sanchez C A, Chao D L, et al. Chromosomal instability and copy number alterations in Barrett's esophagus and esophageal adenocarcinoma. Clin Cancer Res. 2009; 15(10):3305-14.
13. Maleki S S, Rocken C. Chromosomal Instability in Gastric Cancer Biology. Neoplasia. 2017; 19(5):412-20.
14. Van Loo P, Nordgard S H, Lingjaerde O C, Russnes H G, Rye I H, Sun W, et al. Allele-specific copy number analysis of tumors. Proc Natl Acad Sci USA. 2010; 107(39):16910-5.
15. Carter S L, Cibulskis K, Helman E, McKenna A, Shen H, Zack T, et al. Absolute quantification of somatic DNA alterations in human cancer. Nat Biotechnol. 2012; 30(5):413-21.
16. Boeva V, Popova T, Bleakley K, Chiche P, Cappo J, Schleiermacher G, et al. Control-FREEC: a tool for assessing copy number and allelic content using next-generation sequencing data. Bioinformatics. 2012; 28(3):423-5.
17. Yau C. OncoSNP-SEQ: a statistical approach for the identification of somatic copy number alterations from next-generation sequencing of cancer genomes. Bioinformatics. 2013; 29(19):2482-4.
18. Ha G, Roth A, Khattra J, Ho J, Yap D, Prentice L M, et al. TITAN: inference of copy number architectures in clonal cell populations from tumor whole-genome sequence data. Genome Res. 2014; 24(11):1881-93.
19. Ryland G L, Doyle M A, Goode D, Boyle S E, Choong D Y, Rowley S M, et al. Loss of heterozygosity: what is it good for? BMC Med Genomics. 2015; 8:45.
20. Lin D, Shen L, Luo M, Zhang K, Li J, Yang Q, et al. Circulating tumor cells: biology and clinical significance. Signal Transduct Target Ther. 2021; 6(1):404.
21. Artibani M, Masuda K, Hu Z, Rauher P C, Mallett G, Wietek N, et al. Adipocyte-like signature in ovarian cancer minimal residual disease identifies metabolic vulnerabilities of tumor-initiating cells. JCI Insight. 2021; 6(11).
22. Luskin M R, Murakami M A, Manalis S R, Weinstock D M. Targeting minimal residual disease: a path to cure? Nat Rev Cancer. 2018; 18(4):255-63.
23. Qureshi-Baig K, Ullmann P, Haan S, Letellier E. Tumor-Initiating Cells: a criTICal review of isolation approaches and new challenges in targeting strategies. Mol Cancer. 2017; 16(1):40.
24. Garvin T, Aboukhalil R, Kendall J, Baslan T, Atwal G S, Hicks J, et al. Interactive analysis and assessment of single-cell copy-number variations. Nat Methods. 2015: 12 (11): 1058-60.
25. Bakker B, Taudt A, Belderbos M E, Porubsky D, Spierings D C, de Jong T V, et al. Single-cell sequencing reveals karyotype heterogeneity in murine and human malignancies. Genome Biol. 2016; 17(1):115.
26. Zaccaria S, Raphael B J. Characterizing allele- and haplotype-specific copy numbers in single cells with CHISEL. Nat Biotechnol. 2021; 39(2):207-14.
27. Wu C Y, Lau B T, Kim H S, Sathe A, Grimes S M, Ji H P, et al. Integrative single-cell analysis of allele-specific copy number alterations and chromatin accessibility in cancer. Nat Biotechnol. 2021; 39(10):1259-69.
28. Dong X, Zhang L, Milholland B, Lee M, Maslov A Y, Wang T, et al. Accurate identification of single-nucleotide variants in whole-genome-amplified single cells. Nat Methods. 2017; 14(5):491-3.
29. Morotti M, Albukhari A, Alsaadi A, Artibani M, Brenton J D, Curbishley S M, et al. Promises and challenges of adoptive T-cell therapies for solid tumours. Br J Cancer. 2021; 124(11):1759-76.
30. KaramiNejadRanjbar M, Sharifzadeh S, Wietek N C, Artibani M, El-Sahhar S, Sauka-Spengler T, et al. A highly accurate platform for clone-specific mutation discovery enables the study of active mutational processes. Elife. 2020; 9.
31. Marks P, Garcia S, Barrio A M, Belhocine K, Bernate J, Bharadwaj R, et al. Resolving the full spectrum of human genome variation using Linked-Reads. Genome Res. 2019; 29(4):635-45.
32. Vattathil S, Scheet P. Haplotype-based profiling of subtle allelic imbalance with SNP arrays. Genome Res. 2013; 23(1):152-8.
33. Bielski C M, Zehir A, Penson A V, Donoghue M T A, Chatila W, Armenia J, et al. Genome doubling shapes the evolution and prognosis of advanced cancers. Nat Genet. 2018; 50(8):1189-95.
34. Repana D, Nulsen J, Dressler L, Bortolomeazzi M, Venkata S K, Tourna A, et al. The Network of Cancer Genes (NCG): a comprehensive catalogue of known and candidate cancer genes from cancer sequencing screens. Genome Biol. 2019; 20(1):1.
35. Alexandrova E M, Mirza S A, Xu S, Schulz-Heddergott R, Marchenko N D, Moll UM. p53 loss-of-heterozygosity is a necessary prerequisite for mutant p53 stabilization and gain-of-function in vivo. Cell Death Dis. 2017; 8(3):e2661.
36. Gogineni V, Morand S, Staats H, Royfman R, Devanaboyina M, Einloth K, et al. Current Ovarian Cancer Maintenance Strategies and Promising New Developments. J Cancer. 2021; 12(1):38-53.
37. Gorski J W, Ueland F R, Kolesar J M. CCNE1 Amplification as a Predictive Biomarker of Chemotherapy Resistance in Epithelial Ovarian Cancer. Diagnostics (Basel). 2020: 10 (5).
38. Etemadmoghadam D, Weir B A, Au-Yeung G, Alsop K, Mitchell G, George J, et al. Synthetic lethality between CCNE1 amplification and loss of BRCA1. Proc Natl Acad Sci USA. 2013; 110(48):19489-94.
39. Coleman R L, Oza A M, Lorusso D, Aghajanian C, Oaknin A, Dean A, et al. Rucaparib maintenance treatment for recurrent ovarian carcinoma after response to platinum therapy (ARIEL3): a randomised, double-blind, placebo-controlled, phase 3 trial. Lancet. 2017; 390(10106):1949-61.
40. Elyanow R, Wu H T, Raphael B J. Identifying structural variants using linked-read sequencing data. Bioinformatics. 2018; 34(2):353-60.
41. Fang L, Kao C, Gonzalez M V, Mafra F A, Pellegrino da Silva R, Li M, et al. LinkedSV for detection of mosaic structural variants from linked-read exome and genome sequencing data. Nat Commun. 2019; 10(1):5585.
42. Serin Harmanci A, Harmanci A O, Zhou X. CaSpER identifies and visualizes CNV events by integrative analysis of single-cell or bulk RNA-sequencing data. Nat Commun. 2020; 11(1):89.
43. Tirosh I, Izar B, Prakadan S M, Wadsworth M H, 2nd, Treacy D, Trombetta J J, et al. Dissecting the multicellular ecosystem of metastatic melanoma by single-cell RNA-seq. Science. 2016; 352(6282): 189-96.
44. Pignata S, S C C, Du Bois A, Harter P, Heitz F. Treatment of recurrent ovarian cancer. Ann Oncol. 2017; 28(suppl_8): viii51-viii6.
45. Okamoto A, Funakoshi Y, Oe M, Takai R, Suto H, Nagatani Y, et al. Identification of Breast Cancer Stem Cells Using a Newly Developed Long-acting Fluorescence Probe, C5S-A, Targeting ALDHIA1. Anticancer Res. 2022; 42(3):1199-205.
46. Krueger F. Trim Galore, GitHub repository 2016 [Available from: https://github.com/FelixKrueger/TrimGalore.
47. Vasimuddin M, Misra S, Li H, Aluru S. Efficient Architecture-Aware Acceleration of BWA-MEM for Multicore Systems. International Parallel and Distributed Processing Symposium: Rio de Janeiro, Brazil: IEEE: 2019.
48. Broad Institute. Picard Toolkit, GitHub Repository 2019 [Available from: https://broadinstitute.github.io/picard.
49. International HapMap C. The International HapMap Project. Nature. 2003; 426(6968):789-96.
50. Li H, Handsaker B, Wysoker A, Fennell T, Ruan J, Homer N, et al. The Sequence Alignment/Map format and SAMtools. Bioinformatics. 2009; 25(16):2078-9.
51. Langmead B, Salzberg S L. Fast gapped-read alignment with Bowtie 2. Nat Methods. 2012; 9(4):357-9.
52. Gruning B, Dale R, Sjodin A, Chapman B A, Rowe J, Tomkins-Tinch C H, et al. Bioconda: sustainable and comprehensive software distribution for the life sciences. Nat Methods. 2018; 15(7):475-6.
53. Bray N L, Pimentel H, Melsted P, Pachter L. Near-optimal probabilistic RNA-seq quantification. Nat Biotechnol. 2016; 34(5): 525-7.
54. Howe K L, Achuthan P, Allen J, Allen J, Alvarez-Jarreta J, Amode M R, et al. Ensembl 2021. Nucleic Acids Res. 2021; 49(D1):D884-D91.

### EXAMPLE 4—PICOCNV: ALLELE-SPECIFIC SOMATIC COPY NUMBER FROM PICOGRAMS OF DNA

### Summary

Genomic insights from tumour samples comprising just hundreds or even tens of cells hold great clinical potential, but also present significant technical challenges. We previously developed a platform that can accurately identify somatic mutations from such samples. Here, we complete this genomic characterisation with copy number. We present a novel protocol, PicoCNV, to call allele-specific somatic copy number alterations from picogram quantities of tumour DNA. We validate PicoCNV, and demonstrate its clinical potential in maintenance therapy. PicoCNV complements our existing platform, allowing for accurate and comprehensive genomic characterisations of cancers in settings where only microscopic samples are available.

### Introduction

A principal driving force behind each cancer is a repertoire of genomic changes known as somatic alterations^{1,2}. Understanding how these alterations drive cancer is one of the central aims of cancer genomics³, and efforts in this field have brought about clinical benefits including improved patient stratification, new prognostic biomarkers and an arsenal of new therapies^{4,5}. Most somatic alterations of clinical significance are either small somatic mutations (SSMs) or copy number alterations.

Copy number alterations (CNAs) generally refer to large segments of the genome being either duplicated or deleted. They have been found to drive the cancer phenotype^{6,7}, play a prominent role in cancer evolution⁸, and hold substantial prognostic value^{9,10}. They are particularly important in genomically unstable cancer types such as ovarian¹¹, oesophageal¹² and gastric cancers¹³. Due to this importance, researchers have developed many algorithms to call somatic CNAs from sequencing and array data, including ASCAT¹⁴, ABSOLUTE¹⁵, Control-FREEC¹⁶, OncoSNP-SEQ¹⁷, and TITAN¹⁸, among others. In addition to determining the total number of alleles at each locus in the genome, many of these algorithms also determine the numbers of each the two parental alleles. By convention, these are referred to as the major and minor copy numbers. Such methods are said to call allele-specific CNAs. This added allele-specific information allows researchers to identify more subtle CNAs such as copy number neutral loss of heterozygosity (LOH)¹⁴, which can have important clinical implications in cancer¹⁹.

While the analysis of samples derived from bulk tumours is now routine, recent research focus has turned towards microscopic samples, including single cells. For example, studies have investigated the therapeutic opportunities associated with circulating tumour cells²⁰, minimal residual disease^{21,22} and cancer sub-populations such as tumour initiating cells²³. For single cell sequencing, several algorithms have been developed that combine shallow whole genome sequencing (WGS) data across cells to call CNAs in sub-clonal populations. Established algorithms for calling total copy number include Ginkgo²⁴ and AneuFinder²⁵. More recently, researchers have developed allele-specific single cell algorithms, including CHISEL²⁶ and Alleloscope²⁷. However, while single cell methods might provide accurate CNA calls, they are limited in their ability to reliably identify small somatic SSMs²⁸. SSMs are complementary to CNAs and can be highly consequential for therapeutics²⁹. An ideal platform for sequencing microscopic tumour samples would provide both accurate SSMs and CNAs for a complete genomic characterisation. Moreover, many single cell CNA-calling methods rely on aggregating data across thousands of cells, and are therefore unsuitable in settings where total sample size is limited to tens of cells.

To get genomic insights from such microscopic tumour samples, we recently developed a specialised sequencing platform, DigiPico³⁰. We have previously described how DigiPico can be used to investigate active sub-clonal mutational processes in cancer³⁰. Moreover, we estimate that DigiPico has a 76% sensitivity and 95% specificity to detect SSMs (Table S1). Given the biological and prognostic importance of somatic CNAs, we sought to leverage DigiPico's unique features to obtain allele-specific somatic CNAs, thus giving a complete genomic characterisation.

Table S1: Study cohort and per-patient results. Small somatic mutation (SSM) accuracy was calculated using an approach adapted from MutLX, described in³⁰. Signal-to-noise (SNR) calculations are described in FIG. 2C. Copy number alteration (CNA) accuracy was calculated as the percentage of the genome where PicoCNV and bulk ASCAT agreed exactly on both major and minor copy number states. No matched bulk sample was available for patient 11617, so accuracies could not be computed.

| | | | | | | De-noising SNR | | |
|---|---|---|---|---|---|---|---|---|
| | | DigiPico DNA | DigiPico | SSM accuracy (%) | | improvement ratio | | CNA accuracy |
| Patient ID | Tumour histology | input (pg) | wells (n) | Sensitivity | Specificity | RDR | BAF | (% genome) |
| 11152 | High grade serous ovarian carcinoma | 240 | 384 | 54 | 93.6 | 7.02 | 3.68 | 79.3 |
| 11611 | Ovarian carcinosarcoma | 560 | 1152 | 76.7 | 97.8 | 2.24 | 3.89 | 76 |
| 11615 | High grade serous ovarian carcinoma | 900 | 1152 | 93.2 | 94.3 | 4.61 | 3.68 | 87.7 |
| 11619 | High grade fallopian adenocarcinoma | 400 | 1152 | 80.9 | 94.4 | 5.54 | 4.62 | 97.7 |
| 11617 | High grade serous fallopian tube | 1620 | 1152 | — | — | — | — | — |
| | carcinoma | | | | | | | |

### Results

### PicoCNV

Our pipeline to call allele-specific CNAs from DigiPico data consists of two steps: (1) data de-noising and (2) CNA calling (FIG. 15). This two-stage approach was motivated by the observation that raw data from microscopic samples were unsuitable for copy number calling. To obtain CNAs from sequencing data, most algorithms use two quantities calculated along the genome: the read depth ratio (RDR, often transformed to the logR); and the B-allele frequency (BAF). When we calculated these quantities for microscopic samples from Illumina short reads, as is standard for bulk WGS data, they exhibited prohibitively high levels of noise (FIG. 16A). This confirmed the need for a preliminary data de-noising step in our pipeline.

### DigiPico and Matched Bulk Sequencing

We applied DigiPico sequencing to microscopic tumour samples from four patients with ovarian cancer, for whom matched bulk tumour samples were also available (Table S1). The microscopic samples consisted of tumour-initiating cell populations selected by flow cytometry (Methods). Throughout this work, we used CNA calls from ASCAT 14 run on the bulk tumour WGS data as a ground truth against which to measure performance (Methods).

De-Noising with Phase-Based Molecule Counting

DigiPico is a linked-read technology, in which large fragments of DNA (˜100 kb) are distributed into wells, before being amplified, fragmented, barcoded and pooled for WGS³⁰ (FIG. 15). In practice, the Illumina short-read sequencing depth varies greatly between individual large DNA fragments due to non-uniform rates of amplification. This introduces a large amount of noise to the measurement of total sequencing depth along the genome. Since the RDR is calculated as the ratio of tumour and germline sequencing depths, this noise is reflected in the RDR. To counter this effect, we reconstructed the original large DNA fragments in silico (Methods) and used these to calculate the sequencing depth. The number of wells in DigiPico is sufficient that large DNA fragments from overlapping loci are only rarely put into the same well (estimated <5%). Therefore, each locus in the genome is generally covered by at most one large DNA fragment per well. We call this the ‘mono-allelic’ property of DigiPico. Based on this, we reasoned that co-local Illumina short reads in the same well probably originated from the same large DNA fragment (FIG. 15). We then used the resulting in silico reconstructed large fragments (RLFs) to calculate the sequencing depth in the DigiPico samples. This resulted in a visually cleaner RDR track along the genome (FIG. 16B), as well as a five-fold increase in the median signal-to-noise ratio (FIG. 16C, Table S1).

The mono-allelic property of DigiPico also enabled us de-noise the BAF data. Phasing heterozygous SNPs into haplotypes separates the maternal and paternal alleles, allowing for aggregated calculation of allele frequencies^{26,31}. In DigiPico, SNPs that are near to each other and supported by overlapping sets of wells are likely to be on the same allele (FIG. 15). We used this observation to develop a phasing algorithm for DigiPico data (Methods). As with the RDR data, random variations in Illumina short read sequencing depth were a substantial source of noise. We counteracted this noise by deriving BAF values from counts of wells rather than counts of short reads, in addition to haplotype aggregation (Methods). Ultimately, we obtained significantly cleaner BAF tracks along the genome (FIG. 16B). Moreover, we obtained a more than three-fold increase in the median signal-to-noise ratio (FIG. 16C, Table S1). We were therefore satisfied that PicoCNV successfully leveraged the mono-allelic property of DigiPico to de-noise the RDR and BAF data.

### Purity and Ploidy Estimation

We then developed the CNA-calling step of PicoCNV. We followed a well-established approach, first segmenting the RDR and BAF data, before estimating the sample purity and ploidy, and finally fitting copy number states to each segment (Methods).

To produce accurate CNA calls, it is first crucial for any algorithm to correctly estimate the genome-wide average copy number (ploidy) and sample purity. A common difficulty is that high-ploidy solutions often fit data more closely than lower-ploidy ones, even if they do not reflect the true karyotype. To address this, we implemented a heuristic mean squared-error minimisation approach for purity and ploidy estimation, which selected approximately diploid solutions in all samples except one (FIG. 17, Methods). The remaining sample, from patient 11611, was determined to be approximately tetraploid. We validated these ploidy estimates by comparing them to results from ASCAT run on bulk data. The ploidy values agreed for all samples except patient 11619 (FIG. 18A). In this patient, bulk ASCAT gave a tetraploid solution while PicoCNV found a diploid one. However, upon closer inspection, we determined that there was insufficient evidence for tetraploidy in this sample. In particular, ASCAT's tetraploid solution put 99.3% of the genome in a copy number state in which both the major and minor copy numbers were divisible by two. We therefore re-ran ASCAT on this sample with the best diploid solution from ASCAT's grid search. The resulting ploidy value very closely matched PicoCNV's, further indicating that our approach accurately estimated sample ploidy.

### Copy Number Accuracy

Upon manual inspection of the data, we found that a small portion of the genome contained apparently inconsistent RDR and BAF values. We reasoned that this may have reflected sub-clonal CNAs, and we therefore allowed PicoCNV to fit sub-clonal copy number states where fully clonal solutions were a poor fit (Methods). In practice however, more than 95% of the genome was determined to have fully clonal copy number states. We then assessed the accuracy of PicoCNV's copy number calls by comparing them to our ground truth data. We found that there was exact agreement on the copy number state between PicoCNV and bulk ASCAT in 84% of the genome on average (FIG. 18B, Table S1). Moreover, copy number estimates differed by no more than one in roughly 98% of the genome, indicating that the PicoCNV's calls were broadly accurate. To understand how robust these levels of accuracy were, we performed a sub-sampling experiment. In DigiPico, we distribute DNA fragments into one or more 384-well plates. For three of our patients (11611, 11615 and 11619), we had used three plates as we found this improved our ability to detect SSMs (Table S1). However, we found that using one, two or three plates made little difference to PicoCNV's accuracy (FIG. 19). We therefore concluded that PicoCNV was highly robust in its ability to identify CNAs.

We further investigated PicoCNV's ability to detect three categories of CNAs of general interest: amplifications, deletions, and loss of heterozygosity (LOH, Methods). We found that PicoCNV detected the majority of the genome that was deleted (89%) or that had undergone LOH (98%) (FIG. 18C). However, sensitivity for amplifications was lower at 41% This partly reflected the fact that many amplifications were short, with the majority (69%) being less than 1 Mb in length. Since PicoCNV's de-noising step aggregated RDR data into 1 Mb windows, it was unreasonable to expect that we could identify these segments. Indeed, PicoCNV's sensitivity to detect amplifications was strongly dependent on the length of the amplification, rising to 53% for amplifications >10 Mb in length (FIG. 18D). While this did indicate a potential limitation of our approach, overall we were confident that PicoCNV was able to accurately identify the majority of somatic CNAs.

### Application to MRD

Having validated the PicoCNV pipeline, we applied it to a clinically relevant use-case where only microscopic tumour samples were available. Minimal residual disease (MRD) in solid tumours refers to the deposits of cancer cells remaining after a patient has responded well to first-line treatments. MRD is a major source of disease recurrence, so being able to target these cells specifically has the potential to improve patients' clinical outcomes. Indeed, so-called maintenance therapy has recently been adopted for some ovarian cancer patients³². We therefore sought to identify copy number biomarkers that might inform how a patient's maintenance therapy would be managed.

We applied DigiPico sequencing and PicoCNV to an MRD sample taken from patient 11617 (Table S1). PicoCNV produced visually clean RDR and BAF tracks along the genome (FIG. 20A), indicating that data de-noising was effective. Amplifications of CCNE1 are known to indicate a poor prognosis in ovarian cancer³³. Various therapies have been proposed specifically for CCNE1-amplified tumours, including WEE1 kinase and CDK2 inhibitors³³, and proteasome inhibitors³⁴. However, in our MRD sample we found that CCNE1 was not amplified, suggesting that such approaches would not be appropriate (FIG. 20B). A recent phase 3 clinical trial (ARIEL3) found that HRD is predicative of ovarian cancer response to PARP inhibitors for maintenance therapy³⁵. The authors measured HRD using a combination of BRCA mutation status and the extent of genomic LOH. We found that patient 11617 was wild type for both BRCA1 and BRCA2. We then used copy number calls from PicoCNV to measure the extent of LOH across the genome, and found that it was 31.1% (FIG. 20A). This would qualify as LOH-high in the ARIEL3 trial (threshold of 14%), suggesting that patient 11617 might respond well to PARP inhibitors for maintenance therapy. We concluded that PicoCNV was able to provide clinically useful insights in settings where only microscopic samples were available.

### Discussion

We have presented PicoCNV, a new copy number profiling protocol for microscopic cancer samples. PicoCNV uses the mono-allelic property of the DigiPico sequencing platform to de-noise the RDR and BAF data from microscopic tumour samples, before using this data to call CNAs. To our knowledge, this represents the first allele-specific CNA calling protocol that leverages linked-read technology, since previous methods have only provided total copy number 36. We validated PicoCNV's performance by showing that it had high agreement with ground truth results obtained from matched bulk tumour samples. Finally, we demonstrated the clinical utility of PicoCNV by showing how it could be used to inform maintenance therapy in the MRD setting.

This study faced two main limitations, in the nature of the patient cohort and the availability of ground truth data. The cohort of patients with available DigiPico sequencing data was small, consisting of only five patients. Combined with the fact that all samples were taken from ovarian cancers, this ran the risk of developing PicoCNV in a way that would generalise poorly to other data sets. However, our approach used assumptions that depended only on the sequencing platform itself. Moreover, ovarian cancer has one of the highest rates of genomic instability of any cancer type¹¹, and therefore may well have provided a particularly robust setting for developing a CNA calling pipeline. Nevertheless, more work with larger cohort sizes would further validate PicoCNV. Another challenge in this study was the lack of bona fide ground truth data. Using matched bulk data for this purpose was a reasonable choice, but it introduced two potential sources of error. First, while bulk WGS data is generally very reliable, calling CNAs from it is still challenging in some cases. Second, the cells used for DigiPico sequencing represented sub-populations taken from the matched bulk tumours. While it was reasonable to expect that copy number states were the same in the sub-populations and bulk cells, it was possible that certain sub-clonal CNAs were disproportionately selected for or removed from the DigiPico input. Thus, we could not rule out the possibility of biological differences between the matched DigiPico and bulk samples. The accuracy of PicoCNV that we measured therefore likely represents a lower bound on its true performance.

Calling CNAs accurately from microscopic tumour samples is challenging. Single cell methods can provide an alternative approach, depending on researchers' requirements. For example, they provide unparalleled insights into intra-tumour heterogeneity. We note two main differences in the applicability of DigiPico and single cell sequencing approaches. First, single cell methods often need to aggregate data across thousands of individual cells. This is particularly true for shallow sequencing (<0.1× per cell), in which individual cells simply do not have sufficient data to call CNAs at high resolution. On the other hand, we have demonstrated here that DigiPico and PicoCNV can obtain accurate CNA calls starting from as few as 40 cells (Table S1, assuming 6 pg of DNA per cell). Second, a complete genomic characterisation of cancer comprises both copy number states and small somatic mutations (SSMs), i.e. SNVs and indels. Obtaining accurate SSMs from single cell data remains challenging. By contrast, we have developed a machine learning approach, MutLX, to identify somatic SSMs from DigiPico sequencing data³⁰. Internal benchmarking currently indicates that MutLX has a sensitivity of 76% and specificity of 95% for detecting SSMs (Table S1), and this will be the subject of a future manuscript. Combined then, PicoCNV and MutLX will allow for a complete genomic characterisation of cancer starting from microscopic tumour samples. To our knowledge, this is not currently feasible with single cell approaches. Finally, we note that the accuracy of single cell CNA calling methods has generally not been assessed with direct comparison to bulk data, likely due to their emphasis on intra-tumour heterogeneity. Instead, they have been assessed using heuristics^{25,26}, simulation²⁴ and sub-sampling²⁷.

We have demonstrated that PicoCNV is able to accurately and robustly call allele-specific somatic CNAs from microscopic tumour samples. Future studies could use PicoCNV to study minimal residual disease (MRD), circulating tumour cells and other selected tumour cell sub-populations of interest. They could also explore potential applications in liquid biopsies. We could additionally use our platform to examine how acquired resistance to therapies evolves at the MRD stage. In ovarian cancer in particular, where recurrence rates after standard of care are very high (70-80%) 37, this remains a pressing question.

### Code Availability

PicoCNV code is available from https://process.innovation.ox.ac.uk/software, under an academic-use license.

### Methods

### Patient Samples

Written consent from study participants was obtained for participation in the prospective biomarker validation study Gynaecological Oncology Targeted Therapy Study 01 (GO-Target-01) under research ethics approval number 11/SC/0014. Tumour and blood samples were obtained on the day of surgery. Blood samples were processed to isolate peripheral blood mononuclear cells (PBMCs) using Lymphoprep™ (STEMCELL Technologies, Canada). Tumour tissue was dissociated using human Tumor Dissociation Kits (Miltenyi Biotec, Germany) following the manufacturer's protocol. Dissociated tumour cells were processed for staining and fluorescent activated cell sorting to obtain bulk cancer cells and tumour initiating cells (TICs) as described in Okamoto et al.³⁸.

### DNA Extraction and WGS Library Preparation

DNA from PBMCs and bulk cancer cells was isolated using DNeasy blood and tissue kit (Qiagen, USA). Illumina whole-genome sequencing libraries for both germline and tumour DNA were constructed and sequenced by Novogene Co. Ltd (China).

### DigiPico Library Prep and Sequencing

For DigiPico sequencing, DNA was isolated from TICs using Repli-g single cell kits (Qiagen, USA). DigiPico library prep was performed as described in Carrami et al. 30. The libraries were then sent to a sequencing company (Novogene Co. Ltd, China) and were sequenced on a Novaseq platform in 150 bp paired-end sequencing.

### Sequencing Data Pre-Processing

Bulk tumour and normal blood-derived whole genome sequencing data were trimmed of Illumina adapters with Trim Galore v0.6.6³⁹ and aligned to the human genome version hg19 using Bwa-mem v2.2.1⁴⁰. PCR duplicate reads were removed from the resulting BAM files using Picard Tools v2.18.17⁴¹.

For DigiPico data, raw paired-end whole genome sequencing data were demultiplexed to give two FASTQ files per well using custom scripts. Reads from each well were then trimmed of Illumina adapters using Trim Galore v0.6.6³⁹ and aligned to the human genome version hg19 using Bowtie v2.4.1⁴². The resulting BAM files were cleaned of PCR duplicates with Picard Tools v2.18.17⁴¹. The per-well BAM files were merged with samtools, using read groups to keep track of the wells that each read originated from.

### Bulk ASCAT Implementation

A set of common SNPs was obtained from HapMap v3.3⁴³. These were genotyped in bulk tumour and matched germline WGS data using Platypus⁴⁴ to produce allele-specific read counts in both samples. Retained heterozygous SNPs consisted of those with at least 20 covering reads and an allele frequency of between 25% and 75% in the germline sample. Tumour and germline BAFs were calculated for these SNPs from the read counts. LogR was calculated at each SNP as

$${log}_{2} ( \frac{tumour reads}{germline reads} ) ,$$

and centred to have zero median for each patient.

ASCAT¹⁴ v2.5.2 was obtained from Bioconda⁴⁵. The ‘Standard ASCAT run’ pipeline from https://github.com/VanLoo-lab/ascat/tree/master/ExampleData was used to call CNAs, including GC-wave correction and with the compaction parameter gamma set to 1.

### PicoCNV RDR Calculation

The original large DNA fragments from the DigiPico protocol were reconstructed in silico to give reconstructed large fragments (RLFs). Separately for each chromosome in each well, read pairs were determined to come from the same RLF if the gap between them was less than 100 kb. Each RLF spanned the from start of the first read belonging to it, to the end of the last read.

The PicoCNV read depth ratio (RDR) was calculated in non-overlapping 1 Mb windows along the genome for DigiPico samples. Within each window, the raw RDR was calculated as

$$r = 100 \times \frac{average RLF depth}{germline reads}$$

It was then corrected for GC content and mappability using a multivariate linear model trained separately for each sample.

### PicoCNV SNP Phasing and BAF Calculation

A set of common SNPs was obtained from dbSNP v138⁴⁶, and retained as heterozygous for each patient if in the germline data they had a sequencing depth of at least 20 reads and an allele frequency of between 25% and 75%. Phasing each patient's heterozygous SNPs from DigiPico tumour sequencing data consisted of two steps: partitioning the genome into blocks; and assigning SNPs within each block to one of two haplotypes.

Potential blocks of SNPs were first identified as contiguous regions where the gap from one SNP to the next was no greater than 500 kb. Blocks containing more than 100 SNPs were subdivided so that they did not exceed this limit. Blocks were then checked for connectivity. A block was disconnected if there existed a sub-partitioning such that the SNPs in each partition did not share any covering wells with the other partitions. Disconnected blocks were sub-divided into these partitions. Finally, any remaining singleton blocks consisting of only one SNP were removed.

Within each block, SNPs were then assigned to one of two haplotypes. To do this, the SNP-SNP similarity matrix M was first constructed as

$${M}_{i j} = \frac{{\sum }_{k} {G}_{i k} {G}_{j k}}{{\sum }_{l} \vert {G}_{i l} \vert \vert {G}_{j l} \vert} , where {G}_{i k} = \{ \begin{matrix}+ 1 , if SNP i is alt in well k \\ - 1 , if SNP i is ref in well k \\ 0 , if SNP i is not covered by well k\end{matrix}$$

M contains values between +1 and −1, with Mᵢⱼ=+1 if SNPs i and j have the same status in all wells, and Mᵢⱼ=−1 if they are different in all wells. It can be shown that, if the mono-allelic property of DigiPico holds exactly, then Mᵢⱼ=hᵢhⱼ, where h is a haplotype vector indicating which haplotype each SNP belongs to with entries ±1. By convention, we always take the first entry in h to be positive. This matrix outer product can be efficiently computed by applying singular value decomposition to M. In particular, the first singular vector h* of M is a close approximation to h, and the final estimate of h is

$${h}_{i} = sgn ( {h}_{i}^{*} ) .$$

The PicoCNV BAF value was then calculated for each haplotype group as

$$b = \frac{{\sum }_{S N P s} wells supporting alt allele}{{\sum }_{S N P s} wells covering SNP}$$

using the median genomic coordinate of all SNPs in the group as the position of the group.

### Segmentation

The genome was segmented first using the BAF data alone, and then further segmented using the RDR data alone. This reflected the observation that the BAF data tended to be cleaner than the RDR data, even after data de-noising. Prior to segmentation, BAF data b were mirrored according to bₘᵢᵣᵣ=0.5−|0.5−b|, and RDR data r were normalised according to rₙₒᵣₘ=r/4r̅. Here, r̅ is the genome-wide average of the corrected RDR. This normalisation ensured that the RDR and mirrored BAF data were on comparable scales.

Segmentation was performed in each chromosome arm separately. Segmentation of data x in each arm was performed by minimising the loss function

$$L ( x ) = {\sum }_{s ∈ S} {\sum }_{i ∈ s} {( {w}_{i} {x}_{i} - {\overline{x}}_{s} )}^{2} + λ \vert S \vert$$

Here, s indexes segments, S is the set of all segments on the chromosome arm, wᵢ is a normalised weight per data point (size of haplotype group for BAF data, 1 for RDR data), x̅ₛ is the per-segment weighted mean, and λ is a parameter that we set to 0.1. Segment lengths were kept above a minimum of 5 Mb. Minimisation was carried out by a stochastic greedy search algorithm. To ensure that we found a global minimum of the loss function, we implemented the greedy search 10,000 times and took the best resulting segmentation.

### Purity and Ploidy Grid Search

For a range of values of sample purity φ and tumour ploidy ψ, the genome-wide mean squared error (MSE) was calculated. This MSE was calculated as a length-normalised sum over segments,

$$MSE = \frac{{\sum }_{s} length ( s ) {MSE}_{s}}{{\sum }_{s} length ( s )}$$

Within each segment s, the MSE was a sum of terms from the RDR and mirrored BAF,

$$M S {E}_{s} = M S {E}_{s}^{( r )} + M S {E}_{s}^{( b )}$$

$$with$$

$$MS {E}_{s}^{( x )} = \frac{1}{\vert s \vert} {\sum }_{i ∈ s} \frac{{( {x}_{i} - {\hat{x}}_{s} )}^{2}}{{\hat{σ}}_{x}^{2}}$$

Here, |s| is the number of data points in s,

$${\hat{σ}}_{x}^{2}$$

is a genome-wide estimate of the variance of x using deviation from per-segment means, and the estimators \hat{x}ₛ for RDR and BAF are functions of the copy number state. If a state consists of major and minor copy numbers n_{A} and n_{B} respectively, then

$${\hat{r}}_{s} = \overline{r} \frac{ϕ ( {n}_{A} + {n}_{B} ) + 2 ( 1 - ϕ )}{ϕ ψ + 2 ( 1 - ϕ )} and {\hat{b}}_{s} = \frac{ϕ {n}_{B} + ( 1 - ϕ )}{ϕ ( {n}_{A} + {n}_{B} ) + 2 ( 1 - ϕ )}$$

assuming that non-cancer cells are diploid. A finite set of copy number states were considered for each segment. This set consisted of all possible states with total copy number up to the largest value consistent with the RDR observed for the entire genome. In each segment, the state with the smallest MSE was selected, and the resulting whole-genome MSE was then calculated as detailed above.

Selecting the (φ, ψ) pair with the minimal MSE would often result in erroneously tetraploid solutions. To counter this, we identified local MSE minima from the grid search. If it was sufficiently pronounced, the lowest-ploidy MSE minimum was taken as the solution (FIG. 17B).

### Sub-Clonal Copy Number Fitting

Given values for φ and ψ from the grid search, final copy number states were fit using a modified version of the MSE-minimisation approach above. In particular, the set of possible copy number states for each segment was expanded to include clonalities χ∈{0.5, 0.6, 0.7, 0.8, 0.9, 1}. We modelled tumour cells without a particular CNA as having the modal copy number state {m_{A}, m_{B}} from the fully clonal solution. The RDR and BAF estimators were therefore changed to

$${\hat{r}}_{s} = \overline{r} \frac{ϕ χ ( {n}_{A} + {n}_{B} ) + ϕ ( 1 - χ ) ( {m}_{A} + {m}_{B} ) + 2 ( 1 - ϕ )}{ϕ ψ + 2 ( 1 - ϕ )}$$

$${\hat{b}}_{s} = \frac{ϕ χ {n}_{B} + ϕ ( 1 - χ ) {m}_{B} + ( 1 - ϕ )}{ϕ χ ( {n}_{A} + {n}_{B} ) + ϕ ( 1 - χ ) ( {m}_{A} + {m}_{B} ) + 2 ( 1 - ϕ )}$$

To avoid over-fitting of sub-clonal solutions, a penalty term was added to the per-segment MSE so that

$$M S {E}_{s} = M S {E}_{s}^{( r )} + M S {E}_{s}^{( b )} + π ( χ ) , where π ( χ ) = \{ \begin{matrix}0 , & if χ = 1 \\ 10 , & otherwise\end{matrix}$$

The copy number state with the smallest value of MSEₛ was chosen for each segment.

### Sensitivity for Amplifications, Deletions and LOH

For each patient, the median total copy number n_{med} was calculated for both PicoCNV and bulk ASCAT calls. Amplifications were then identified as contiguous regions with total copy number ≥2n_{med}. Deletions were regions with total copy number 0, and LOH was defined as n_{B}=0 with n_{A}>0. We measured PicoCNV's sensitivity to detect these events as

$$\frac{\# bases where PicoCNV and bulk ASCAT agreed on event}{\# bases where bulk ASCAT called the event}$$

### REFERENCES

1 Bailey, M. H. et al. Comprehensive Characterization of Cancer Driver Genes and Mutations. Cell 173, 371-385 e318, doi: 10.1016/j.cell.2018.02.060 (2018).
2 Consortium, I. T. P.-C. A. o. W. G. Pan-cancer analysis of whole genomes. Nature 578, 82-93, doi: 10.1038/s41586-020-1969-6 (2020).
3 Vogelstein, B. et al. Cancer genome landscapes. Science 339, 1546-1558, doi: 10.1126/science. 1235122 (2013).
4 Berger, M. F. & Mardis, E. R. The emerging clinical relevance of genomics in cancer medicine. Nat Rev Clin Oncol 15, 353-365, doi: 10.1038/s41571-018-0002-6 (2018).
5 Malone, E. R., Oliva, M., Sabatini, P. J. B., Stockley, T. L. & Siu, L. L. Molecular profiling for precision cancer therapies. Genome Med 12, 8, doi: 10.1186/s13073-019-0703-1 (2020).
6 Sondka, Z. et al. The COSMIC Cancer Gene Census: describing genetic dysfunction across all human cancers. Nat Rev Cancer 18, 696-705, doi: 10.1038/s41568-018-0060-1 (2018).
7 Wang, G. & Anastassiou, D. Pan-cancer driver copy number alterations identified by joint expression/CNA data analysis. Sci Rep 10, 17199, doi: 10.1038/s41598-020-74276-6 (2020).
8 Gerstung, M. et al. The evolutionary history of 2,658 cancers. Nature 578, 122-128, doi: 10.1038/s41586-019-1907-7 (2020).
9 Hieronymus, H. et al. Tumor copy number alteration burden is a pan-cancer prognostic factor associated with recurrence and death. Elife 7, doi: 10.7554/eLife.37294 (2018).
10 Smith, J. C. & Sheltzer, J. M. Systematic identification of mutations and copy number alterations associated with cancer patient prognosis. Elife 7, doi: 10.7554/eLife.39217 (2018).
11 Penner-Goeke, S. et al. The temporal dynamics of chromosome instability in ovarian cancer cell lines and primary patient samples. PLoS Genet 13, e1006707, doi: 10.1371/journal.pgen. 1006707 (2017).
12 Paulson, T. G. et al. Chromosomal instability and copy number alterations in Barrett's esophagus and esophageal adenocarcinoma. Clin Cancer Res 15, 3305-3314, doi: 10.1158/1078-0432.CCR-08-2494 (2009).
13 Maleki, S. S. & Rocken, C. Chromosomal Instability in Gastric Cancer Biology. Neoplasia 19, 412-420, doi: 10.1016/j.neo.2017.02.012 (2017).
14 Van Loo, P. et al. Allele-specific copy number analysis of tumors. Proc Natl Acad Sci USA 107, 16910-16915, doi: 10.1073/pnas. 1009843107 (2010).
15 Carter, S. L. et al. Absolute quantification of somatic DNA alterations in human cancer. Nat Biotechnol 30, 413-421, doi: 10.1038/nbt.2203 (2012).
16 Boeva, V. et al. Control-FREEC: a tool for assessing copy number and allelic content using next-generation sequencing data. Bioinformatics 28, 423-425, doi: 10.1093/bioinformatics/btr670 (2012).
17 Yau, C. OncoSNP-SEQ: a statistical approach for the identification of somatic copy number alterations from next-generation sequencing of cancer genomes. Bioinformatics 29, 2482-2484, doi: 10.1093/bioinformatics/btt416 (2013).
18 Ha, G. et al. TITAN: inference of copy number architectures in clonal cell populations from tumor whole-genome sequence data. Genome Res 24, 1881-1893, doi: 10.1101/gr.180281.114 (2014).
19 Ryland, G. L. et al. Loss of heterozygosity: what is it good for? BMC Med Genomics 8, 45, doi: 10.1186/s12920-015-0123-z (2015).
20 Lin, D. et al. Circulating tumor cells: biology and clinical significance. Signal Transduct Target Ther 6, 404, doi: 10.1038/s41392-021-00817-8 (2021).
21 Artibani, M. et al. Adipocyte-like signature in ovarian cancer minimal residual disease identifies metabolic vulnerabilities of tumor-initiating cells. JCI Insight 6, doi: 10.1172/jci.insight. 147929 (2021).
22 Luskin, M. R., Murakami, M. A., Manalis, S. R. & Weinstock, D. M. Targeting minimal residual disease: a path to cure? Nat Rev Cancer 18, 255-263, doi: 10.1038/nrc.2017.125 (2018).
23 Qureshi-Baig, K., Ullmann, P., Haan, S. & Letellier, E. Tumor-Initiating Cells: a criTICal review of isolation approaches and new challenges in targeting strategies. Mol Cancer 16, 40, doi: 10.1186/s12943-017-0602-2 (2017).
24 Garvin, T. et al. Interactive analysis and assessment of single-cell copy-number variations. Nat Methods 12, 1058-1060, doi: 10.1038/nmeth.3578 (2015).
25 Bakker, B. et al. Single-cell sequencing reveals karyotype heterogeneity in murine and human malignancies. Genome Biol 17, 115, doi: 10.1186/s13059-016-0971-7 (2016).
26 Zaccaria, S. & Raphael, B. J. Characterizing allele- and haplotype-specific copy numbers in single cells with CHISEL. Nat Biotechnol 39, 207-214, doi: 10.1038/s41587-020-0661-6 (2021).
27 Wu, C. Y. et al. Integrative single-cell analysis of allele-specific copy number alterations and chromatin accessibility in cancer. Nat Biotechnol 39, 1259-1269, doi: 10.1038/s41587-021-00911-w (2021).
28 Dong, X. et al. Accurate identification of single-nucleotide variants in whole-genome-amplified single cells. Nat Methods 14, 491-493, doi: 10.1038/nmeth.4227 (2017)
29 Morotti, M. et al. Promises and challenges of adoptive T-cell therapies for solid tumours. Br J Cancer 124, 1759-1776, doi: 10.1038/s41416-021-01353-6 (2021).
30 KaramiNejadRanjbar, M. et al. A highly accurate platform for clone-specific mutation discovery enables the study of active mutational processes. Elife 9, doi: 10.7554/eLife.55207 (2020).
31 Nik-Zainal, S. et al. The life history of 21 breast cancers. Cell 149, 994-1007, doi: 10.1016/j.cell.2012.04.023 (2012).
32 Gogineni, V. et al. Current Ovarian Cancer Maintenance Strategies and Promising New Developments. J Cancer 12, 38-53, doi: 10.7150/jca.49406 (2021).
33 Gorski, J. W., Ueland, F. R. & Kolesar, J. M. CCNE1 Amplification as a Predictive Biomarker of Chemotherapy Resistance in Epithelial Ovarian Cancer. Diagnostics (Basel) 10, doi: 10.3390/diagnostics10050279 (2020).
34 Etemadmoghadam, D. et al. Synthetic lethality between CCNE1 amplification and loss of BRCA1. Proc Natl Acad Sci USA 110, 19489-19494, doi: 10.1073/pnas. 1314302110 (2013).
35 Coleman, R. L. et al. Rucaparib maintenance treatment for recurrent ovarian carcinoma after response to platinum therapy (ARIEL3): a randomised, double-blind, placebo-controlled, phase 3 trial. Lancet 390, 1949-1961, doi: 10.1016/S0140-6736(17)32440-6 (2017).
36 Marks, P. et al. Resolving the full spectrum of human genome variation using Linked-Reads. Genome Res 29, 635-645, doi: 10.1101/gr.234443.118 (2019).
37 Pignata, S., S, C. C., Du Bois, A., Harter, P. & Heitz, F. Treatment of recurrent ovarian cancer. Ann Oncol 28, viii51-viii56, doi: 10.1093/annonc/mdx441 (2017).
38 Okamoto, A. et al. Identification of Breast Cancer Stem Cells Using a Newly Developed Long-acting Fluorescence Probe, C5S-A, Targeting ALDHIA1. Anticancer Res 42, 1199-1205, doi: 10.21873/anticanres. 15586 (2022).
39 Krueger, F Trim Galore. GitHub repository, <https://github.com/FelixKrueger/TrimGalore> (2016).
40 Vasimuddin, M., Misra, S., Li, H. & Aluru, S. in International Parallel and Distributed Processing Symposium (IEEE, Rio de Janeiro, Brazil, 2019).
41 Broad Institute. Picard Toolkit. GitHub Repository, <https://broadinstitute.github.io/picard> (2019).
42 Langmead, B. & Salzberg, S. L. Fast gapped-read alignment with Bowtie 2. Nat Methods 9, 357-359, doi: 10.1038/nmeth. 1923 (2012).
43 International HapMap, C. The International HapMap Project. Nature 426, 789-796, doi: 10.1038/nature02168 (2003).
44 Rimmer, A. et al. Integrating mapping-, assembly- and haplotype-based approaches for calling variants in clinical sequencing applications. Nat Genet 46, 912-918, doi: 10.1038/ng.3036 (2014).
45 Gruning, B. et al. Bioconda: sustainable and comprehensive software distribution for the life sciences. Nat Methods 15, 475-476, doi: 10.1038/s41592-018-0046-7 (2018).
46 Sherry, S. T. et al. dbSNP: the NCBI database of genetic variation. Nucleic Acids Res 29, 308-311, doi: 10.1093/nar/29.1.308 (2001).

All references herein are incorporated by reference.

1. A method of determining somatic allele-specific copy number alterations (CNAs) in the genomes of cells in a test-sample from a subject, the method comprising:
i) providing an indexed-DNA library of DNA fragments resulting from whole-genome amplification of genomic DNA from cells of the test-sample, wherein fragments of DNA in the indexed-DNA library are distributed among, and are indexed to, individual wells, and the fragments are at least 40 kb in length;
ii) providing whole genome sequencing data of reference non-cancer cells from a reference-sample from the subject; and
iii) determining somatic allele-specific copy number alterations in the genome(s) of the cells of the test-sample by:
a) determining a read depth ratio (RDR), which is the ratio of read counts of an allele in the test-sample relative to the reference-sample, or a log ratio (LogR) thereof, thereby providing the total copy number of the allele, wherein the RDR or LogR value is derived from counts of the allele in reconstructed fragments of DNA that have been reconstructed in silico, and wherein the counts are within a bin of between 100 kb and 5 Mb, or between 80 kb and 120 kb, on the genome; and
b) determining a B-allele frequency (BAF) value, which is the allelic ratio of parental alleles determined by the ratio of the wells supporting a reference allele versus a non-reference allele and averaged over phased groups of heterozygous single nucleotide polymorphisms (SNPs); and
c) using the determined RDR or logR, and the BAF value to call allele-specific somatic CNAs via an allele specific CNA calling algorithm.

2. The method according to claim 1, wherein the allele-specific CNA-calling algorithm comprises or consists of a mean-squared error (MSE)-minimisation algorithm or TITAN algorithm.

3. The method according to claim 2, wherein the TITAN algorithm has a tuneable segment length parameter set within a range of 10²⁰ to 10²³.

4. The method according to any preceding claim, wherein two or more sequences of DNA from the same well may be determined to be a read pair from the same in silico reconstructed fragment if there is a gap between them shorter than 10 kb in length.

5. The method according to any preceding claim, wherein providing an indexed-DNA library resulting from whole-genome amplification of genomic DNA from cells in the test-sample is by carrying out, or obtaining results from, a method of whole genome sequencing of the cells comprising the steps of:
i) providing a multi-well array plate comprising rows and columns of reaction wells;
ii) providing genomic DNA of cells of a test-sample, wherein the genomic DNA is distributed into a plurality of reaction wells on the multi-well array plate, such that there is no more than one single-stranded genomic DNA molecule of any given locus per reaction well,
iii) carrying out whole genome amplification (WGA) of each genomic DNA molecule to provide multiple copies of the genomic DNA molecule in each reaction well;
iv) fragmenting the DNA molecules of each reaction well and ligating a pair of looped adapters at each end or tagmenting using transposase-delivered adapters to form adapted-DNA fragments, wherein the looped adapters or transposase-delivered adapters comprise either a Column Index (Ci) sequence or a Row Index (Ri) sequence, wherein the Ci sequence is common to each looped adapter or transposase-delivered adapter of every reaction well in a column of the multi-well array plate, or wherein each Ri sequence is common to each looped adapter or transposase-delivered adapter of every reaction well in a row of the multi-well array plate;
vi) providing the indexed DNA library by performing indexing PCR on the adapted-DNA fragments, wherein the adapted-DNA fragments are amplified to form indexed PCR products using forward and reverse indexing primers, wherein either a Row Index (Ri) sequence or Column Index (Ci) sequence is introduced by each forward and reverse indexing primers onto each end of the adapted-DNA fragments, such that the resulting indexed PCR products comprise both a pair of flanking Column Index (Ci) sequences that are common to each well of a column and a pair of flanking Row Index (Ri) sequences that are common to each well of a row, and
vii) sequencing the indexed DNA library.

6. The method according to any preceding claim, wherein the cells are cancerous cells, or pre-cancerous cells.

7. The method according to any preceding claim, wherein the cells are laser-captured micro-dissected cells or circulating tumour cells.

8. The method according to any preceding claim, wherein the cells from the reference-sample are cells from non-tumour-containing tissue or blood from the subject.

9. The method according to any preceding claim, wherein the method further comprises sequencing the indexed-DNA library of DNA fragments to provide data for determining any single nucleotide polymorphisms (SNPs) in the genome of cells.

10. A method for monitoring cancer in a subject, or monitoring or detecting minimal residual disease (MRD) or cancer relapse in a subject, the method comprising:
determining somatic allele-specific copy number alterations (CNAs) in the genomes of cells in a test-sample from the subject in accordance with any of claims 1-9.

11. The method according to claim 10, wherein the subject has received previous cancer therapy; optionally wherein the subject is in a state of remission.

12. The method according to any of claims 10-12, wherein the method further comprises treatment of the subject, such as administration of cancer therapy, if they are determined to have a progressing cancer, MRD or cancer relapse.

13. A method of cancer therapy for a subject, the method comprising obtaining or receiving results of a method carried out in accordance with any of claims 1-10; wherein if the subject is determined to have a progressing cancer, MRD or cancer relapse, treating the subject, such as by administration of cancer therapy.

14. The method according to claim 12 or 13, wherein the cancer therapy comprises one or more of chemotherapy, radiotherapy, surgery, CAR-T therapy, and anti-cancer vaccination.

15. Use of the method according to any of claims 1-9 for monitoring cancer in a subject, such as monitoring progression of cancer; or monitoring or detecting minimal residual disease (MRD) or cancer relapse in a subject.
